(19) United States
(12) Patent Application Publication
(10) Pub. No.: US 2005/0065139 A1
(43) Pub. Date: Mar. 24, 2005

(54) Glycosidase inhibitors and methods of synthesizing same

(76) Inventors: Brian Mario Pinto, Coquitlam (CA); Blair D. Johnston, Vancouver (CA); Monica Gabriela Szczepina, Vancouver (CA); Hui Liu, Coquitlam (CA); Kashinath Sadalapure, Vancouver (CA); Ahmad Ghavami, Guelph (CA)

Correspondence Address: CHERNOFF, VILHAUER, MCCLUNG & STENZEL, 1600 ODS TOWER, 601 SW SECOND AVENUE, PORTLAND, OR, 97204-3157, US

(21) Appl. No.: 10/877,490

(22) Filed: Jun. 25, 2004

Related U.S. Application Data
(63) Continuation-in-part of application No. 10/226,657, filed on Aug. 22, 2002, now abandoned.
Application No. 10/226,657 is a continuation of application No. 09/627,434, filed on Jul. 28, 2000, now Pat. No. 6,455,573.
(60) Provisional application No. 60/482,006, filed on Jun. 25, 2003.

(51) Int. Cl. A61K 31/4015; A61K 31/381; C07D 345/00

(52) U.S. Cl. 514/183; 514/424; 514/445; 540/1; 548/542; 549/62

(57) ABSTRACT

A method for synthesizing Salacinol, its stereoisomers, and analogues, homologues and other derivatives thereof potentially useful as glycolsidase inhibitors. The compounds of the invention may have the general formula (I) or (II): 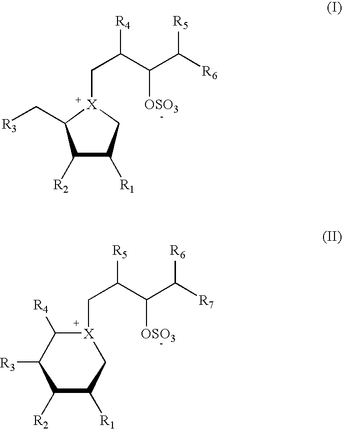 The synthetic schemes comprise reacting a cyclic sulfate with a 5-membered ring sugar containing a heteroatom (X). The heteroatom preferably comprises sulfur, selenium, or nitrogen. The cyclic sulfate and ring sugar reagents may be readily prepared from carbohydrate precursors, such as D-glucose, L-glucose, D-xylose and L-xylose. The target compounds are prepared by opening of the cyclic sulfates by nucleophilic attack of the heteroatoms on the 5-membered ring sugars. The resulting heterocyclic compounds have a stable, inner salt structure comprising a heteroatom cation and a sulfate anion. The synthetic schemes yield various stereoisomers of the target compounds in moderate to good yields with limited side-reactions.

### RELATED APPLICATIONS

This application is a continuation-in-part of U.S. patent application Ser. No. 10/226,657, which is a continuation of U.S. patent application Ser. No. 09/627,434, now issued as U.S. Pat. No. 6,455,573. This application claims priority from U.S. Provisional Patent Application No. 60/482,006 which is hereby incorporated by reference.

### TECHNICAL FIELD

This application relates to methods for synthesizing Salacinol, its stereoisomers, and analogues, homologues and other derivatives thereof potentially useful as glycosidase inhibitors.

### BACKGROUND

In treatment of non-insulin dependent diabetes (NIDD) management of blood glucose levels is critical. One strategy for treating NIDD is to delay digestion of ingested carbohydrates, thereby lowering post-prandial blood glucose concentration. This can be achieved by administering drugs which inhibit the activity of enzymes, such as glucosidases, which mediate the hydrolysis of complex starches to oligosaccharides in the small intestine. For example, carbohydrate analogues, such as Acarbose, reversibly inhibit the function of pancreatic α-amylase and membrane-bound intestinal α-glucoside hydrolase enzymes. In patients suffering from Type II diabetes, such enzyme inhibition results in delayed glucose absorption into the blood and a smoothing or lowering of postprandial hyperglycemia, resulting in improved glycemic control.

Some naturally-occurring glucosidase inhibitors have been isolated from Salacia reticulata, a plant native to submontane forests in Sri Lanka and parts of India (known as “Kotala himbutu” in Singhalese). Salacia reticulata is a woody climbing plant which has been used in the Ayurvedic system of Indian medicine in the treatment of diabetes. Traditionally, Ayurvedic medicine advised that a person suffering from diabetes should drink water left overnight in a mug carved from Kotala himbutu wood. In an article published in 1997, Yoshikawa et al. reported the isolation of the compound Salacinol from a water-soluble fraction derived from the dried roots and stems of Salacia reticulata.¹ Yoshikawa et al. determined the structure of Salacinol, shown below, and demonstrated its efficacy as an α-glucosidase inhibitor.

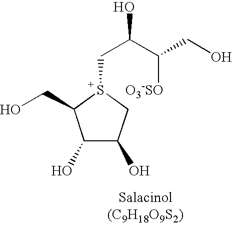

Yoshikawa et al. later reported the isolation from the roots and stems of Salacia reticulata of Kotalanol which was also shown to be effective as an α-glucosidase inhibitor.² Like Salicinol, Kotalanol contains a thiosugar sulfonium ion and an internal sulfate providing the counterion: 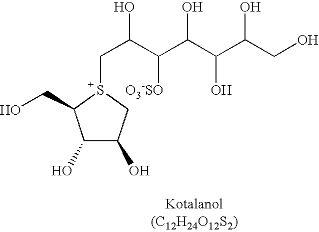
Kotalanol has been found to show more potent inhibitory activity against sucrase than Salicinol and Acarbose.²

The exact mechanism of action of Salacinol and other glucosidase inhibitors has not yet been elucidated. Some known glycosidase inhibitors, such as the indolizidine alkaloids castanospermine and swainsonine, are known to carry a positive charge at physiological pH. 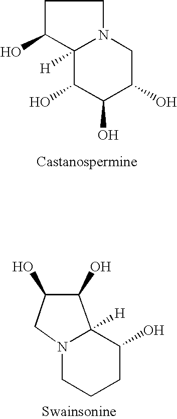
It is believed that the mechanism of action of some known inhibitors may be at least partially explained by the establishment of stabilizing electrostatic interactions between the inhibitor and the enzyme active site carboxylate residues. It is postulated that the compounds of the present invention, which comprise postively charged sulfonium, ammonium, and selenonium ions, could function in a similar manner. It is also possible that Salacinol and other compounds of the same class may act by alteration of a transport mechanism across the intestinal wall rather than by directly binding to glucosidase enzymes.

Salacinol and Kotalanol may potentially have fewer long-term side effects than other existing oral antidiabetic agents. For example, oral administration of Acarbose in the treatment of Type II diabetes results in undesirable gastrointestinal side effects in some patients, most notably increased flatulence, diarrhoea and abdominal pain. As mentioned above, Salacinol has been used as a therapy for diabetes in the Ayurvedic system of traditional medicine for many years with no notable side effects reported. Further, recent animal studies have shown that the oral ingestion of an extractive from a Salacia reticulata trunk at a dose of 5,000 mg/kg had no serious acute toxicity or mutagenicity in rats.³

The Salacia reticulata plant is, however, in relatively small supply and is not readily available outside of Sri Lanka and India. Accordingly, it would be desirable if Salicinol, Kotalanol and analogues thereof could be produced synthetically.

Carbohydrate processing inhibitors have also been shown to be effective in the treatment of some non-diabetic disorders, such as cancer. While normal cells display characteristic oligosaccharide structures, tumor cells display very complex structures that are usually found in embryonic tissues. It is believed that these complex structures provide signal stimuli for rapid proliferation and metastasis of tumor cells. A possible strategy for therapeutic use of glucosidase inhibitors is to take advantage of the differential rates of normal vs cancer cell growth to inhibit assembly of complex oligosaccharide structures. For example, the indolizidine alkaloid swainsonine, an inhibitor of Golgi α-mannosidase II, reportedly reduces tumor cell metastasis, enhances cellular immune responses, and reduces tumor cell growth in mice.⁴ Swainsonine treatment has led to significant reduction of tumor mass in human patients with advanced malignancies, and is a promising drug therapy for patients suffering from breast, liver, lung and other malignancies.^{5,6}

The compounds of the present invention may also find application in the treatment of Alzheimer's disease due to their stable, internal salt structure. Alzheimer's is characterized by plaque formation in the brain caused by aggregation of a peptide, β-amyloid, into fibrils. This is toxic to neuronal cells. One can inhibit this aggregation by using detergent-like molecules. It is believed that the compounds of the present invention, which are amphipathic, may demonstrate this activity.

The need has therefore arisen for a new class of glycosidase inhibitors which may be synthesized in high yields from readily available starting materials and which have potential use as therapeutics.

### SUMMARY OF THE INVENTION

In accordance with the invention, a compound selected from the group consisting of non-naturally occurring compounds represented by the general formula (I), including stereoisomers and pharmaceutically acceptable salts thereof is disclosed, 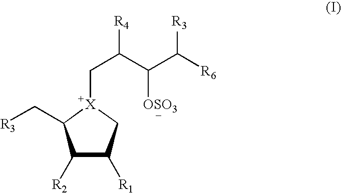
where X is selected from the group consisting of S, Se, and NH. Such compounds include stereoisomers of Salicinol. The target compounds have a stable, internal salt structure comprising heteroatom cation X and a sulfate anion; the substituents may vary without departing from the invention. Preferably, R₁, R₂, R₃, R₄ and R₅ are the same or different and are selected from the group consisting of H, OH, SH, NH₂, halogens and constituents of compounds selected from the group consisting of cyclopropanes, epoxides, aziridines and episulfides; and R₆ is selected from the group consisting of H and optionally substituted straight chain, branched, or cyclic, saturated or unsaturated hydrocarbon radicals, such as alkyl, alkenyl, alkynyl, aryl, and alkoxy substituents containing any suitable functionality. In one embodiment of the invention R₆ may be a polyhydroxylated, acyclic chain, such as an alditol chain of between 5 and 10 carbons.

In another embodiment of the invention, the heterocycle ring may comprise 6 rather 5 carbons and the compound may be represented by the general formula (II):

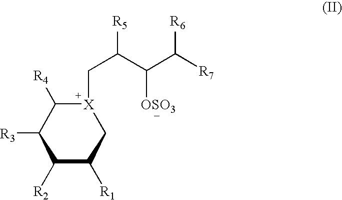

Processes for the production of compounds of the general formula (I) and (II) are also disclosed comprising reacting a cyclic sulfate having the general formula (III) with a 5-membered ring sugar having the general formula (IV) or (V) 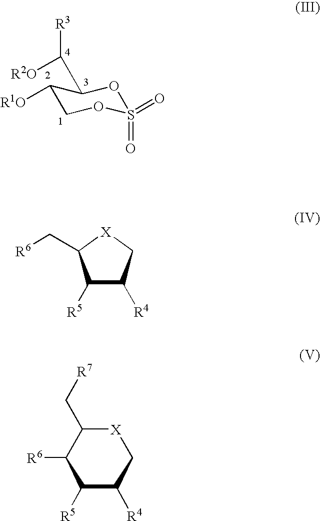
where X is selected from the group consisting of S, Se, and NH; R¹ and R² are selected from the group consisting of H and a protecting group; R³ is selected from the group consisting of H and optionally substituted straight chain, branched, or cyclic, saturated or unsaturated hydrocarbon radicals and their protected derivatives; and R⁴, R⁵ and R⁶ are the same or different and are selected from the group consisting of H, OH, SH, NH₂, halogens and constituents of compounds selected from the group consisting of cyclopropanes, epoxides, aziridines and episulfides and their protected derivatives. Preferably the cyclic sulfate is a 2,4-di-O-protected-D-or L-erythritol-1,3-cyclic sulfate, such as 2,4-O-Benzylidene-D-or L-erythritol-1,3-cyclic sulfate (i.e. R¹ and R² comprise a benzylidene protecting group); R³ is H or a protected polyhydroxylated alkyl chain; and R⁴, R⁵ and R⁶ are selected from the group consisting of OH and a protected OH group, such as OCH₂C₆H₅ or OCH₂C₆H₄OCH₃. The synthetic processes comprise the step of opening the cyclic sulfate (III) by nucleophilic attack of the heteroatom X on the sugar (IV) or (V).

The processes for the production of the target compounds may include the use of novel protecting and deprotecting agents, such as p-methoxybenzyl, and solvents, such as hexafluoroisopropanol.

The application also relates to the use of a compound according to formula (I) or (II) as a glycosidase inhibitor, and to pharmaceutical compositions comprising an effective amount of a compound according to formula (I) or (II), or combinations thereof, together with a pharmaceutically acceptable carrier, and to methods of treating carbohydrate metabolic disorders, such as non-insulin dependent diabetes by administering to a subject in need of such treatment an effective amount of such compounds.

### BRIEF DESCRIPTION OF THE DRAWINGS

In drawings which are intended to illustrate embodiments of the invention and which are not intended to limit the scope of the invention:

FIG. 1 depicts one dimensional transient NOE difference spectra of compound S-68b in D₂O. (a) ¹H NMR spectrum. (b) Spectrum with selective irradiation of the H-4′b/H-1′a multiplet. (c) Spectrum with selective irradiation of the H-1ax/H-5ax multiplet.

FIG. 2 depicts one dimensional transient NOE difference spectra of compound R-68b in D₂O. (a) ¹H NMR spectrum. (b) Spectrum with selective irradiation of the H-4′b/H-1′b multiplet. (c) Spectrum with selective irradiation of the H-1ax/H-5ax multiplet.

FIG. 3 depicts mean plasma glucose concentrations in rats after treatment with Acarbose, Blintol, and Salacinol. Panel a): Mean plasma glucose time course following a gavage of 1000 mg/kg body weight maltose without drug (Control: ∘), or with 25 mg/kg of drug (Blintol: ●, Acarbose: ▪, Salacinol: □) n=6 per group, ±standard error. The time zero (basal) sample for each animal was calculated as the mean of the −5 and −15 minute samples. Panel b): Mean Area Under the Curve of the glucose excursion above basal, 0-90 minutes (*: P<0.005, #: P<0.05 versus Control).

FIG. 4 depicts mean plasma insulin concentrations in rats after treatement with Acarbose, Blintol, and Salacinol. Panel a): Mean plasma insulin concentration (Control: ∘, Blintol: ●, Acarbose: ▪, Salacinol: □) n=6 per group, ±standard error. Panel b): Mean Area Under the Curve of the glucose absorption rate (*: P<0.01 versus Control).

### DETAILED DESCRIPTION OF THE INVENTION

Salacinol is a naturally occurring compound which may be extracted from the roots and stems of Salacia reticulata, a plant native to Sri Lanka and India. This application relates to synthetic routes for preparing Salacinol (1), and its nitrogen (2) and selenium (3) analogues shown below. 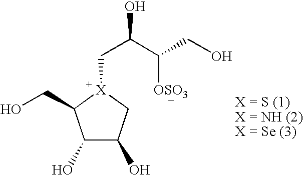
This application also relates to synthetic routes for preparing compounds (1) to (3) and stereoisomers, analogues, homologues and other derivative thereof. As used in this patent application, stereoisomers includes enantiomers and diastereoisomers. The compounds of the invention (including stereoisomers of Salacinol) comprise a new class of compounds which are not naturally occurring and may find use as glycosidase inhibitors.
1.0 Summary of General Synthetic Scheme

Scheme 1(a) below, shows the general synthetic scheme developed by the inventors for arriving at some of the target compounds. To synthesize different stereoisomers of Salacinol and its nitrogen and selenium analogues (A)-(C), 5-membered-ring sugars are reacted with sulfate-containing compounds in accordance with the invention (in Scheme 1(a) the letters (A), (B), and (C) represent all stereoisomers of Salacinol and its nitrogen and selenium analogues (1), (2) and (3) respectively). The inventors followed a disconnection approach for determining the preferred synthetic route. A reasonable disconnection is one that gives the 5-membered-ring sugars (D) since they can be synthesized easily from readily available carbohydrate precursors. Nucleophilic substitution at C₁ of the sulfate fragment (E) can then yield the target molecules (Scheme 1(a)). A potential problem with this approach is that the leaving group (L) might act later as a base to abstract the acidic hydrogens of the sulfonium salt⁷ and produce unwanted products. Therefore, the cyclic sulfate (F) may be used instead of (E) to obviate the problems associated with leaving group (L). Compound (G) may similarly be used as a cyclic sulfate reagent and is a protected version of (F).

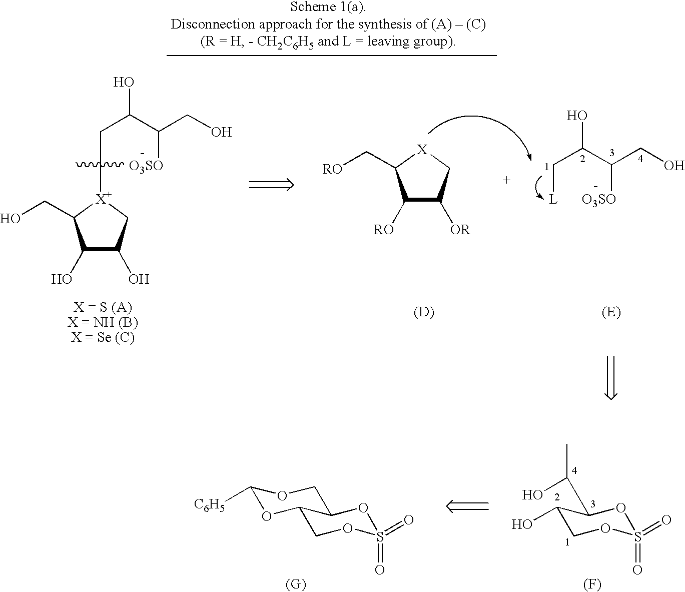

Scheme 1(b) below shows generally the coupling reactions for producing the target compounds (A)-(C). 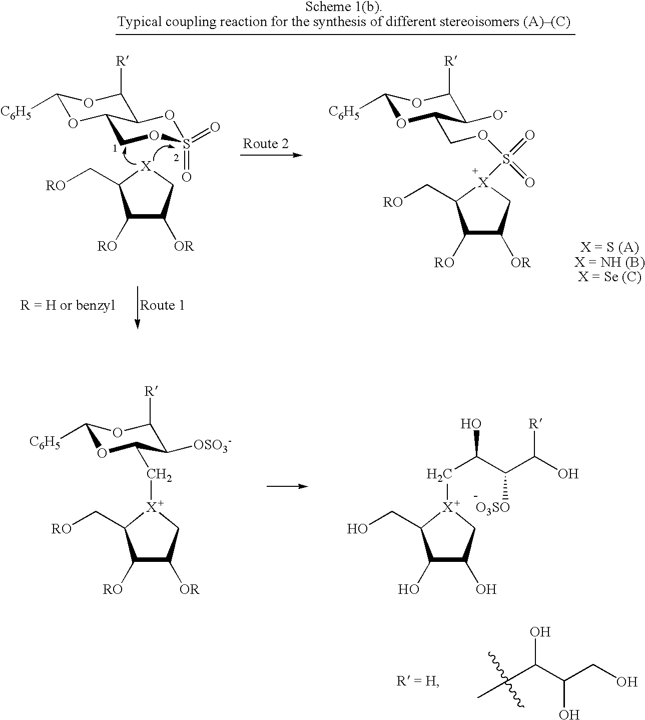
Route 1 of Scheme 1(b) shows the general strategy of reacting a cyclic sulfate with a 5-membered ring sugar to produce an intermediate compound, which may include benzyl or other protecting groups. As described in further detail below, the intermediate compound is then deprotected to yield the target compounds. The inventors have determined that Route 2 of Scheme 1 (b), a possible side reaction, does not occur.
2.0 Synthesis of Reagents

Cyclic sulfates and 5-membered-ring sugars were prepared in accordance with the synthetic schemes described below. As will be apparent to a person skilled in the art, other equivalent schemes for producing the reagents of the invention could be substituted.

2.1 Cyclic Sulfates

Cyclic sulfates were prepared in analogous fashion to the ethylidene acetal.⁸ The cyclic sulfate (7) was synthesized in 4 steps starting from D-glucose (Scheme 2). 2,4-O-Benzylidene-D-erythrithol (5) was synthesized from D-glucose in two steps,^{9,10} and then treated with thionyl chloride to yield the cyclic sulfite (6) which was oxidized to the cyclic sulfate (7) as described by Calvo-Flores et al.⁸

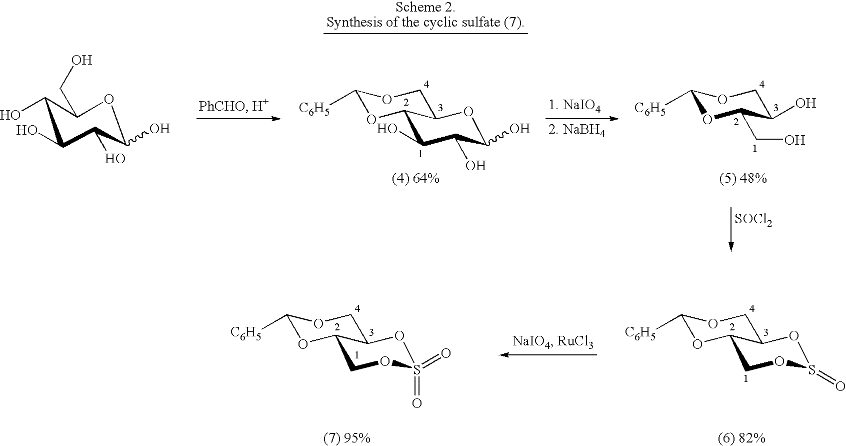

The enantiomer (10) was also synthesized using the same route but starting from L-glucose (Scheme 3). 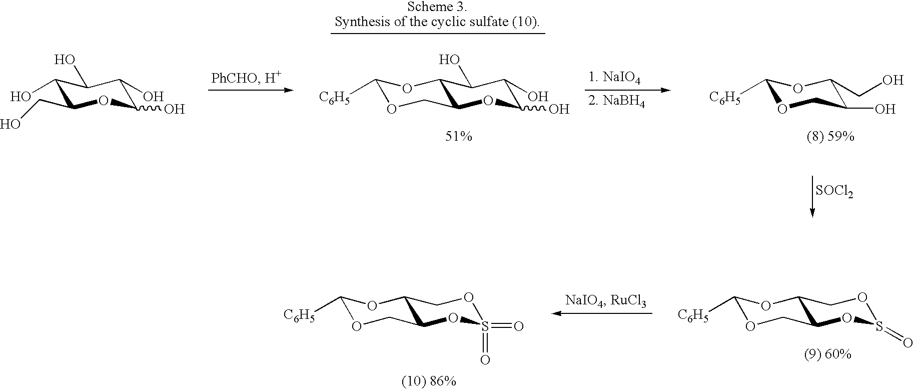
2.2 Synthesis of 5-Membered-Ring Heterocycles

In order to synthesize one of the 5-membered-ring sugars (D, X═S), 1,4-anhydro-3-O-benzyl-4-thio-D-arabinitol (11), was synthesized in 9 steps starting from D-glucose (Scheme 4).¹¹ Benzylation of the compound (11), using benzyl bromide in DMF yielded 1,4-anhydro-2,3,5-tri-O-benzyl-4-thio-D-arabinitol (12) in 90% yield. Compound (11) was debenzylated to give 1,4-anhydro-4-thio-D-arabinitol (13) in 97% yield using a Birch reduction.

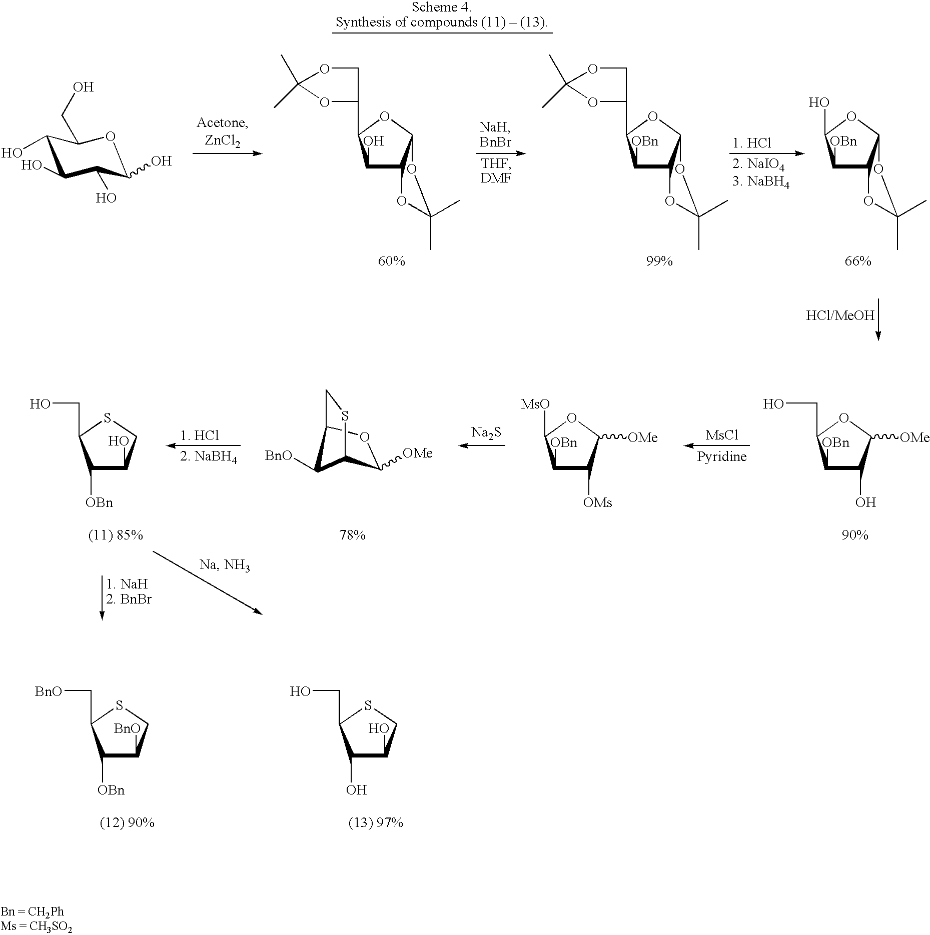

The L-isomer, 1,4-anhydro-2,3,5-tri-O-benzyl-4-thio-L-arabinitol (14) was synthesized in 5 steps starting from D-xylose (Scheme 5).¹²

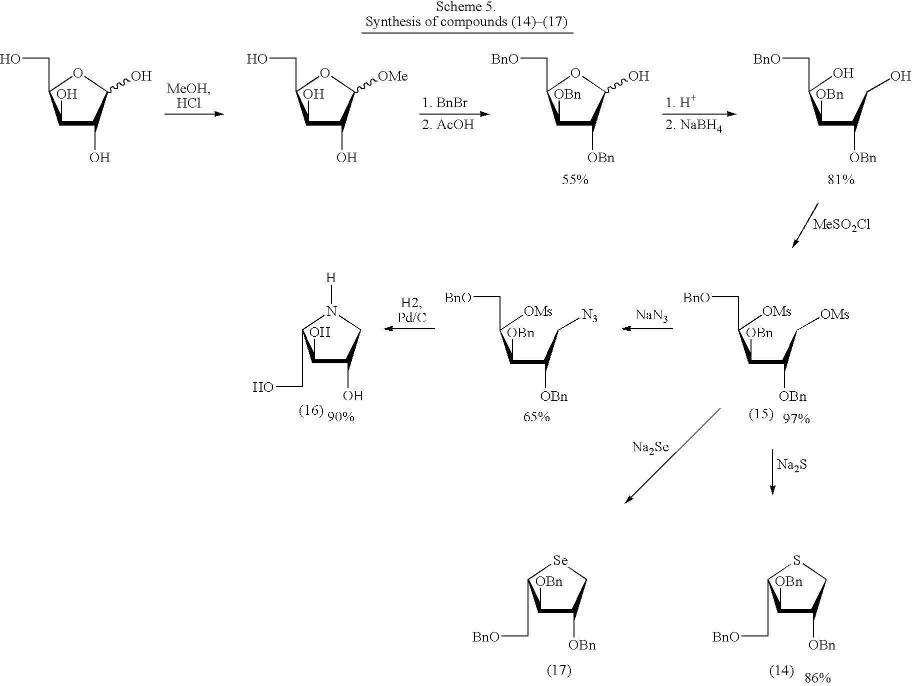

1,4-Di-O-methanesulfonyl-2,3,5-tri-O-benzyl-D-xylitol (15) is also a key intermediate for the synthesis of the aza and selena sugars (16) and (17). 1,4-Dideoxy-1,4-imino-L-arabinitol (16)¹³ was synthesized in 7 steps starting from D-xylose (Scheme 5). The enantiomer (19)¹³ was synthesized in an analogous way starting from L-xylose (Scheme 6). Compound (19) was also synthesized in 10 steps starting from D-xylose.¹³ 1,4-Anhydro-2,3,5-tri-O-benzyl-4-seleno-D-arabinitol (20) was synthesized in 5 steps starting from L-xylose (Scheme 6). To synthesize compound (20), Na₂Se was made in-situ by treatment of selenium metal with sodium in liquid ammonia.

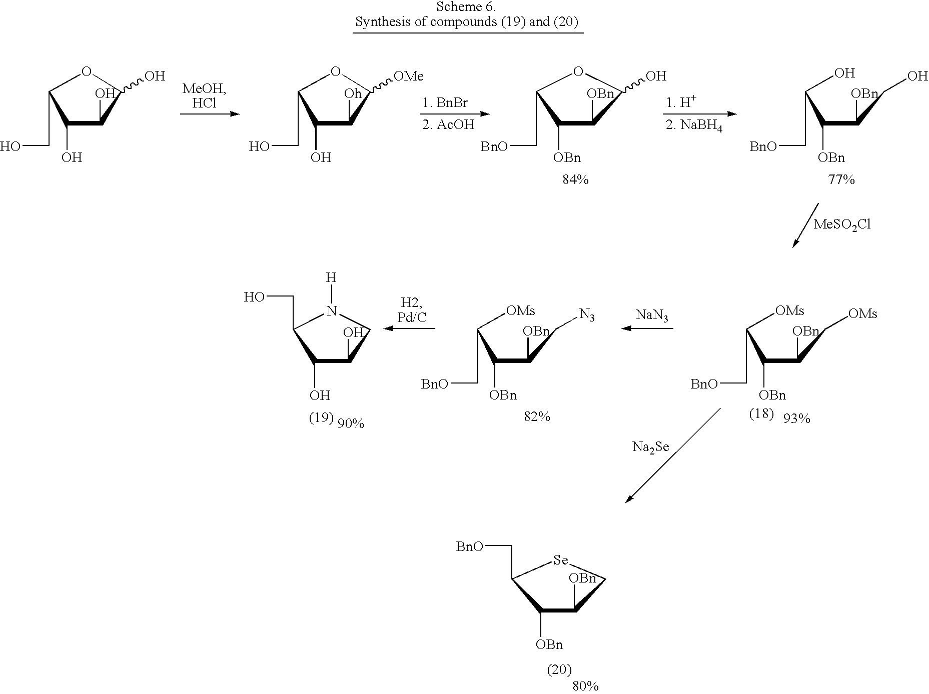

Scheme 6(a) below shows a more generalized scheme for synthesizing compound (20) using other possible protecting groups (R═COR, CH₂C₆H₄—OMeₚ). 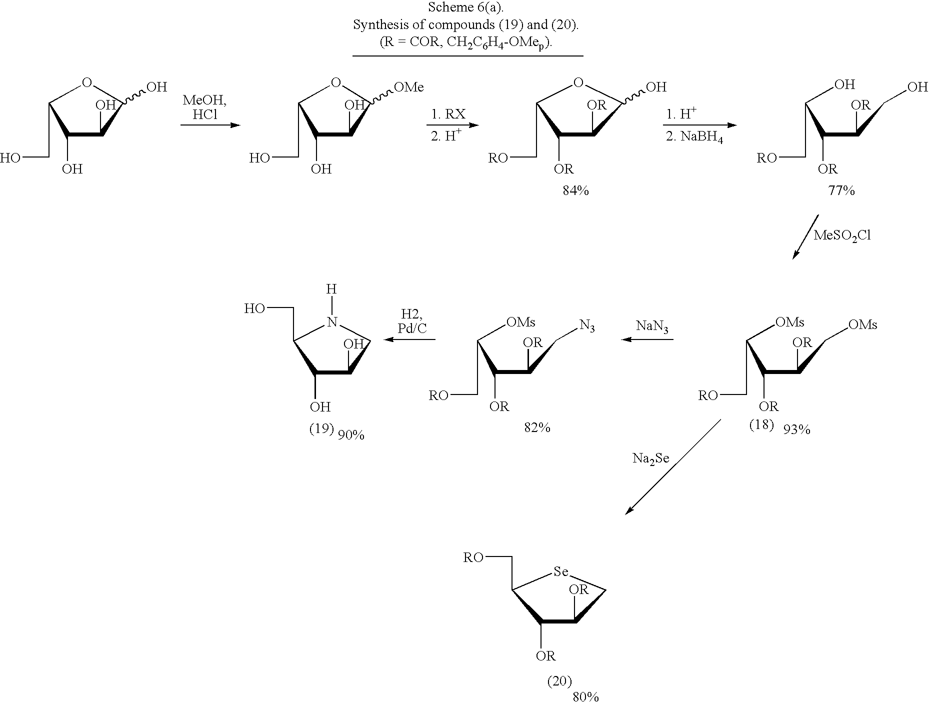
3.0 Synthesis of Target Compounds (1)-(3)

The target compounds (1)-(3) were prepared by opening of the cyclic sulfates by nucleophilic attack of the heteroatoms on the 5-membered rings (Scheme 1(b) above). The heteroatom gives rise to a positively charged cation and the cyclic sulfate gives rise to a negatively charged counterion. This internal salt structure may explain the stability of the target compounds toward decomposition by further nucleophilic attack.

3.1 Synthesis of Salacinol

Salacinol (1) was synthesized by nucleophilic substitution of the protected thio-arabinitol (12) with the cyclic sulfate (10) (1.2 equiv) in dry acetone containing K₂CO₃, to give the protected intermediate compound (21) in 33% yield. Hydrogenolysis of the benzyl and benzylidene groups in AcOH:H₂O, 4:1 afforded Salacinol (1) in 67% yield (Scheme 7).

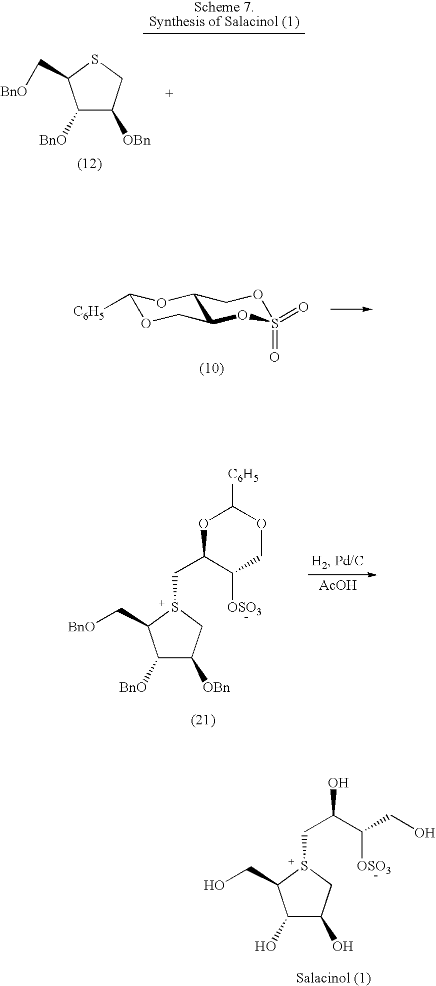

The same procedure was used to prepare intermediate compound (22) in 79% yield from the enantiomeric cyclic sulfate (7). Deprotection as before gave compound (23) in 59% yield (Scheme 8). Compound (23) is a diastereomer of Salacinol (1).

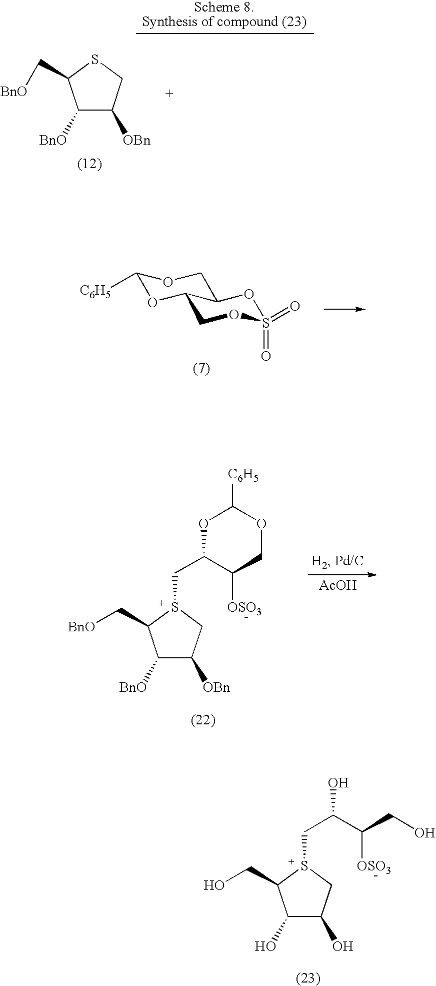

Compound (24) was prepared in 40% yield from (7) and the enantiomeric thio-ether (14) (Scheme 9). Deprotection in 80% yield gave the enantiomer of Salacinol (25).

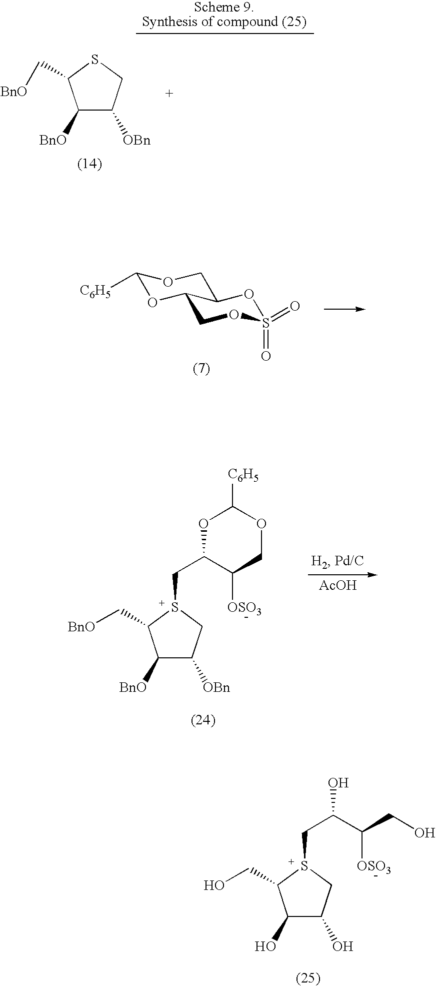

To reduce the number of synthetic steps, the inventors attempted the coupling reactions with the deprotected thio-arabinitols. Thus, the partially deprotected compound (11) was reacted with the cyclic sulfate (10) in acetone, to give compound (26) in 32% yield. Deprotection yielded Salacinol (1) in 36% yield (Scheme 10).

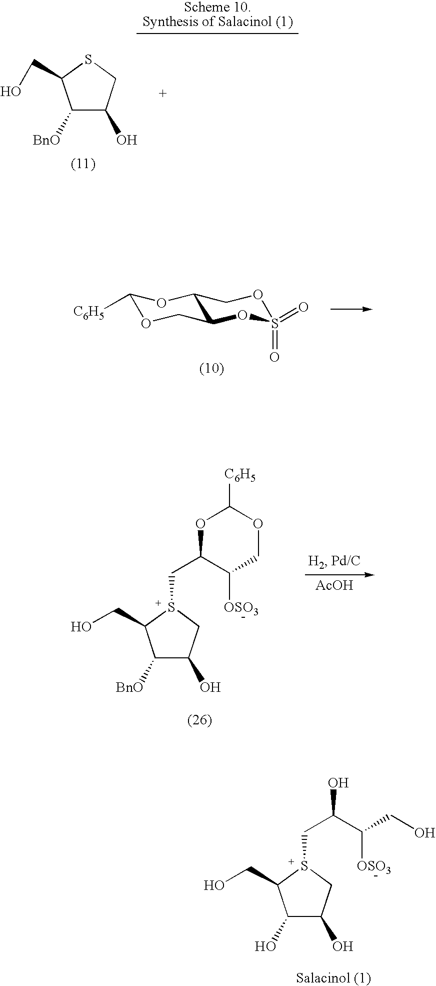

The fully-deprotected thio-arabinitol (13) was not soluble in acetone and the reaction in methanol produced several products.

3.1.1 Alternative Synthesis of Salacinol

As described above, a key step in the published syntheses of Salacinol (1)^{15,25} is the ring opening reaction of a cyclic sulfate by nucleophilic attack of the ring sulfur atom of 1,4-anhydro-4-thio-D-pentitol (33) (Scheme 10a). The alkylation reaction involving these reagents is dependent on the protecting groups on the cyclic sulfate. Thus, the unoptimized reaction of the per-benzylated thioether 33 with the benzylidene-protected cyclic sulfate 34 in acetone, containing potassium carbonate, proceeded in 33% yield (Scheme 10a).²⁵ A similar yield was obtained in the reaction with the monobenzylated thioether 36.²⁵ Reaction of the unprotected thioether 38 with the isopropylidenated-cyclic sulfate 39 in DMF proceeded in 61% yield to give 40, although its reaction with the corresponding benzylated-cyclic sulfate 41 did not proceed.¹⁵ The latter derivative 41 is clearly a much less reactive alkylating agent than 39. Significant decomposition of the cyclic sulfates 39 and 41 at temperatures of 60-70° C. in DMF was also observed.¹⁵ Deprotection of 40 proceeded in 75% yield to afford Salacinol 1 in 46% overall yield.¹⁵

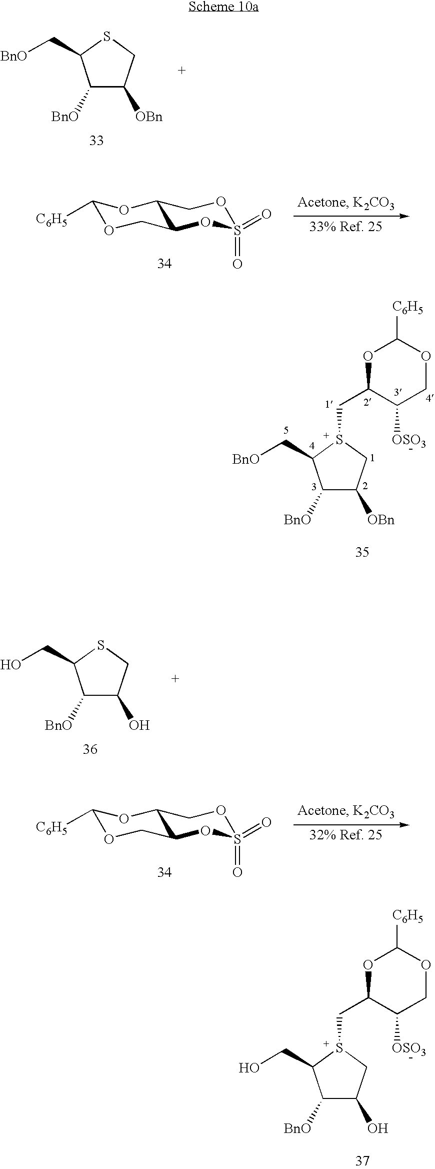

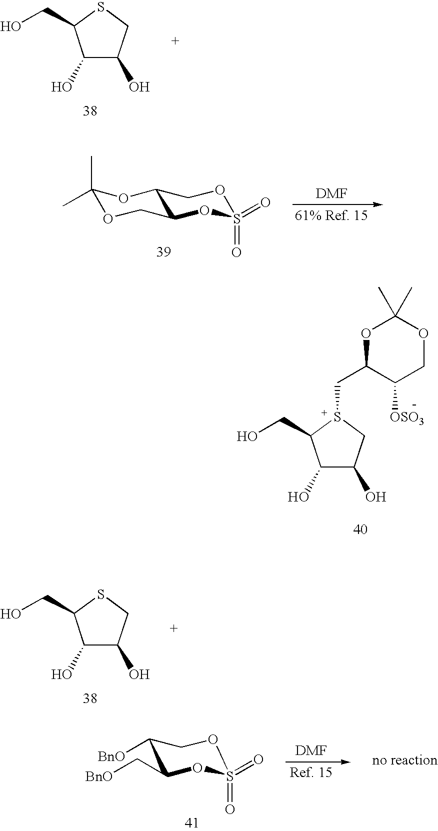

The biological importance of Salacinol (1)^{1,2,27} prompted the inventors to investigate a more efficient method for its synthesis. The Hughes-Ingold rules indicate that the S_{N}2 reaction between a neutral nucleophile, such as 33 or 36, and a neutral electrophile, such as 34, 39 or 41, should show a large increase in rate on increasing solvent polarity. 1,1,1,3,3,3-Hexafluoroisopropanol (HFIP) has a higher normalized Dimroth-Reichardt solvent polarity parameter, E_{T}^{N}=1.068, than water, E_{T}^{N}=1.00. In contrast, the E_{T}^{N} values for acetone and DMF are only 0.355 and 0.404, respectively. Furthermore, HFIP, bp=59° C., is volatile, thus facilitating product purification. Preliminary studies indicated that tetrahydrothiophene reacted cleanly with 34 and 41 in HFIP at 45° C. for 2 days to give the desired alkylation products in >90% yield.

Therefore, a systematic evaluation of the role of solvent in the alkylation reactions of 33 with benzyl- or benzylidene-protected cyclic sulfates 41 or 34, respectively was undertaken. The reactions were carried out in acetone and hexafluoroisopropanol (HFIP) concurrently under identical conditions of concentration, temperature, and duration (Scheme 10b). Reaction of the thioether 33 (1 equiv) and the cyclic sulfate 41 (1.2 equiv) in acetone containing K₂CO₃ at 75-80° C. in a sealed tube proceeded very slowly and yielded the desired alkylated product 42 in only 5% yield; the remainder of the starting materials was recovered. Prolonged heating and use of excess cyclic sulfate did not improve the yields. In addition, when excess cyclic sulfate 39 was used, its slow decomposition complicated the purification of the product 42 formed. However, the analogous reaction between 33 and the cyclic sulfate 41 in HFIP yielded the adduct 42 in 45% yield, with recovery of the unreacted starting materials (Scheme 10b). It is noteworthy that the analogous reaction between 33 and the cyclic sulfate 41 in the polar, protic solvent 2-propanol at 83° C. for 26 h did not yield any desired product, the starting materials being recovered. It would appear, therefore, that it is the highly polar nature of HFIP that is important in facilitating this reaction.

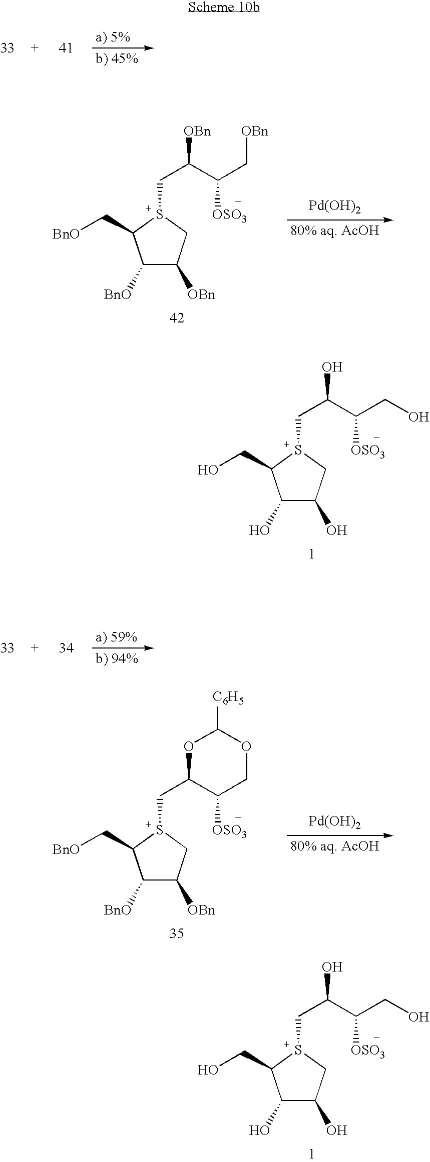

Some studies¹⁵ have indicated a far lesser reactivity of the benzylated cyclic sulfate relative to the cyclic sulfate containing an acetal protecting group (Scheme 10a). Thus, the reactions of the benzylidene-protected cyclic sulfate 34 in acetone and HFIP, containing potassium carbonate, under identical conditions of concentration, temperature, and duration were examined next (Scheme 10b). The alkylation reaction of 33 with 34 in acetone proceeded with a dramatic increase in the yield (59%) of the alkylated product 35 relative to the reaction with 41. The improvement from the unoptimized yield of 33%²⁵ is due to the use of a more concentrated reaction mixture.

More significantly, the desired product 35 was obtained in 94% yield when the reaction was performed in HFIP. Higher temperatures (>80° C.) and prolonged reaction times led to the decomposition of the cyclic sulfate, although the stability of the cyclic sulfate was greater in the presence of K₂CO₃. The increased yields in HFIP may be accounted for by better solvation of the transition states for the reactions and of the adducts. The increased reactivity of the cyclic sulfate with the benzylidene protecting group (34) may be accounted for by the relief of ring strain accompanying the reaction, unlike in the corresponding reaction of the benzyl-protected cyclic sulfate 41. Finally, the reaction of the thioether 38 (not containing protecting groups) with the benzylidene-protected cyclic sulfate 34 in HFIP was examined. At 60° C., decomposition of the cyclic sulfate was observed, with no significant formation of the desired coupled product. Hydrogenolysis of the protected derivatives 35²⁵ and 42 afforded Salacinol (1), although this step was problematic because of poisoning of the catalyst, and only afforded the product in 65% yield. The sterochemistry of Salacinol (1) shown in Scheme 10(b) is an equivalent representation to that shown on page 2 hereof.

In order to obviate the problematic hydrogenolysis step, the inventors next chose to examine the reaction of the thioether containing p-methoxybenzyl ether protecting groups with the benzylidene-protected L-erythritol-1,3-cyclic sulfate; the inventors reasoned that the removal of all protecting groups by acid hydrolysis would be facile. Thus, 2,3,5-tri-O-p-methoxybenzyl-1,4-anhydro-4-thio-D-arabinitol (43), synthesized in 87% yield from 38, was reacted with the cyclic sulfate 34 in HFIP to afford the sulfonium salt 44 in quantitative yield (Scheme 10c). Deprotection of 44 proceeded smoothly (86%) in aqueous trifluoroacetic acid to afford Salacinol 1 in 75% overall yield. The latter sequence represents, therefore, an efficient synthesis of the biologically important natural product Salacinol 1.

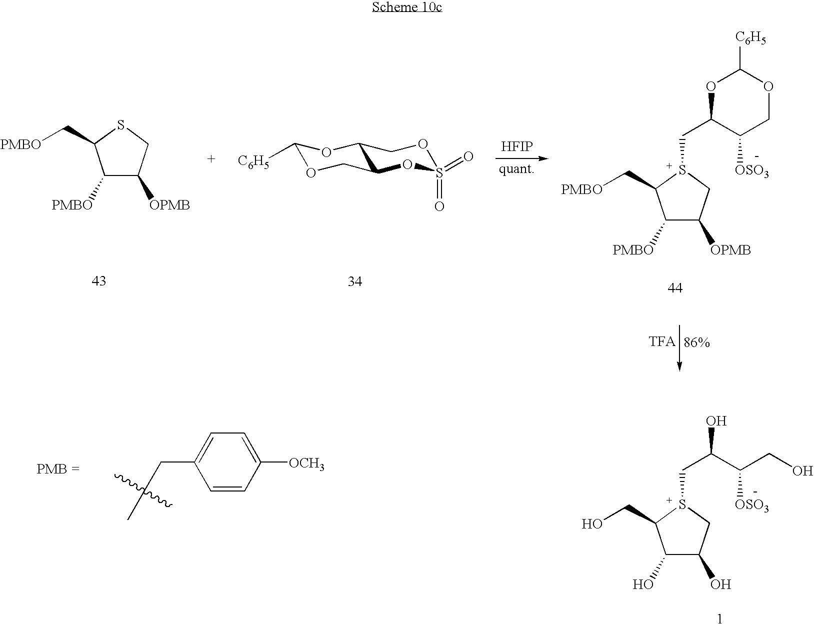

The inventors considered the stereochemistry at the stereogenic sulfonium center in 35, 42, and 13 and determined that these reactions proceeded stereoselectively irrespective of the solvent used in the reaction. The stereochemistry was confirmed by means of NOESY experiments that showed clear correlations between H-4 and H-1′, thus indicating the presence of the isomer with a trans relationship between C-5 and C-1′. The barrier to inversion at the sulfonium ion center must be substantial since no evidence for isomerization in these and related derivatives²⁹ has been noted.

3.2 Synthesis of Selenium Analogues

The seleno-analogue intermediate (27) (R═CH₂C₆H₅) was made starting from the seleno-arabinitol (20) (R═CH₂C₆H₅) and the cyclic sulfate (10) in excellent yield 86% (Scheme 11), but NMR spectroscopy showed the presence of two isomers in a ratio of 7:1 that differed in stereochemistry at the stereogenic selenium center. The isomers were separable by analytical HPLC. The inventors have assigned the name “Blintol” to the new selenium analogue (3).

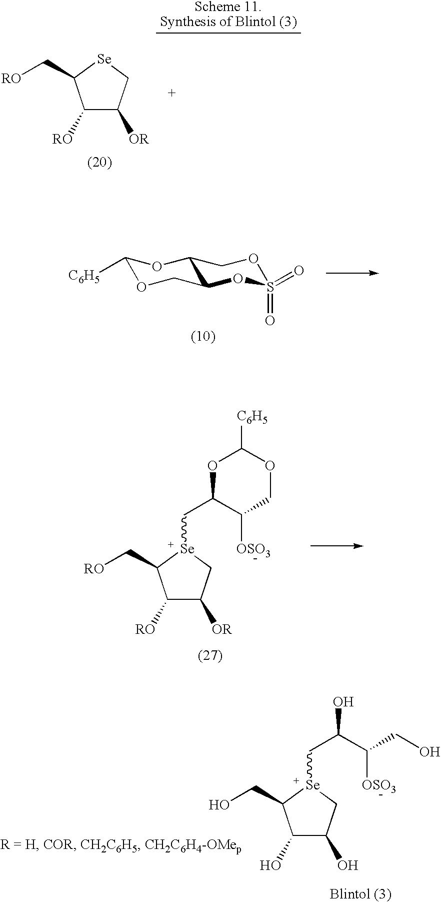

The seleno-analogue intermediate (28) (R═CH₂C₆H₅) was made starting from the seleno-arabinitol (20) (R═CH₂C₆H₅) and the cyclic sulfate (7) in excellent yield 97% (Scheme 12); a mixture of two isomers in a ratio of 3:1 that differed in stereochemistry at the stereogenic selenium center was obtained. The isomers were separable by analytical HPLC. 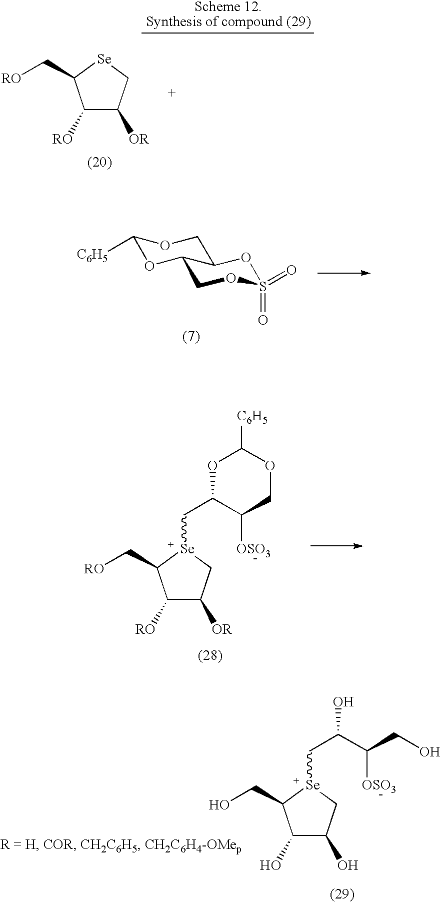
Compound (29) is a diastereomer of Blintol (3).
3.2.1 Alternative Route to Synthesis of Blintol

Retrosynthetic analysis indicated that Blintol (3) could be obtained by alkylation of anhydroseleno-D-arabinitol (45) at the ring heteroatom using an appropriately protected cyclic sulfate (47) (Scheme 12a).²⁵

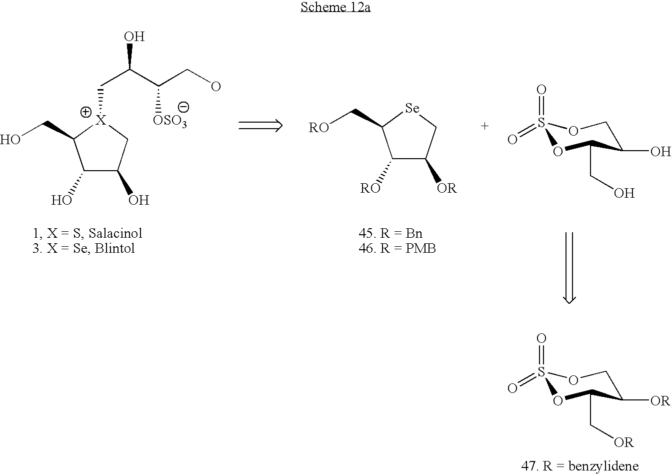

The previously discussed synthesis of Blintol (3) used benzyl ethers as the protecting groups for the hydroxyl groups on the anhydroseleno-D-arabinitol 45.²⁶ However, the deprotection of the benzyl-protected Blintol (3) by hydrogenolysis was problematic due to the poisoning of the palladium catalyst by small amounts of the selenoether 45 formed in the reaction mixture.

In order to eliminate the problematic hydrogenolysis step, the use of p-methoxybenzyl (PMB) protecting groups on the seleno-D-arabinitol, as in the inventors' optimized synthesis of Salacinol (1),⁵³ was considered. Thus, the reaction of the p-methoxybenzyl-protected selenoether 46 with the benzylidene-protected L-erythritol-1,3-cyclic sulfate (47; R=benzylidene) was examined. Since both PMB and benzylidene protecting groups are labile to acidic hydrolysis, the removal of all protecting groups by acid hydrolysis is facile.⁵³

The synthesis of the PMB-protected anhydroseleno-D-arabinitol (45) from L-xylose (48) required the judicious choice of aglycon. Initial attempts to use the allyl glycosides yielded an inseparable mixture of the desired allyl xylofuranosides and undesired allyl xylopyranosides. Furthermore, the cleavage of the allyl group was judged to be too expensive a process for large-scale synthesis. Nevertheless, the mixture of furanosides and pyranosides was used in the successful synthesis of Blintol (3), their separation being effected at a later stage in the synthetic scheme.

These concerns led the inventors to explore the following strategy: 1) The use of n-pentenyl glycosides, first exploited by Fraser-Reid and coworkers;⁵⁴ this group was also reported to be cleaved by NBS without affecting the PMB groups,⁵⁵ and 2) The use of boric acid in the acid-catalyzed acetylation of L-xylose (48) to improve the furanoside to pyranoside ratio.⁵⁶ The latter procedure led to the conversion of L-xylose (48) to 1,2,3,5-tetra-O-acetyl-D-xylofuranose (49) in a two-step, one-pot procedure. Analysis of the ¹H and ¹³C NMR spectra indicated that the furanosides 49 were formed exclusively without formation of the undesired pyranoside side products (Scheme 12b).

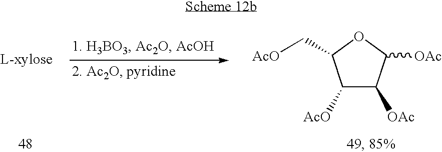

Compound 49 was then treated with 4-penten-1-ol and BF₃ OEt₂ to give the 4-pentenyl 2,3,5-tri-O-acetyl-L-xylofuranosides (50).⁵⁷ This compound underwent acidic hydrolysis to cleave the acetyl groups, followed by the reprotection of the three hydroxyl groups with PMB groups, to afford the 4-pentenyl 2,3,5-tri-O-p-methoxybenzyl-L-xylofuranosides (52). The anomeric hydroxyl group of 52 was then released using NBS in acetonitrile-water to yield the corresponding 2,3,5-tri-O-p-methoxybenzyl-L-xylofuranose (53) (Scheme 12c).

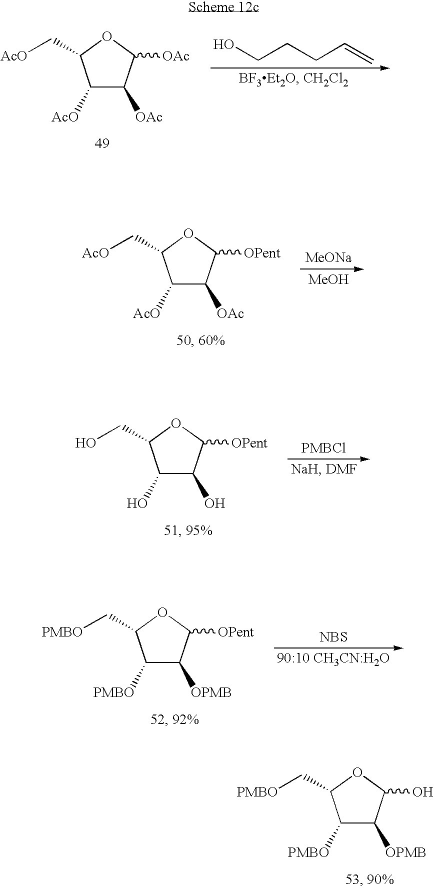

The 2,3,5-tri-O-p-methoxybenzyl-L-xylofuranose 53 was reduced to the corresponding xylitol 54 by NaBH₄; mesylation of the hydroxyl groups then gave the dimesylate 55. Compound 55 was then converted to the 1,4-anhydro-2,3,5-tri-O-p-methoxybenzyl-4-seleno-D-arabinitol (56) in 83% yield, using sodium selenide, generated in situ, from selenium metal and sodium borohydride in ethanol (Scheme 12d).

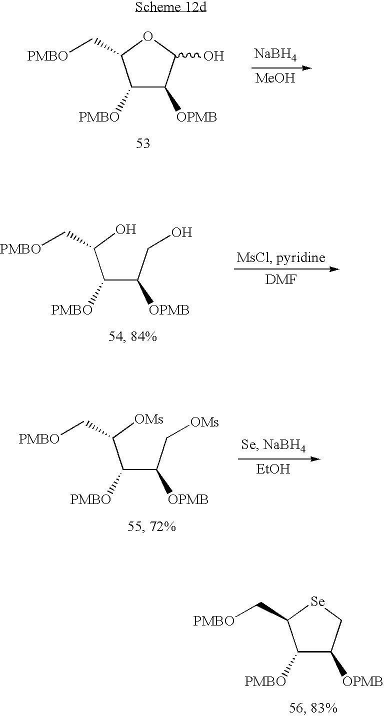

Another factor in the synthesis of Blintol (3) (and the optimized synthesis of Salacinol⁵³) is the availability of 2,4-O-benzylidene-L-erythritol-1,3-cyclic sulfate (57). This compound was previously prepared from L-glucose.²⁵ However, due to the high cost of L-glucose and the fact that it was the starting material in a six-step synthetic route, it was desirable to prepare the cyclic sulfate (57) from a less expensive material. As described herein the inventors have successfully prepared the cyclic sulfate 57 from D-glucose (58).

Using the method developed by the inventors,^{25,26} the benzyl-protected cyclic sulfate 62 was prepared from D-glucose (58). It is interesting to note that cleavage of the benzylidene protecting group in compound 59 was achieved with 60% TFA at room temperature for 30 min to afford the corresponding diol 60 in a comparable yield to that obtained with aqueous acetic acid. Since the original method involved refluxing compound 59 in 80% HOAc for 48 h, this modification proved to be more efficient. Compound 62 underwent hydrogenolysis to afford the unprotected cyclic sulfate 63. Installation of the benzylidene acetal using pyridinium p-toluenesulfonate (PPTS) as the catalyst was the important step since, under these conditions, the cyclic sulfate was not cleaved. The desired benzylidene-protected cyclic sulfate 57 was obtained in 71% yield (Scheme 12e).

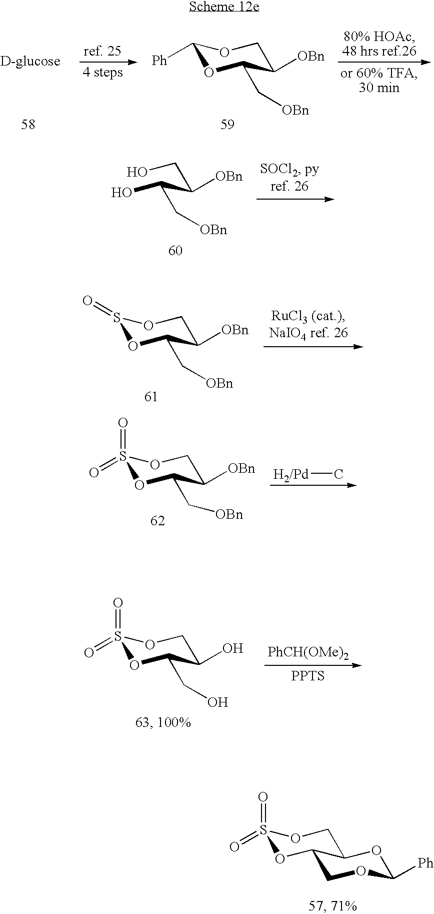

The coupling reaction of the anhydroseleno-D-arabinitol 56 with the cyclic sulfate 57 in 1,1,1,3,3,3-hexafluoro-2-propanol (HFIP) at 60-65° C. proceeded smoothly in 7 h, to give a mixture of the 2,3,5-tri-O-p-methoxybenzylselenonium salts 64 in 95% yield (Scheme 12f). Analysis of the ¹H and ¹³C NMR spectra indicated that compound 64 was a 7:1 mixture of isomers at the stereogenic selenium center. The major isomer was assigned to that with a trans relationship between C-5 and C-1′, by analogy with the results obtained previously.²⁶

The selenonium salts 64 were subsequently deprotected by treatment with trifluoroacetic acid (TFA), and purified by recrystallization to afford pure Blintol (3) in 62% yield (Scheme 12f). 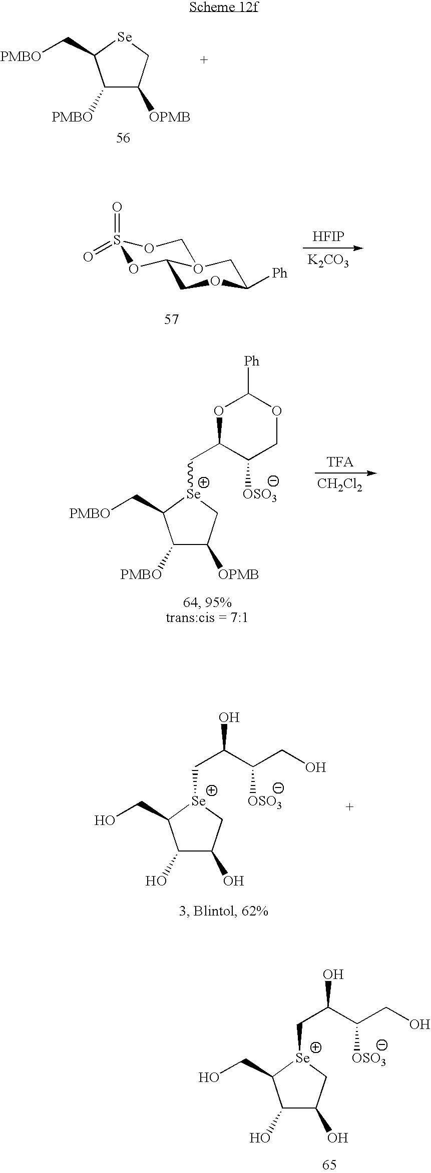
3.3 Synthesis of Nitrogen Analogues

The nitrogen analogue intermediate (30) was made by the reaction of the deprotected imino-arabinitol (19) with the cyclic sulfate (10) in a good yield 72% (Scheme 13). Compound (19) was not soluble in acetone so the reaction was performed in dry methanol. A side product (19%) which was identified to be the product of methanolysis of the cyclic sulfate was obtained. The inventors have assigned the name “Ghavamiol” to the new nitrogen analogue (2). Compound (30) was deprotected to give Ghavamiol (2) in 64% yield.

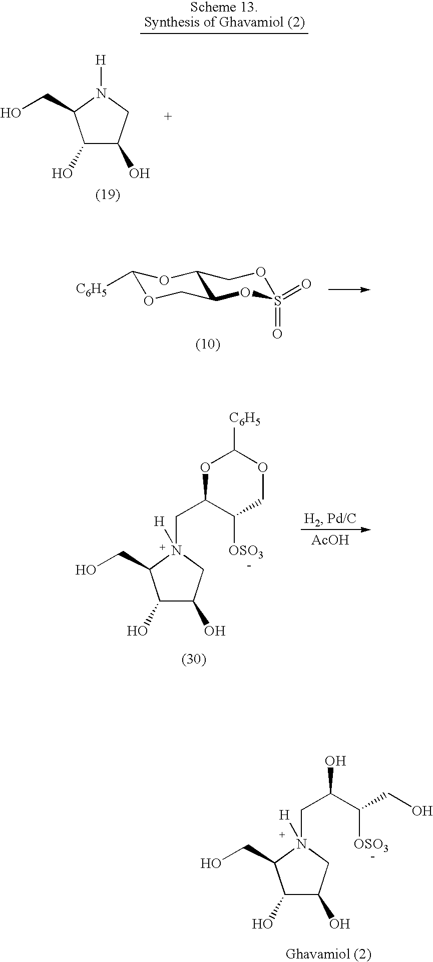

The enantiomer intermediate (31) was made by the reaction of the deprotected imino-arabinitol (16) with the cyclic sulfate (7) in a good yield 72% (Scheme 14). A side product (21%) which was identified to be the product of methanolysis of the cyclic sulfate was obtained. Compound (31) was deprotected to give compound (32) in 77% yield. Compound (32) is the enantiomer of Ghavamiol (2). 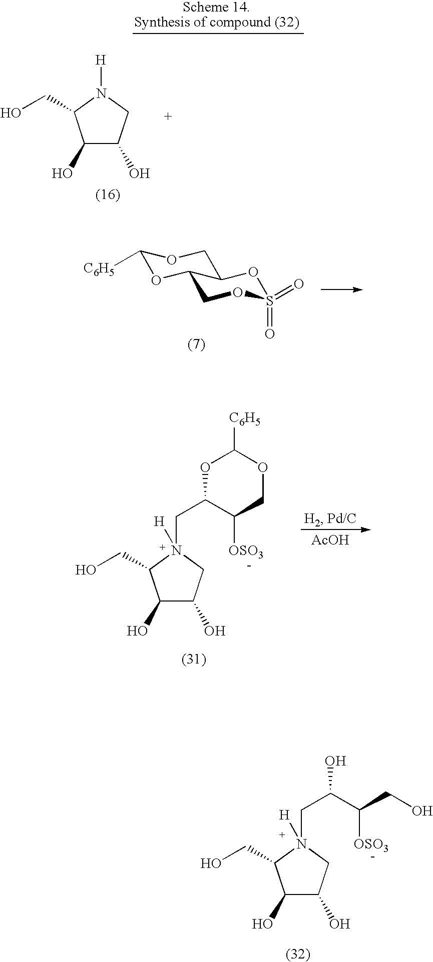
4.0 Alternative Synthetic Schemes
4.1 Six-Membered Ring Analogues

In an alternative embodiment of the invention, target compounds having potential application as glycosidase inhibitors may be synthesized in the manner described above using 6-membered rather than 5-membered ring heterocycles as reagents. As in the embodiments described above, the cyclic sulfate (described above) is opened in the coupling reaction due to nucleophilic attack of the heteroatoms (i.e. X═S, Se, NH) on the ring sugars. As will be apparent to a person skilled in the art, the general formulas for the 6-membered sugar reagent and resulting target compound are as shown below. 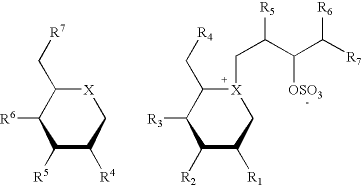
The 6-membered ring target compound shares the same internal salt structure as the 5-membered ring embodiment. The substituent groups may vary as described below without departing from the invention.

In particular, in order to expand the repertoire of molecules of this class that could serve as glycosidase inhibitors, the inventors proposed to synthesize N-alkylated 1,5-dideoxy-1,5-iminoxylitol (66a) and deoxynojirimycin (67a) having the same L-erythritol-derived, sulfated side-chain as Salacinol. The advantage of having an internal sulfate counterion for the ammonium salt was deemed to be worth pursuing in order to investigate whether such a structural modification would lead to increased in-vivo stability and/or membrane permeability. In addition, the internal sulfate salt and polar side-chain may provide cationic inhibitors that bind to glycosidase enzymes without deprotonating the catalytic active-site carboxylic acid and provide additional insight into the structural features that are important for inhibition. The inventors describe herein the syntheses of 66a and 67a as well as the corresponding sulfonium and selenonium analogues 68a, 69a and 70a. The inventors report also the syntheses of the corresponding enantiomers or diastereomers 66b-70b resulting from incorporation of a side chain derived from D-erythritol.

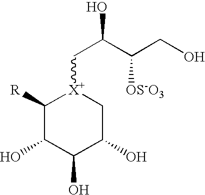

| | |
|---|---|
| 66a | X = NH R = H |
| 67a | X = NH R = CH₂OH |
| 68a | X = S R = H |
| 69a | X = S R = CH₂OH |
| 70a | X = Se R = H |

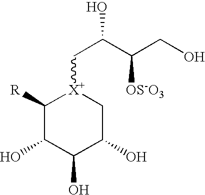

| | |
|---|---|
| 66b | X = NH R = H |
| 67b | X = NH R = CH₂OH |
| 68b | X = S R = H |
| 69b | X = S R = CH₂OH |
| 70b | X = Se R = H |

Each target six-membered ring compound was synthesized in two stereoisomeric forms (a or b) by using either of the enantiomeric forms of the cyclic sulfate 71a or 71b as the source of the sulfated alkyl side chain. In the case of compounds 66, 68, and 70 these stereoisomers are enantiomers while compounds 67 and 69 were prepared as either of two diastereomers. For the enantiomeric sulfonium salts 68a and 68b, R/S isomers at the stereogenic sulfonium-ion center were separated and characterized independently. Similar isomers for the sulfonium salts 69 and selenonium salt 70 were not separable by chromatography and the products were characterized as mixtures. In the case of the ammonium salts 66 and 67, inversion at the nitrogen center, via the free amine, was sufficiently fast in solution at room temperature that stereoisomers at the ammonium center were not observed.

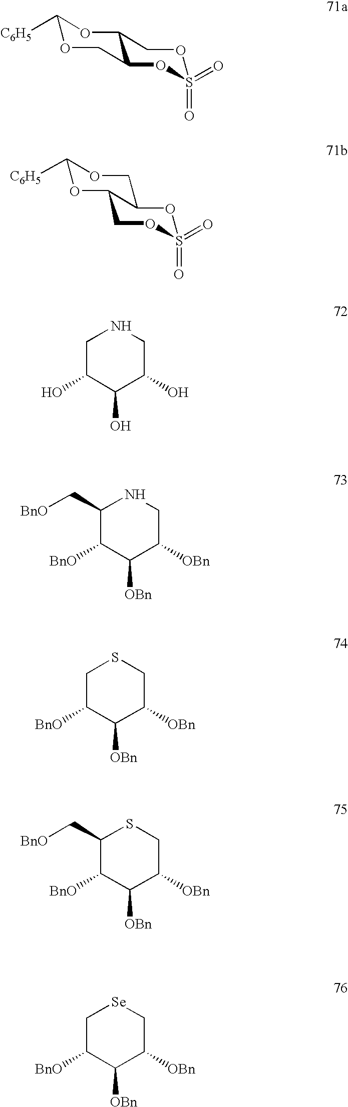

The general synthetic strategy (Scheme 15) involved alkylation of the piperidine (72 and 73), tetrahydrothiapyran (74 and 75), or tetrahydroselenapyran (76) heterocycles with either the 2,4-O-benzylidene-L-1,3-cyclic sulfate (71a),^{16,53} derived from L-glucose, or its enantiomer (71b), ^{16,53} obtained from D-glucose. In general, the reactions with the less-expensive 71b were examined first. These methods are analogous to those described above used by the inventors to synthesize the five-membered ring analogues, Salacinol and its nitrogen or selenium congeners.^{16,25,26, 53,72} 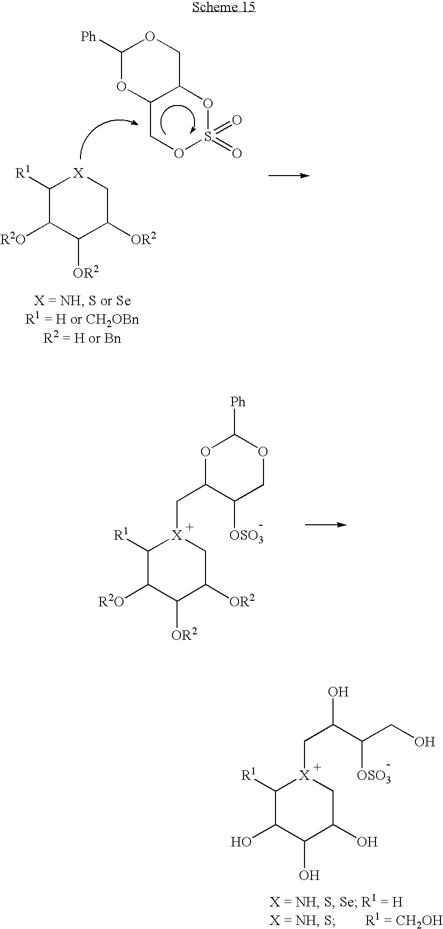
4.1.1 Preparation of Starting Materials

In preliminary experiments investigating the reactivity of the cyclic sulfate 71b, the inventors found that, for complex amine nucleophiles having only secondary alcohols as additional functional groups, protection of hydroxyl groups was unnecessary, but that any primary alcohol functional groups may be alkylated in competition with amines.

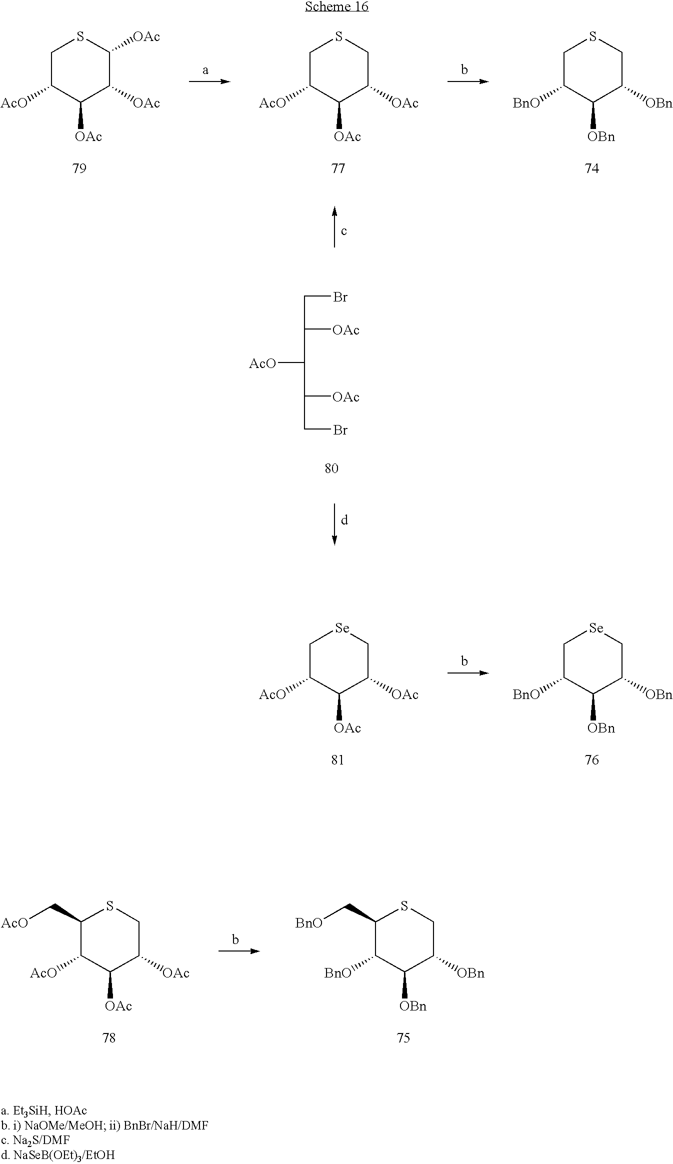

Accordingly, the unprotected anhydroxylitol imine (72) was prepared by the literature method⁷⁵ while deoxynojirimycin was prepared as its tetra-O-benzyl derivative (73).⁷³ The tetrahydrothiapyran derivative 74 was prepared (Scheme 16) by deacetylation and benzylation of the known tri-acetate 77.⁷⁵ The benzylated tetrahydrothiapyran 75 was similarly prepared from the known anhydro-5-thio-D-glucitol tetra-acetate (78)²⁸ by protecting group interchange. Compound 77 was obtained, in turn, either by reduction of tetra-O-acetyl-5-thio-D-xylopyranose (79)³⁹ or, more conveniently, from reaction of acetylated 1,5-dibromoxylitol (80) with sodium sulfide.⁷⁵ The selenium heterocyle 81 was prepared by substituting NaSeB(OEt)₃ (obtained in situ⁷⁷ by reduction of Se with NaBH₄/EtOH) for sodium sulfide in the reaction with acetylated 1,5-dibromoxylitol (80) (Scheme 16). Subsequent exchange of the acetates for benzyl protecting groups gave the desired tetrahydroselenapyran derivative 76, whose preparation has been reported by an unrelated method.⁷⁸

4.1.2 Target Ammonium Compounds

Compound 72 was reacted with the D-cyclic sulfate 71b in MeOH containing K₂CO₃ (Scheme 17). Isolation of the more polar product gave the ammonium salt 84 in 43% yield. An abundant side-product (83) resulting from opening of the cyclic sulfate by the methanol solvent could be isolated from the early chromatographic fractions. A similar reaction with the L-cyclic sulfate 71a gave somewhat less of this side product and the desired coupled product 82 was obtained in slightly higher yield (56%). The ¹H NMR spectra of compounds 82 and 84 exhibited sharp resonances for methylene groups α to the amine in D₂O (made basic with K₂CO₃), but neutral or acidic D₂O solutions gave downfield shifts and much broader resonances for these methylene resonances. The inventors attribute these observations to exchange, at an intermediate rate relative to the chemical-shift NMR time scale, of the conjugate-acid R/S ammonium salts, with nitrogen inversion taking place via the free amines that exist in equilibrium with their conjugate acids at acidic pH.

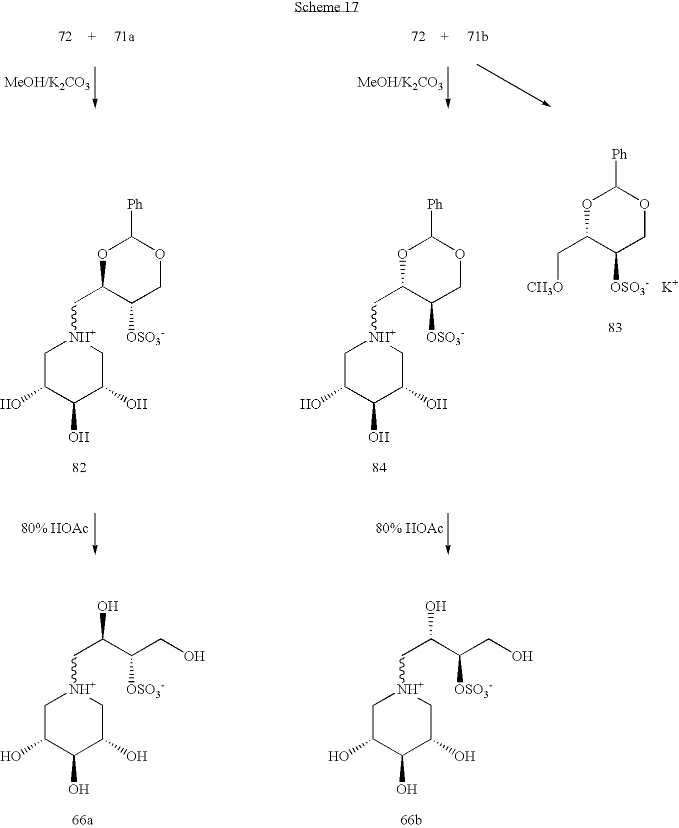

Removal of the benzylidene protecting groups by hydrolysis in aqueous acetic acid gave the target compounds 66a (73%) and 66b (72%) after purification by chromatography on silica gel. These ammonium salts gave severely exchange-broadened NMR spectra and were more productively characterized by adding base to the NMR samples to produce the conjugate amine bases. Prolonged treatment with strong base should be avoided, however, due to the possibility of sulfate ester hydrolysis, as noted below. As expected for enantiomers, the NMR data for 66a and 66b were virtually identical although small differences in chemical shifts between different samples for both identical and enantiomeric compounds were noted. These differences were attributed to the concentration and temperature dependence of the NMR chemical shifts between samples. The tendency of zwitterionic compounds to exist as aggregates in solution is the likely origin of these effects.

The coupled products 85 and 86, derived from the benzyl-protected deoxynojirimycin, were obtained by reaction of compound 73 with the cyclic sulfates 71a and 71b in acetone/K₂CO₃ in yields of 80% and 65%, respectively (Scheme 18). The ¹H NMR resonances for compounds 85 and 86 were extremely broad in CDCl₃ but sharpened in CD₃OD (made basic with NaOD), thus indicating that the coupled products were obtained as an equilibrating mixture of the desired ammonium salts with the corresponding conjugate bases. Simultaneous removal of both the benzyl and benzylidene protecting groups was achieved by hydrogenolysis in aqueous acetic acid to give the target compounds 67a and 67b.

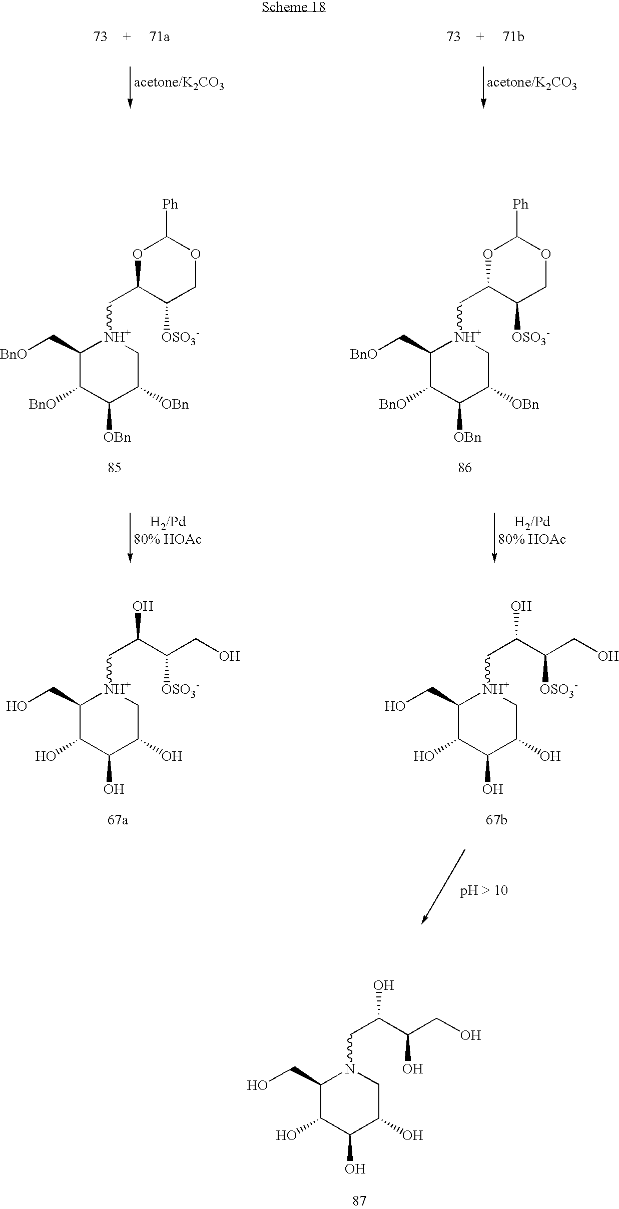

Analysis by ¹H NMR spectroscopy indicated that these products were contaminated by KOAc. Nevertheless, other than a resonance at δ 1.8 in the spectrum that was attributed to the acetate impurity, the target compounds were essentially pure and all resonances in both the ¹H and ¹³C spectra were assigned by two-dimensional techniques. Prolonged storage of the NMR sample of compound 67b in D₂O/NaOD at pH>10 produced a slow loss of the 3′-sulfate group as evidenced by an upfield shift of the H-3′ resonance. After 2 days at ambient temperature the sulfate ester had been completely hydrolyzed to yield cleanly the tertiary amine compound 87 and inorganic sulfate salts.

The ¹H NMR data for all of the amine compounds in D₂O (pH>8) indicated that the predominant conformation of the piperidine ring was ⁴C₁ (carbohydrate numbering) and that this conformational preference did not appear to change upon protonation (pH<3). Similar conclusions were reached in a previous conformational study of alkylated deoxynojirimycin derivatives.⁷⁴

4.1.3 Target Sulfonium Compounds

The syntheses of sulfonium salts 68 and 69 (Schemes 19 and 20) were achieved in a similar fashion to those of the ammonium salts. Thus, compound 74 was initially reacted with the D-cyclic sulfate 71b in acetone at 65° C. Slow formation of two more-polar products was observed by TLC analysis. Isolation of the mixture of products gave 88b and 89b in approximately 37% yield. On changing the solvent to 1,1,1,3,3,3-hexafluoro-2-propanol (HFIP), the yield improved to 87%. The dramatically beneficial effect of HFIP solvent on the yields for sulfonium salt formation has been noted above. The ratio of the major product 88b to the minor product 89b was 2:1. Pure samples of the two components were obtained by chromatography and characterized separately by NMR techniques.

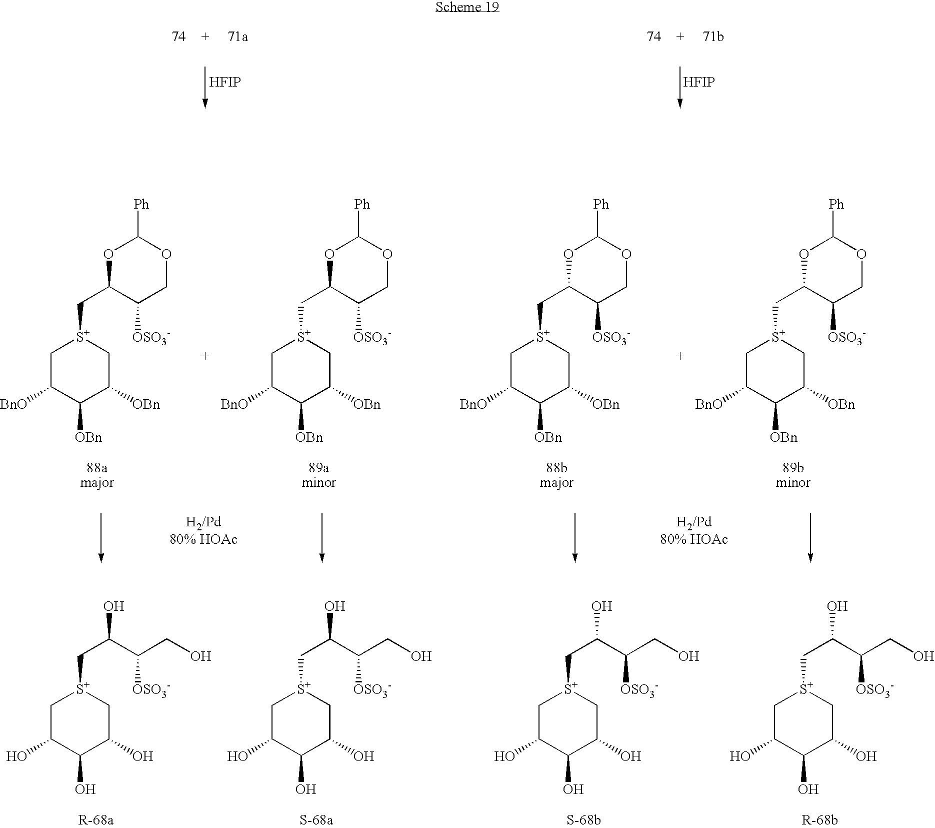

Initially, 1D ¹H NMR spectra were obtained which revealed that the two compounds were isomers, having the same number of hydrogen atoms. The similarity of the spectra of the two compounds suggested that the compounds differed in stereochemistry only at the stereogenic sulfur atom. COSY spectra permitted the assignment of the proton signals for the tetrahydrothiapyran ring and for the erythritol side chain in both compounds. Notably, it was found that all of the ring proton signals were shifted downfield relative to the parent tetrahydrothiapyran 17. This was anticipated since the positive sulfonium center is electron withdrawing. Furthermore, although it was initially expected that the three benzyloxy groups at C-2, C-3 and C-4 would favor the sterically less-hindered equatorial positions, analysis of vicinal coupling constants showed that J_{2,3} and J_{3,4}=3.5-3.9 Hz. These values are much smaller than those (J_{2,3}≈J_{3,4}≈8.9 Hz) observed for the axial-axial vicinal coupling constants in the precursor 17. Thus, the inventors reasoned that compounds 88b and 89b preferred a¹C₄ conformation, placing the three benzyloxy groups in axial positions and accounting for the small vicinal coupling constants. This conformational preference can be explained by the fact that the axial substituents at C-2 and C-4 provide stabilizing gauche electrostatic interactions of the polar benzyloxy groups with the sulfonium ion center; the group at C-3 can also provide stabilizing electrostatic interactions.²⁸ The results are reminiscent of the inventors' previous work with the sulfonium analogue of castanospermine.²⁸

The configuration at the sulfonium center was next established by means of a NOESY experiment. The NOESY spectrum for the major diastereomer showed H-1b′ correlations to H-1ax/H-1eq/H-5ax as well as H-1a′ and correlations to H-5eq/H-5ax. This isomer was thus assigned to structure 88b with the erythritol side chain occupying the equatorial orientation. The absolute configuration at sulfur was thus established as being S.

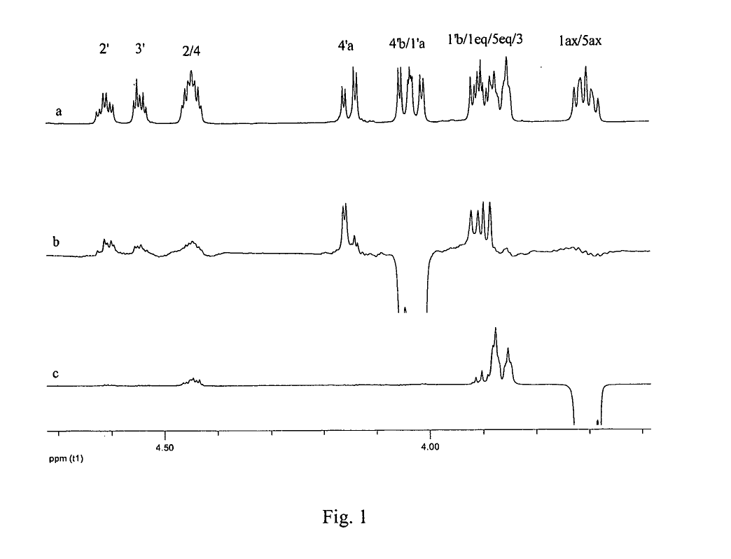

The NOESY spectrum for the minor diastereomer showed a correlation between H-1a′ and the isochronous signal assigned to H-1ax/H-1 eq, as well as a correlation between H-1b′ and H-5eq. No correlation with H-5ax was observed. This isomer was thus assigned to structure 89b, the diastereomer with the erythritol side chain in an axial orientation. The absolute configuration at sulfur was thus established as being R. Each of the diastereomers 88b and 89b was deprotected by hydrogenolysis to give sulfonium salts S-68b and R-68b, which were obtained in 81 and 95% yields, respectively. Vicinal coupling constants indicated that deprotection was accompanied in both cases by a change in the preponderant ring conformation from ¹C₄ to ⁴C₁ (S-68b J_{2,3}≈J_{3,4}≈7.2 Hz, R-68b J_{2,3}≈J_{3,4}≈9.0 Hz). Transient one-dimensional nuclear Overhauser enhancement (NOE) difference experiments confirmed that there was no configurational inversion at the sulfonium center upon removal of the benzyl and benzylidene protecting groups. Thus, the major isomer S-68b, upon irradiation of the H-4′b/H-1′a multiplet showed no NOE with the ring axial protons (FIG. 1). Irradiation of H-1ax/H-5ax showed NOEs on the H-1 eq/H-5eq/H-3 and H-2/H-4 multiplets only. No NOEs with the erythritol side chain protons were observed. These experiments provide evidence for the erythritol side chain occupying the axial position at sulfur, on the β-face and opposite to H-1ax., and confirm the S configuration at the sulfonium center for the major isomer S-68b, as was previously assigned for the protected precursor 88b.

Preferred conformations of 88b, 89b, S-68b and R-68b

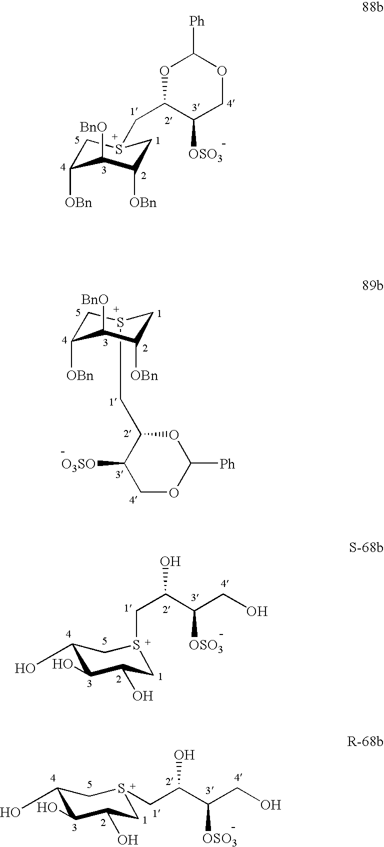

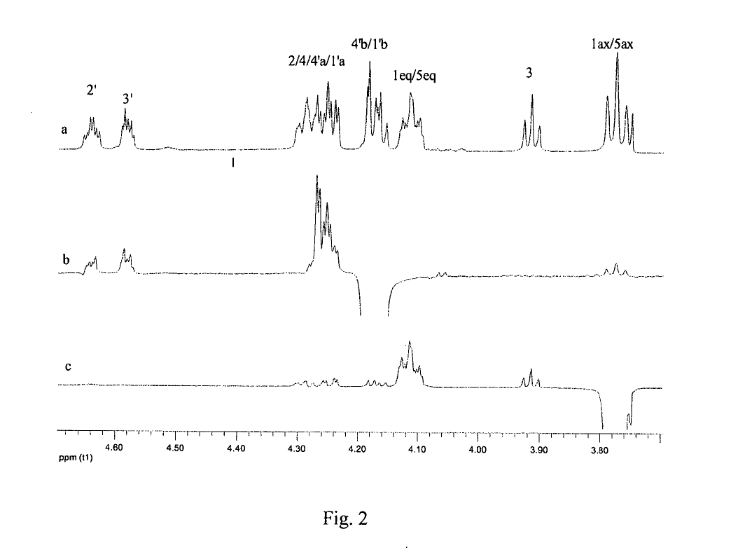

The minor isomer R-68b showed, upon irradiation of the H-4′b/H-1′b multiplet, NOE with the H-1ax/H-5ax protons (FIG. 2). Irradiation of the H-1ax/H-5ax multiplet showed NOEs with the H-4′b/H-1′b multiplet as well as to the H-2/H-4/H-4′a/H-1′a multiplet, in addition to NOEs to the ring protons. These experiments provide evidence for the erythritol side chain being present on the same face as H-1ax, occupying the α-equatorial position at sulfur, thus confirming the R configuration of the minor isomer R-68b at the sulfonium center, as was previously assigned for the protected precursor 89b.

The synthesis of the sulfonium salts from the L-cyclic sulfate 71a was examined next (Scheme 19). Compound 74 was reacted with 71a at 70° C. in HFIP solvent to give two products 88a and 89a in a 5:2 ratio (84% yield). The major diastereoisomer 88a, in which the erythritol side chain is cis to the C-3 benzyloxy group, was separated from the minor diastereoisomer 89a, with the erythritol side chain trans to the C-3 benzyloxy group. The ¹H NMR spectra were virtually identical to those of the enantiomers 88b and 89b except for small variations due to concentration, as noted above. Each of the diastereomers 88a and 89a was deprotected via hydrogenolysis to give the target compounds R-68a and S-68a.

Entry into the 5-thio-D-glucitol analogues began by treatment of 1,5-anhydro-2,3,4,6-tetra-O-benzyl-5-thio-D-glucitol 75 with the D-cyclic sulfate 71b. The reaction afforded an inseparable mixture of compounds 90b and 91b with an approximate 2:1 isomer ratio in 70% yield (Scheme 20). As in the xylitol series, the protected glucitol derivative 90b displayed an unusual ¹C₄ conformational preference, as indicated by the coupling constants. This places the three benzyloxy groups at C-2, C-3 and C-4 as well as the benzyloxymethyl group at C-5 in an axial orientation.

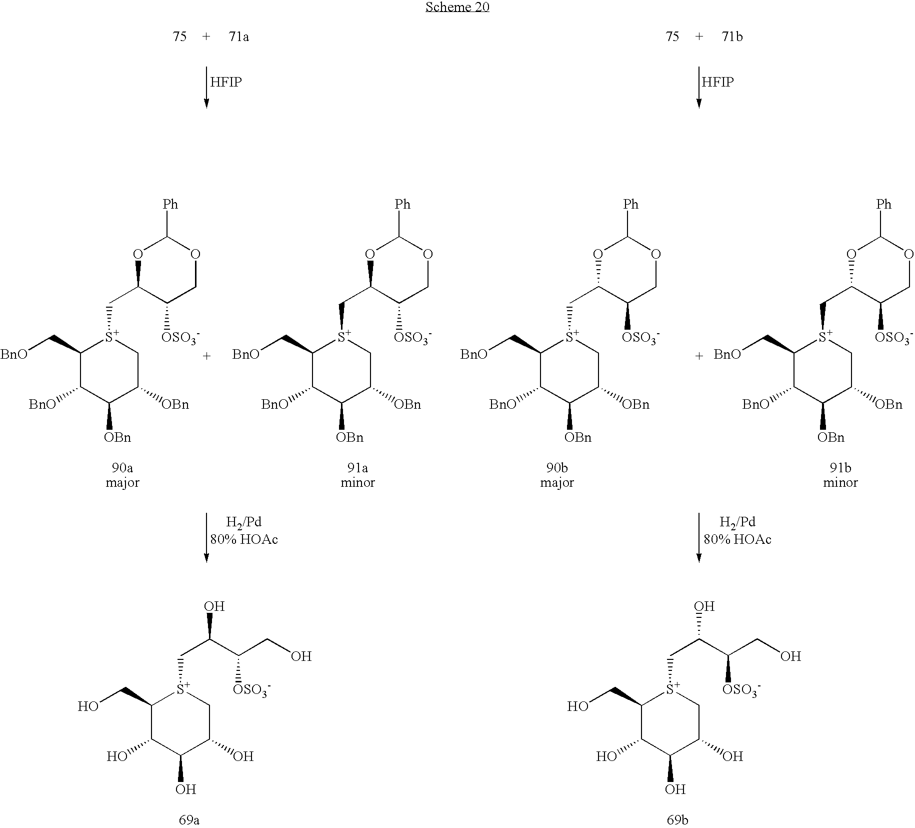

The stereochemistry at the stereogenic sulfonium center for the major isomer 90b was established by means of a NOESY experiment. A strong NOESY correlation was observed between the H-1b′ proton and the H-5 proton, thus confirming that the benzylidene-protected erythritol side chain was cis to H-5. NOEs to H-1ax and to H-6a/H-6b were not observed. Thus, the absolute configuration at the sulfonium center in the major isomer was S. Alkylation of the sulfur must occur preferentially from the α-face of 1,5-anhydro-2,3,4,6-tetra-O-benzyl-5-thio-D-glucitol 75 due to shielding of the β-face by the adjacent C-5 benzyloxymethyl group.

The mixture consisting of compounds 90b and 91b was then subjected to hydrogenolysis to give primarily 1,5-dideoxy-1,5-[[(2R, 3R)-2,4-dihydroxy-3-(sulfooxy)-butyl]-episulfoniumylidene]-D-glucitol inner salt S-69b in 81% yield (Scheme 20). Treatment of 1,5-anhydro-2,3,4,6-tetra-O-benzyl-5-thio-D-glucitol (75) with the L-cyclic sulfate 71a afforded an inseparable mixture of compounds 90a and 91a with an approximate 3:1 isomer ratio in 68% yield (Scheme 20). Whereas the achiral anhydroxylitol compound 74 generated enantiomers upon reaction with the enantiomeric D- and L-cyclic sulfates, this was not the case for the chiral compound 75. For this reaction, the products 90a and 90b are diastereomers rather than enantiomers.

The stereochemistry at the stereogenic sulfonium center for the major isomer 90a was again established by means of a NOESY experiment. A strong NOE correlation was observed between the H-1′a proton and H-5. In addition, there was also an NOE correlation between H-2′ and H-5, confirming that the benzylidene protected erythritol side chain was on the same side as H-5. NOEs to H-1ax and to H-6a/H-6b were not observed. Thus, the absolute configuration at the sulfonium center for compound 90a was R; that is, the same stereochemistry at sulfur previously found for the diastereoisomer 90b. (Note: The change in R/S configuration between 90a and 90b due to sequence rules does not imply a change in stereochemistry at sulfur in this case). Therefore, independent of the configuration (71a or 71b) of the cyclic sulfate reagent, in both cases, alkylation at sulfur occurred preferentially from the least hindered β-face of compound 75.

The mixture containing 90a and 91a was then subjected to hydrogenolysis to give primarily 1,5-dideoxy-1,5-[[(2R,3R)-2,4-dihydroxy-3-(sulfooxy)-butyl]-R-episulfonium-ylidene]-D-glucitol inner salt R-69a in 67% yield (Scheme 20).

Upon removal of the protecting groups, compounds R-69a and S-69b adopted a ⁴C₁ conformation, as indicated by the vicinal proton coupling constants. This places all of the ring substituents in an equatorial orientation, as observed for the xylitol series.

4.1.4 Target Selenonium Compounds

The tetrahydroselenapyran 76 was coupled to the D-cyclic sulfate 71b in HFIP solvent and afforded an inseparable mixture of two compounds, 92b and 93b in a 1:4 ratio in 96% yield (Scheme 21). These two compounds are diastereoisomers at the stereogenic selenium center. Alkylation can occur on selenium to give, as with sulfur, the benzylidene protected erythritol side chain either cis to the C-3 benzyloxy group or trans to the C-3 benzyloxy group. It was found by comparison of the NMR data to those of the sulfonium analogues 88b/89b, and by analysis of the NOESY spectrum (see below), that the major product, 93b, was that in which the benzylidene-protected erythritol side chain was trans to the benzyloxy group at C-3. The minor product, 92b, was that in which the benzylidene protected erythritol side chain was cis to the C-3 benzyloxy group. Curiously, the ratio was opposite to the results obtained with the tetrahydrothiapyran products 88b and 89b for which the major isomer was the cis isomer. The predominant conformations observed in both compounds 92b and 93b were, as with the corresponding thio analogues, those which placed all three benzyloxy groups in an axial arrangement, thus favoring ¹C₄ conformations, as evidenced by the coupling constants. The major isomer 93b in its preferred ¹C₄ conformation places the selenonium alkyl group in the axial position. The longer C—Se bonds in compounds 92b/93b compared to the thio analogues must result in less severe gauche steric interactions between the selenonium alkyl group and C-2 and C-4.

The mixture consisting of compounds 92b and 93b was then deprotected via hydrogenolysis to give mostly one diastereoisomer of 70b, in 39% yield (Scheme 21). The low yield was due to catalyst poisoning by decomposition products and the reaction could not be brought to completion. This major compound was characterized by NMR techniques and found to be 1,5-dideoxy-1,5-[[(2R,3R)-2,4-dihydroxy-3-(sulfooxy)-butyl]-R-episelenoniumylidene]-xylitol inner salt R-70b.

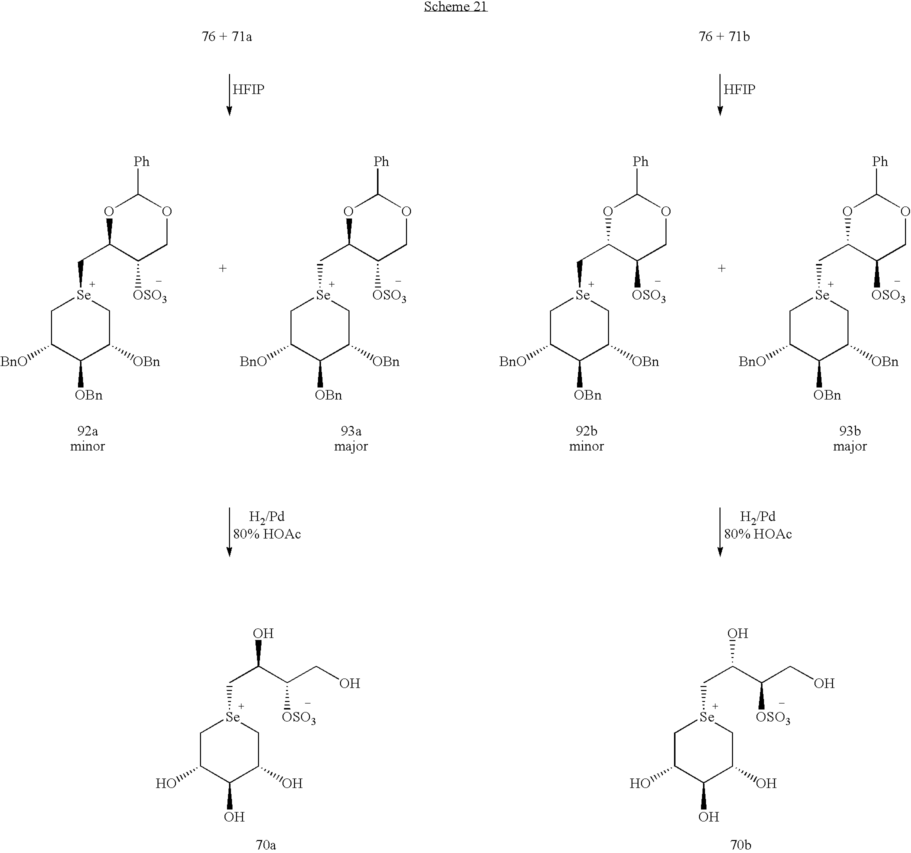

Reaction of the selenoether 76 with the L-cyclic sulfate 71a was also performed. The product was an inseparable mixture of two diastereoisomers at the stereogenic selenium center, 92a and 93a, in a 1:3 ratio. (Scheme 21).

The configuration at the stereogenic selenonium centers for the enantiomers 93a and 93b was confirmed by means of NOESY experiments performed on the mixtures of the compounds containing their minor diastereomers. The major isomer in each case was found to be that in which the erythritol side chain occupied the axial position in the preferred ¹C₄ conformation. This was evidenced by correlations between H-1b′ and H-5eq as well as correlations between H-1′a and H-1eq. An axial preference would imply correlations between H-1′a/H-1′b and H-5eq, and H-1′a/H-1′b and H-1eq only, since free rotation about the C-1′—Se bond would not permit the H-1′a and H-1′b protons to interact with the axial C-1 and C-5 protons as these are on the opposite side of the selenoether ring. Therefore, NOEs would not be expected between H-1′a/H-1′b and H-1ax/H-1eq. On the other hand, an equatorial preference would imply correlations between H-1′a/H-1′b to H-1ax and H-5ax as well as possibly to H-1eq and H-5eq. Thus, for compound 93b the absolute configuration at the selenium center is R and that for the enantiomeric 93a is S. In both cases, the erythritol side chain is cis to the benzyloxy groups at C-2 and C-4 and trans to the C-3 benzyloxy group.

The mixture consisting of 92a and 93a was then deprotected by hydrogenolysis to afford mostly one diastereoisomer of 70a in 25% yield (Scheme 21). The major compound was characterized by NMR techniques and found to be the desired 1,5-dideoxy-1,5-[[(2S,3S)-2,4-dihydroxy-3-(sulfooxy)-butyl]-S-episelenoniumylidene]-xylitol inner salt S-70a, the enantiomer of compound R-70b.

4.2 Chain Extended Homologues of Salacinol

The synthesis of Salacinol and some of its enantio-and diastereoisomers is described above. In addition, the inventors have developed a strategy for synthesizing Salacinol homologues having an extended alditol side chain. In one embodiment, the side chain may have 5 or 6 carbons. Four Salcinol homologues 94-97 are shown below.

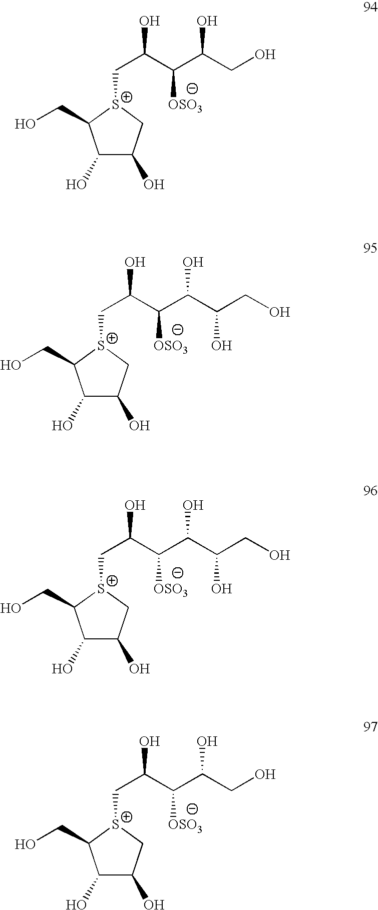

In principle, the desired compounds could be obtained from the sulfonium-sulfate disaccharide analogues 98-101; such analogues are representatives of a new class of carbohydrate derivatives and may have interesting properties in and of themselves. They are disaccharide analogues in which a permanent positive charge resides on the non-reducing ring and linkage heteroatom simultaneously. As such, they may be mimics of the partial positive charge that is generated on analogous atoms at the transition state stage of enzyme catalyzed glycoside hydrolysis.

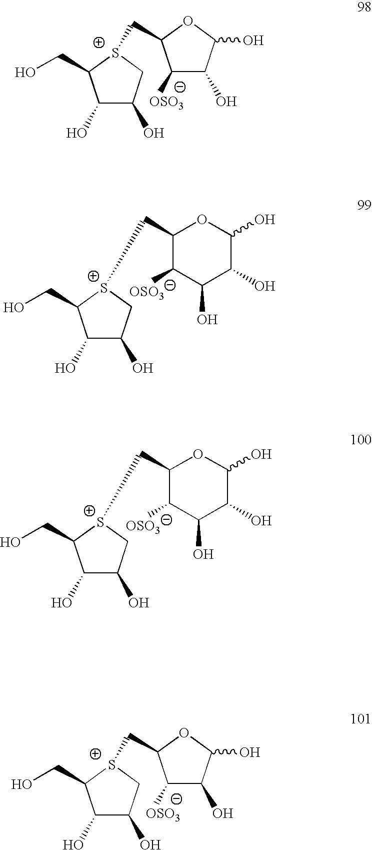

The inventors' synthetic strategy was similar to that used to the inventors' advantage for related structures as described above. This involves opening of a 1,3-cyclic sulfate ring by nucleophilic attack of a sulfide. In this case the target structures were chosen partly due to the availability of appropriate cyclic sulfate derivatives. A literature survey, searching for 1,3-cyclic sulfates of carbohydrate derivatives, returned the glucopyranoside 4,6-O-cyclic sulfates 102³⁷ and 103³⁸ as well as the xylose derivative 104³⁹ and the galactose derivative 105.⁴⁰

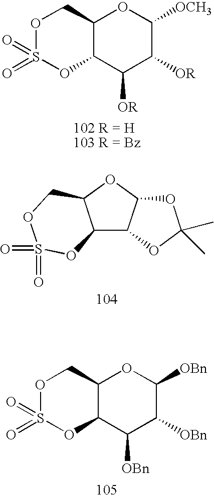

These derivatives have been shown to react with oxygen, nitrogen or sulfur nucleophiles selectively at the primary carbon. The methyl pyranosides 102 and 103 were rejected due to the probable harsh conditions necessary for hydrolysis of the glycoside bond during the deprotection of the proposed, and possibly sensitive, sulfonium intermediates. Compounds 104 and 105 were deemed to be more suitable and could be prepared by the literature methods. Three other cyclic sulfates could be prepared by new methods. Benzyl glucopyranoside 4,6-cyclic sulfate 107 could be prepared by the Sharpless method⁸¹ from known benzyl glucopyranoside 106⁴¹ and similar treatment of the methyl or benzyl arabinofuranosides 108⁴² and 109 would yield cyclic sulfates 110 and 111 (Scheme 22).

Schemes for the synthesis of the target chain-extended homologue compounds are shown in the schemes that follow and the testing of the general strategy is described for the reaction of the cyclic sulfate 105.

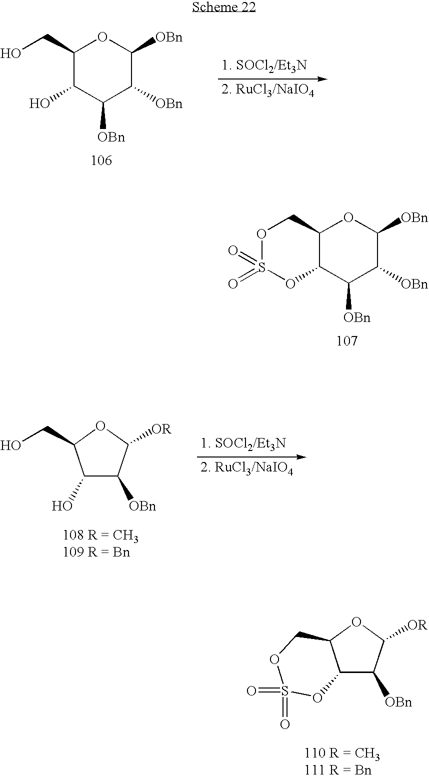

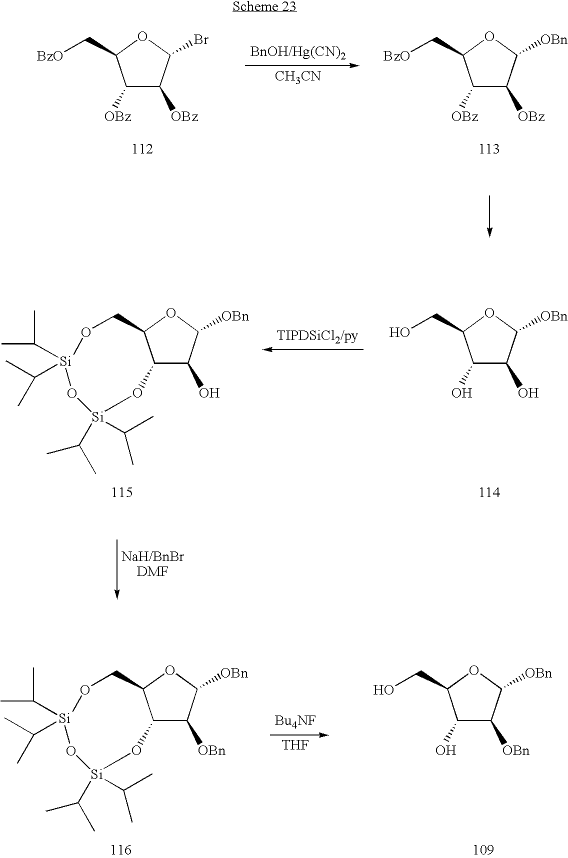

Sulfide 117 was available from earlier work²⁵ and could be prepared more conveniently by a method analogous to that developed for the corresponding selenium derivative.²⁶ Compound 117 was reacted with cyclic sulfate 105 to give protected sulfonium sulfate compound 119 (Scheme 24). The solvent was the unusual solvent 1,1,1,3,3,3-hexafluoroisopropanol (HFIP) which the inventors have found to offer significant advantages in reactions to form sulfonium salts as mentioned above. Compound 119 was deprotected by hydrogenolysis with H₂ over a Pd catalyst to give hemiacetal derivative 99 as a mixture of anomeors. Reduction of the mixture with sodium borohydride yielded compound 95, a chain extended analogue of Salacinol.

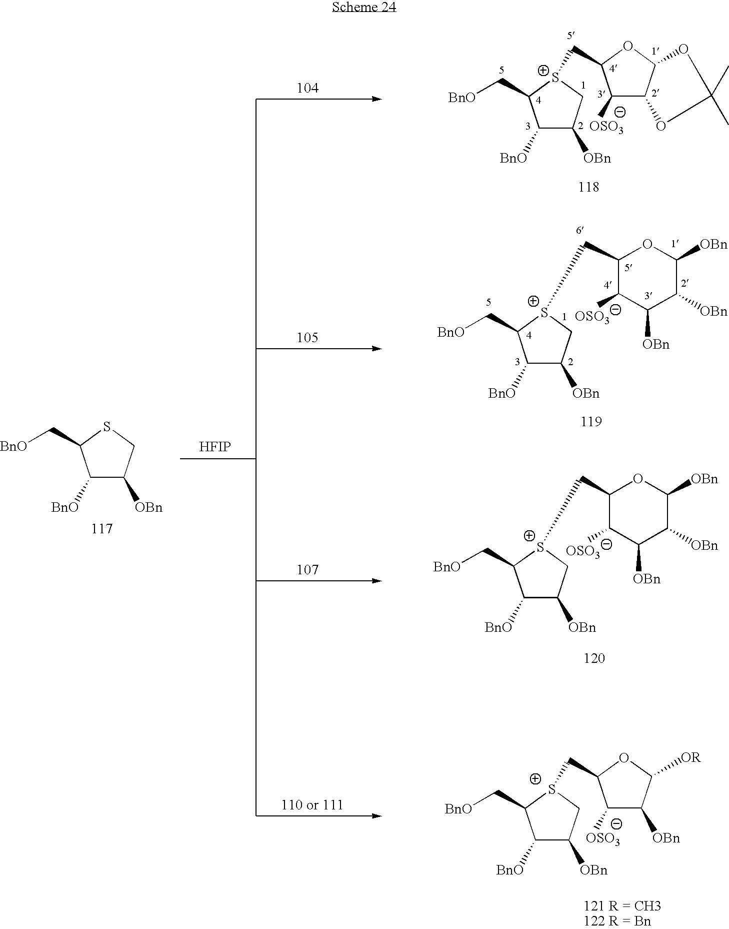

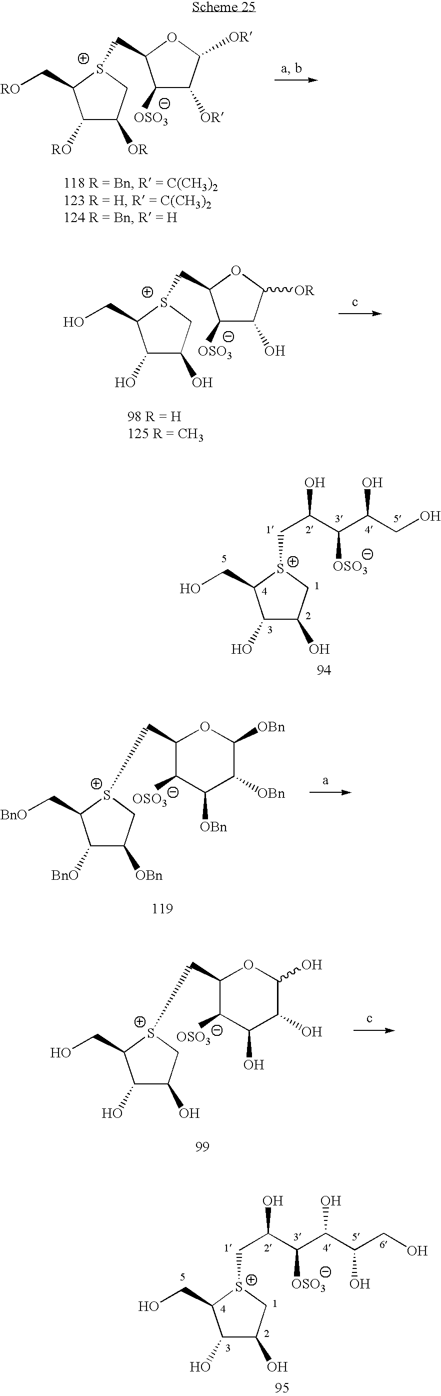

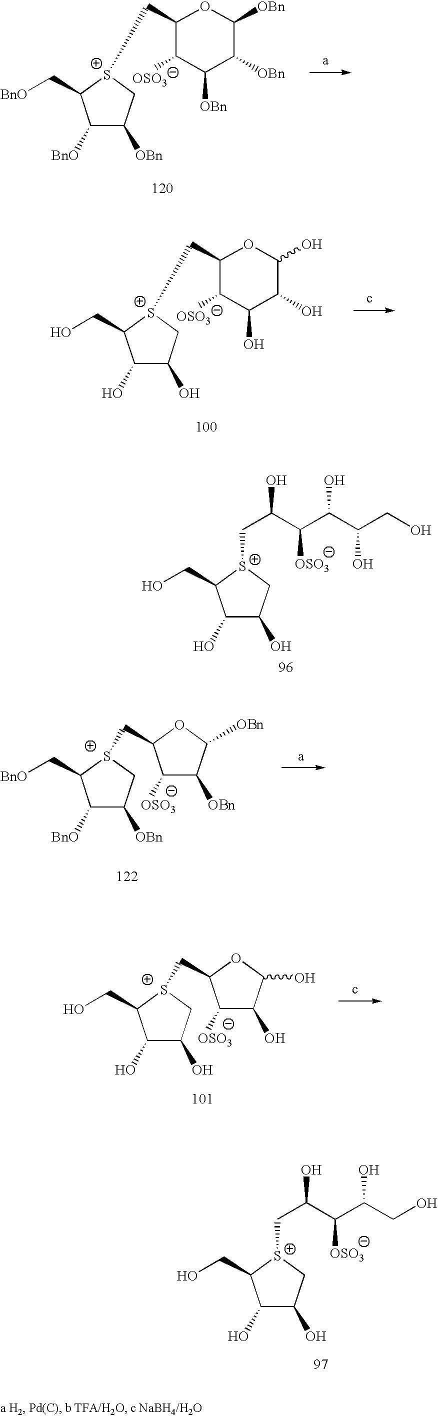

### 5.0 EXAMPLES

The following examples will further illustrate the invention in greater detail although it will be appreciated that the invention is not limited to the specific examples.

5.1 Experimental Methods

Optical rotations were measured at 20° C. ¹H and ¹³C NMR spectra were recorded at 400.13 and 100.6 MHz for proton and carbon respectively. All assignments were confirmed with the aid of two-dimensional ¹H,¹H(COSYDFTP) or ¹H,¹³C (INVBTP) experiments using standard Bruker pulse programs. MALDI-TOF mass spectra were obtained for samples dispersed in a 2,5-dihydroxybenzoic acid matrix using a Perseptive Biosystems Voyager-DE instrument. Silica gel for chromatography was Merck kieselgel 60. High resolution mass spectra were LSIMS (Fab), run on a Kratos Concept H double focussing mass spectrometer at 10000 RP.

5.2 Preparation of Intermediates

### 5.2.1 Example 1

### Preparation of Cyclic Sulfate (7) (Scheme 2)

### Step 1-2,4-O-Benzylidene-D-erythritol (5)

Compound (5) was prepared from 4,6-O-benzylidene-D-glucose (4) according to standard procedures.^{9,10} Compound (5) has been mentioned by MacDonald et al.,¹⁰ without characterization, which is therefore dealt with here. Mp 138-139° C.; [α]_{D}-44° (c 1.0, MeOH); ¹H NMR (CD₃OD): δ 7.53-7.28 (5H, m, Ar), 5.53 (1H, s, H-5), 4.2 (1H, dd, J=10.1, 3.6 Hz, H-4a), 3.92 (1H, dd, J=12.1, 1.7 Hz, H-1a), 3.74 (1H, dd, J=12.1, 5.7 Hz, H-1b), 3.67-3.55 (3H, m, H-3, H-2, H-4b); ¹³C NMR (100.6 MHz, CD₃OD): δ 139.52 (Cᵢₚₛₒ), 129.77 (Cₚₐᵣₐ), 128.99, 127.49 (4Cₒᵣₜₕₒ₊ₘₑₜₐ), 102.36 (C-5), 84.22 (C-3), 72.21 (C-4), 62.76 (C-1), 62.59 (C-2); MALDI-TOF MS: m/e 211 (M⁺+H), 233 (M⁺+Na). Anal. Calcd for C₁₁H₁₄O₄: C, 62.83; H, 6.72. Found: C, 62.96; H, 6.55.

### Step 2-2,4-O-Benzylidene-D-erythritol-1,3-cyclic Sulfite (6)

A solution of the diol (5) (4.5 g, 21 mmol) and Et₃N (11 mL, 4equiv) in dry CH₂Cl₂ (90 mL) was added dropwise to a solution of SOCl₂ (2.4 mL, 1.5equiv) in dry CH₂Cl₂ (60 mL), with stirring in an ice-bath under an N₂ atmosphere. Stirring was continued at 0° C., until TLC (hex:EtOAc, 4:1) showed complete disappearance of the starting material. The mixture was diluted with CH₂Cl₂ (150 mL) and washed with H₂O (150 mL) and brine (150 mL). The organic solution was dried (Na₂SO₄) and concentrated on a rotary evaporator. The product was purified by flash chromatography [hex:EtOAc, 4:1+0.1% Et₃N] to give a mixture of two diastereomers (4.5 g, 82%). One of the isomers was selectively recrystallized from EtOAc:hex. Mp 137-139° C.; [α]_{D}+32° (c 1.0, CH₂Cl₂); ¹H NMR (CD₂Cl₂): δ 7.48-7.36 (5H, m, Ar), 5.68 (1H, s, H-5), 5.04 (1H, ddd, J=10.4, 9.5, 5.0 Hz, H-3), 4.80 (1H, dd, J=10.4, 10.4 Hz, H-1a), 4.24 (1H, dd, J=10.5, 5.0 Hz, H-4e), 4.18 (1H, ddd, J=10.4, 9.5, 4.8 Hz, H-2), 4.06 (1H, dd, J=10.4, 4.8 Hz, H-1e), 3.89(1H, dd, J=10.5, 10.4 Hz, H-4a); ¹³C NMR (100.6 MHz, CD₂Cl₂): δ 137.14 (Cᵢₚₛₒ), 129.74 (Cₚₐᵣₐ), 128.65, 126.50 (4Cₒᵣₜₕₒ₊ₘₑₜₐ), 102.72 (C-5), 73.56 (C-2), 68.16 (C-4), 63.90 (C-3), 60.18 (C-1). Anal. Calcd for C₁₁H₁₂O₅S: C, 51.55; H, 4.72. Found: C, 51.80; H, 4.66.

### Step 3-2,4-O-Benzylidene-D-erythritol-1,3-cyclic Sulfate (7)

The cyclic sulfite (6) (3.5 g, 14 mmol) was dissolved in a mixture of MeCN (50 mL) and CCl₄ (50 mL), and NaIO₄ (4.1 g, 1.5equiv) and RuCl₃.H₂O (50 mg) were added followed by H₂O (50 mL). The mixture was stirred vigorously at rt until TLC (hex:EtOAc,4:1) showed complete disappearance of the starting material. The mixture was diluted with Et₂O (200 mL) and washed with H₂O (200 mL) and brine (200 mL). The organic solution was dried (Na₂SO₄) and concentrated on a rotary evaporator. The product was purified by flash chromatography [hex:EtOAc, 4:1+0.1% Et₃N] to yield a white solid (3.5 g, 95%). A portion of the product was recrystallized from EtOAc:hex. Mp 115-125° C. (dec); [α]_{D}+4° (c 1.0, CHCl₃); ¹H NMR (CD₂Cl₂): δ 7.48-7.37 (5H, m, Ar), 5.65 (1H, s, H-5), 4.86 (1H, ddd, J=10.2, 9.8, 5.0 Hz, H-3), 4.76 (1H, dd, 0.1=10.7, 10.5 Hz, H-1a), 4.65 (1H, dd, J=10.5, 5.0 Hz, H-1e), 4.44 (1H, dd,J=10.5, 5.0 Hz, H-4e), 4.25 (1H, ddd, J=10.7, 9.8, 5.0 Hz, H-2), 3.97 (1H, dd, J=10.5, 10.2 Hz, H-4a); ¹³C NMR (100.6 MHz, CD₂Cl₂): δ 136.32 (Cᵢₚₛₒ), 130.03 (Cₚₐᵣₐ), 128.74, 126.52 (4Cₒᵣₜₕₒ₊ₘₑₜₐ), 102.98 (C-5), 75.74 (C-3), 73.19 (C-1), 71.68 (C-2), 67.64 (C-4); MALDI-TOF MS: m/e 273 (M⁺+H), Anal. Calcd for C₁₁H₁₂O₆S: C, 48.52; H, 4.45. Found: C, 48.43; H, 4.39.

### 5.2.2 Example 2

### Preparation of Thio-Arabinitol (Scheme 4)

### 1,4-Anhydro-2,3,5-tri-O-benzyl-4-thio-D-arabinitol(12)

A mixture of 1,4-anhydro-3-O-benzyl-4-thio-D-arabinitol (1.0 g, 4.2 mmol) and 60% NaH (0.85 g, 5equiv) in DMF (20 mL) was stirred in an ice-bath for 1 h. A solution of benzyl bromide (1.9 mL, 3.8equiv) in DMF (5 mL) was added and the solution was stirred at rt for 3 h. The mixture was added to ice-water (150 mL) and extracted with Et₂O (150 mL). The organic solution was dried (Na₂SO₄) and concentrated. The product was purified by flash chromatography [hex:EtOAc, 4:1] to give a syrup (1.6 g, 90%). [α]_{D}+5° (c 1.6, CHCl₃); ¹H NMR (CDCl₃): δ 7.38-7.23 (15H, m, Ar), 4.64-4.45 (6H, m, CH₂Ph), 4.19 (1H, dd, J=8.9, 4.6 Hz, H-2), 4.11 (1H, dd, J=7.2, 3.8 Hz, H-3), 3.69 (1H, dd, J=8.8, 7.6 Hz, H-5a), 3.57 (1H, ddd, J=7.5, 6.4, 3.6 Hz, H-4), 3.50 (1H, dd, J=8.9, 6.3 Hz, H-5b), 3.08 (1H, dd, J=11.4, 5.1 Hz, H-1a), 2.91 (1H, dd, J=11.4, 4.6 Hz, H-1b). ¹³C NMR (100.6 MHz, CDCl₃): δ 138.16, 138.06, 137.88 (3Cᵢₚₛₒ), 128.40-127.59 (15C_{Ar}), 85.08 (C-3), 85.04 (C-2), 73.01 (CH₂Ph), 72.34 (C-5), 71.85, 71.50(2CH₂Ph), 48.99 (C-4), 33.10 (C-1). Anal. Calcd for C₂₆H₂₈O₃S: C, 74.25; H, 6.72. Found: C, 74.18; H, 6.53.

### 5.2.3 Example 3

### Preparation of Seleno-Arabinitol (Scheme 6)

### 1,4-Anhydro-2,3,5-tri-O-benzyl-4-seleno-D-arabinitol (20)

Selenium metal (1.1 g, 14 mmol) was added to liquid NH₃ (60 mL) in a −50° C. bath and small pieces of Na (0.71 g) were added until a blue color appeared. A small portion of selenium (20 mg) was added to remove the blue color. NH₃ was removed by warming on a water bath and DMF was added and removed under high vacuum to remove the rest of NH₃. A solution of the mesylated compound (18) (7.4 g, 12.7 mmol) in DMF (100 mL) was added and the mixture was stirred under N₂ in a 70° C. bath for 3 h. The mixture was cooled and the solvent was removed on high vacuum. The product was partitioned between CH₂Cl₂ (150 mL) and water (50 mL), and the organic solution was washed with water (50 mL) and brine (50 mL) and dried (MgSO₄). The product was purified by flash chromatography (hex:EtOAc, 3:1) to give a yellow oil (4.74 g, 80%). [α]_{D}+22° (c 1.3, CHCl₃); ¹H NMR (CDCl₃): δ 7.22-7.48 (15H, m, Ar), 4.67, 4.61 (2H, 2d, J=11.8 Hz, CH₂Ph), 4.56, 4.48 (2H, 2d, J=12.1 Hz,CH₂Ph), 4.53, 4.50 (2H, 2d, CH₂Ph), 4.22 (1H, dd, J=10.1, 5.1 Hz, H-2), 4.07 (1H, dd, J=4.6, 4.6 Hz, H-3), 3.85 (1H, dd, J=9.2, 7.6 Hz, H-5a), 3.77 (1H, ddd, J=7.5, 6.9, 4.5 Hz, H-4), 3.53 (1H, dd, J=9.1, 6.8 Hz, H-5b), 3.11 (1H, dd, J=10.4, 5.1 Hz, H-1a), 2.96 (1H, dd, J=10.4, 5.3 Hz, H-1b). ¹³C NMR (100.6 MHz, CDCl₃): δ 138.24, 138.21, 138.06 (3Cᵢₚₛₒ), 128.40-127.60 (15C_{Ar}), 85.93 (C-2), 85.63 (C-3), 72.96 (C-5, CH₂Ph), 72.14, 71.50(2CH₂Ph), 42.59 (C-4), 23.96 (C-1). Anal. Calcd for C₂₆H₂₈O₃Se: C, 66.65; H, 6.03. Found: C, 66.49; H, 6.05.

### 5.2.4 Example 4

### General Procedure for the Synthesis of the Protected Sulfonium, Selenonium and Ammonium Sulfates (21), (22), (24), (26), (27) (28). (30), (31) (Schemes 7-14)

The thio, aza or selenosugar (3 mmol) and the cyclic sulfate (1.2equiv) were dissolved in dry acetone (in the case of (21), (22), (24), (26), (27) and (28)) or dry methanol (in the case of (30) and (31)) (0.5 mL) and anhydrous K₂CO₃ (7 mg) was added. The mixture was stirred in a Caries tube in an oil-bath (75° C.) overnight. The solvent was removed under reduced pressure and the product was purified by column chromatography.

### 1-((1′,4′-Anhydro-2′,3′,5′-tri-O-benzyl-4′-thio-D-arabinitol)-4′-S-yl)-2,4-O-benzylidene-1-deoxy-L-erythritol-3-sulfate (21)

Column chromatography [CHCl₃:MeOH, 10:1+0.1% Et₃N] of the crude product gave an amorphous solid (33%). [α]_{D}−11.9° (c 1.7, CH₂Cl₂); ¹H NMR (CD₂Cl₂): δ 7.49-7.12 (20H, m, Ar), 5.54 (1H, s, H-5), 4.59 (1H, ddd, J=9.9, 5.4, 4.5 Hz, H-3), 4.55-4.33 (8H, m, 4CH₂Ph, H-2′, H-4a, H-1a, H-3′), 4.29 (1H, dt, J=9.5, 3.0 Hz, H-2), 4.25 and 4.15 (2H, 2d, J=11.9 Hz, CH₂Ph), 4.04 (1H, m, H-1′a) 4.02-3.95 (2H, m, H-4′, H-1b), 3.78 (1H, dd, J=10.7, 10.7 Hz, H-4b), 3.74 (1H, dd, J=13.6, 3.8 Hz, H-1′b), 3.62 (1H, dd, J=9.9, 8.6 Hz, H-5′a), 3.54 (1H, dd, J=9.9, 7.2 Hz, H-5′b); ¹³C NMR (100.6 MHz, CD₂Cl₂): δ 137.34, 137.24, 136.56, 136.39 (4Cᵢₚₛₒ), 129.73-126.62 (20C_{Ar}), 101.95 (C-5), 83.75 (C-3′), 82.82 (C-2′), 76.80 (C-2), 73.73, 72.84, 72.52 (3CH₂Ph), 69.54.(C-4), 67.01 (C-5′), 66.48 (C-3), 65.27 (C-4′), 49.67 (C-1), 48.28 (C-1′); MALDI-TOF MS: m/e 693 (M⁺+H). Anal. Calcd for C₃₇H₄₀O₉S₂: C, 64.14; H, 5.82. Found: C, 63.88; H, 5.83.

### 1-((1′,4′-Anhydro-2′,3′,5′-tri-O-benzyl-4′-thio-D-arabinitol)-4′-S-yl)-2,4-O-benzylidene-1-deoxy-D-erythritol-3-sulfate (22)

Column chromatography [CHCl₃:MeOH, 10:1+0.1% Et₃N] of the crude product gave an amorphous solid (79%). [α]_{D}−46.9° (c 0.65, CH₂Cl₂); ¹H NMR (CD₂Cl₂): δ 7.43-7.10 (20H, m, Ar), 5.49 (1H, s, H-5), 4.62-4.34 (11H, m, CH₂Ph, H-3, H-4a, H-2′, H-1a, H-3′), 4.30-4.21 (2H, m, H-2, H-4′), 3.96 (1H, dd, J=9.7, 6.2 Hz, H-5′a), 3.90 (1H, dd, J=13.3, 3.4 Hz, H-1b), 3.82 (1H, dd, J=9.8, 9.8 Hz, H-5′b), 3.79-3.71 (2H, m, H-1′a, H-4b), 3.51 (1H, dd, J=13.2, 3.9 Hz, H-1′b); ¹³C NMR (100.6 MHz, CD₂Cl₂): δ 137.62, 137.27, 136.48, 136.29 (4Cᵢₚₛₒ), 129.80-126.56 (20C_{Ar}), 102.16 (C-5), 84.25 (C-3′), 82.56 (C-2′), 77.07 (C-2), 74.02, 72.74 (3CH₂Ph), 69.75 (C-4), 67.19 (C-5′), 66.82 (C-3), 65.76 (C-4′), 50.41 (C-1), 49.60 (C-1′); MALDI-TOF MS: m/e 693 (M⁺+H). Anal. Calcd for C₃₇H₄₀O₉S₂: C, 64.14; H, 5.82. Found: C, 64.16; H, 5.73.

### 1-((1′,4′-Anhydro-2′,3′,5′-tri-O-benzyl-4′-thio-L-arabinitol)-4′-S-yl)-2,4-O-benzylidene-1-deoxy-D-erythritol-3-sulfate (24)

Column chromatography [CHCl₃:MeOH, 10:1+0.1% Et₃N] of the crude product gave an amorphous solid (40%). [α]_{D}+14.3° (c 1.4, CH₂Cl₂); ¹H NMR (CD₂Cl₂): δ 7.49-7.12 (20H, m, Ar), 5.55 (1H, s, H-5), 4.60 (1H, ddd, J=9.8, 5.5, 4.5 Hz, H-3), 4.55-4.44 (5H, m, 3CH₂Ph, H-2′, H-4a), 4.42 (1H, dd, J=13.3, 2.3 Hz, H-1a), 4.39-4.34 (2H, m, CH₂Ph, H-3′), 4.28 (1H, dt, J=9.8, 2.9 Hz, H-2), 4.24 and 4.14 (2H, 2d, J=11.9 Hz, CH₂Ph), 4.10 (1H, d, J=13.4 Hz H-1′a), 3.98-3.90 (2H, m, H-4′, H-1b), 3.78 (1H, dd, J=10.5, 10.5 Hz, H-4b), 3.67 (1H, dd, J=13.4, 3.8 Hz, H-1′b), 3.62 (1H, dd, J=9.9, 8.7 Hz, H-5′a), 3.53 (1H, dd, J=9.9, 7.2 Hz, H-5′b); ¹³C NMR (100.6 MHz, CD₂Cl₂): δ 137.32, 137.26, 136.48, 136.25 (4Cᵢₚₛₒ), 129.79-126.64 (20C_{Ar}), 102.06 (C-5), 83.96 (C-3′), 82.74 (C-2′), 76.93 (C-2), 73.81, 72.97, 72.57 (3CH₂Ph), 69.59.(C-4), 67.07 (C-5′), 66.36 (C-3), 66.31 (C-4′), 49.96 (C-1), 48.52 (C-1′). Anal. Calcd for C₃₇H₄₀O₉S₂: C, 64.14; H, 5.82. Found: C, 64.13; H, 5.74.

### 1-((1′,4′-Anhydro-3′-O-benzyl-4′-thio-D-arabinitol)-4′-S-yl)-2,4-O-benzylidene-1-deoxy-L-erythritol-3-sulfate (26)

Column chromatography [CHCl₃:MeOH, 10:1+0.1% Et₃N] of the crude product gave an amorphous solid (32%).; ¹H NMR (CD₂Cl₂): δ 7.49-7.26 (10H, m, Ar), 6.22 (1H, d, J=4.4 Hz, 2′-OH), 5.54 (1H, s, H-5), 4.96 (1H, br-s, H-2′), 4.64 (1H, d, J=11.6 Hz, CH₂Ph), 4.64-4.62 (1H, m, 5′-OH), 4.56 (1H, d, J=11.6 Hz, CH₂Ph), 4.54-4.48 (1H, m, H-3), 4.46 (1H, dd, J=10.5, 5.4 Hz, H-4a), 4.33-4.25 (3H, m, H-3′, H-2, H-1′a), 4.12 (1H, dd, J=13.5, 2.6 Hz, H-1a), 4.12-4.09 (1H, m, H-4′), 4.01 (1H, dd, J=13.5, 3.4 Hz, H-1b), 3.92-3.82 (2H, m, H-5′a, H-5′b), 3.78 (1H, dd, J=10.5, 10.1 Hz, H-4b), 3.67 (1H, dd, J=13.5, 3.9 Hz, H-1′b); ¹³C NMR (100.6 MHz, CD₂Cl₂): δ 136.92, 136.73 (2Cᵢₚₛₒ), 129.97-126.61 (10C_{Ar}), 102.32 (C-5), 88.45 (C-3′), 76.61 (C-2), 76.22 (C-2′), 72.96 (CH₂Ph), 71.24 (C-4′), 69.27 (C-4), 66.96 (C-3), 60.51 (C-5′), 52.43 (C-1′), 48.30 (C-1); MALDI-TOF MS: m/e 513 (M⁺+H). Anal. Calcd for C₂₃H₂₈O₉S₂: C, 53.89; H, 5.51. Found: C, 53.64; H, 5.34.

### 1-((1′,4′-Anhydro-2′,3′,5′-tri-O-benzyl-4′-seleno-D-arabinitol)-4′-Se-yl)-2,4-O-benzylidene-1-deoxy-L-erythritol-3-sulfate (27)

Column chromatography [CHCl₃:MeOH, 15:1] of the crude product gave an amorphous solid (86%). NMR showed the presence of two isomers (7:1) at the stereogenic selenium center which were separated on analytical HPLC [acetonitrile/H₂O]. Anal. Calcd for C₃₇H₄₀O₉SSe: C, 59.99; H, 5.45. Found: C, 59.91; H, 5.44.

### 1-((1′,4′-Anhydro-2′,3′,5′-tri-O-benzyl-4′-seleno-D-arabinitol)-4′-Se-yl)-2,4-O-benzylidene-1-deoxy-D-erythritol-3-sulfate (28)

Column chromatography [CHCl₃:MeOH, 15:1] of the crude product gave an amorphous solid (96%). NMR showed the presence of two isomers (3:1) at the stereogenic selenium center which were separated on analytical HPLC [acetonitrile/H₂O]. Anal. Calcd for C₃₇H₄₀O₉SSe: C, 59.99; H, 5.45. Found: C, 59.91; H, 5.37.

### 1-((1′,4′-Dideoxy-1′,4′-imino-D-arabinitol)-4′-N-yl)-2,4-O-benzylidene-1-deoxy-L-erythritol-3-sulfate (30)

A mixture of 1,4-Dideoxy-1,4-imino-D-arabinitol (19) (100 mg, 0.7 mmol) and 2,4-O-benzylidene-L-erythritol-1,3-cyclic sulfate (10) (235 mg, 1.2equiv) were dissolved in dry MeOH (0.5 mL) and anhydrous K₂CO₃ (15 mg) was added. The mixture was stirred in a Caries tube in an oil-bath (75° C.) overnight. The solvent was removed under reduced pressure and column chromatography [CH₂Cl₂:MeOH, 4.5:1] of the crude product gave an amorphous solid (219 mg, 72%). ¹H NMR (CD₃OD): δ 7.53-7.30 (5H, m, Ar), 5.61 (1H, s, H-5), 4.53 (1H, dd, J=11.1, 5.2 Hz, H-4a), 4.25 (1H, m, H-2), 4.20 (1H, ddd, J=9.8, 5.2, 4.4 Hz, H-3), 4.11 (1H, br-s, H-2′), 3.99-3.84 (4H, m, H-1a, H-3′, H-5′a, H-5′b), 3.82 (1H, dd, J=10.7, 9.8 Hz H-4b) 3.58 (1H, m, H-1′a), 3.55-3.42 (2H, m, H-1′b, H-4′), 3.38 (1H, m, H-1b); ¹³C NMR (100.6 MHz, CD₃OD): δ 138.72 (Cᵢₚₛₒ), 130.12 (Cₚₐᵣₐ), 129.21, 127.39 (4Cₒᵣₜₕₒ₊ₘₑₜₐ), 102.33 (C-5), 78.01 (C-4′, C-3′, C-2), 76.31 (C-2′), 70.29 (C-4), 69.02 (C-3), 62.64 (C-1′), 60.51 (C-5′), 58.46 (C-1); MALDI-TOF MS: m/e 428 (M⁺+Na), 406 (M⁺+H); HRMS. Calcd for C₁₆H₂₃O₉SN (M+H): 406.1179. Found: 406.1192.

### 1-((1′,4′-Dideoxy-1′,4′-imino-L-arabinitol)-4′-N-yl)-2,4-O-benzylidene-1-deoxy-D-erythritol-3-sulfate (31)

A mixture of 1,4-Dideoxy-1,4-imino-L-arabinitol (16) (80 mg, 0.6 mmol) and 2,4-O-benzylidene-D-erythritol-1,3-cyclic sulfate (7) (190 mg, 1.2equiv) were dissolved in dry MeOH (0.5 mL) and anhydrous K₂CO₃ (10 mg) was added. The mixture was stirred in a Caries tube in an oil-bath (75° C.) overnight. The solvent was removed under reduced pressure and column chromatography [CH₂Cl₂:MeOH, 5:1] of the crude product gave an amorphous solid (175 mg, 72%). ¹H NMR (CD₃OD): δ 7.52-7.31 (5H, m, Ar), 5.62 (1H, s, H-5), 4.53 (1H, dd, J=10.9, 5.2 Hz, H-4a), 4.28 (1H, m, H-2), 4.20 (1H, ddd, J=9.7, 5.1, 4.6 Hz, H-3), 4.14 (1H, br-s, H-2′), 4.03 (1H, m, H-1a), 3.98-3.84 (3H, m, H-3′, H-5′a, H-5′b), 3.81 (1H, dd, J=10.9, 10 Hz H-4b) 3.63 (1H, m, H-1′a), 3.55-3.42 (2H, m, H-1′b, H-4′), 3.38 (1H, m, H-1b); ¹³C NMR (100.6 MHz, CD₃OD): δ 138.66 (Cᵢₚₛₒ), 130.15 (Cₚₐᵣₐ), 129.23, 127.40 (4Cₒᵣₜₕₒ₊ₘₑₜₐ), 102.34 (C-5), 77.81 (C-4′), 77.52 (C-3′, C-2), 76.19 (C-2′), 70.27 (C-4), 68.92 (C-3), 62.68 (C-1′), 60.41 (C-5′), 58.61 (C-1); MALDI-TOF MS: m/e 428 (M⁺+Na), 406 (M⁺+H).

### 5.2.4.1 Example 4.1

### General Procedure for the Alternative Synthesis of Salacinol (1) (Schemes 10(a) to 10(c))

### 2,3,5-Tri-O-benzyl-1,4-dideoxy-1,4-[[(2S,3S)-2,4-di-(benzyloxy)-3-sulfoxy)butyl]-episulfoniumylidene]-D-arabinitol Inner Salt (42)

A mixture of the thioether 33²⁵ (270 mg, 0.64 mmol) and 2,4-Di-O-benzyl-1,3-cyclic sulfate (41)^{15,26} (280 mg, 0.77 mmol) in either acetone or HFIP (0.5 mL), containing anhydrous K₂CO₃ (16 mg, 0.10 mmol) was stirred in a sealed tube in an oil-bath (75-80° C.) for 14 h. The solvent was removed under reduced pressure and the residue was purified by column chromatography using (CH₂Cl₂:MeOH, 10:1) as eluant to give the title compound 42, as an amorphous solid (29 mg, 5%) in acetone and (229 mg, 45%) in HFIP. R_{f} 0.40 (CH₂Cl₂:MeOH, 10:1); [α]_{D}−26° (c 1.3, CHCl₃); ¹H NMR (CDCl₃): δ 7.38-7.05 (25H, m, Ar), 4.67 and 4.45 (2H, 2d, J_{A,B}=11.8 Hz, CH₂Ph), 4.60 and 4.45 (2H, 2d, J_{A,B}=9.5 Hz, CH₂Ph), 4.59 and 4.44 (2H, 2d, J_{A,B}=11.2 Hz, CH₂Ph), 4.58 (1H, dt, J_{2′,3′}=5.0 Hz, H-3′), 4.42 and 4.28 (2H, 2d, J_{A,B}=11.0 Hz, CH₂Ph), 4.36 (1H, m, H-2), 4.32 (1H, ddd, J=1.7, 4.1, 6.3 Hz, H-2′), 4.30 and 4.20 (2H, 2d, J_{A,B}=11.7 Hz, CH₂Ph), 4.23 (1H, m, H-3), 4.13 (1H, dd, J_{1′a,1′b}=13.4, J_{1′a,2′}=2.0 Hz, H-1′a), 4.05 (1H, d, J_{2,3}=13.3 Hz, H-1a), 4.00 (1H, dd, J_{4′a,4′b}=11.1, J_{3′,4′a}=2.7 Hz, H-4′a), 3.86 (1H, dd, J_{3′,4′b}=2.4, J_{4′a,4′b}=11.3 Hz, H-4′b), 3.71 (1H, brt, J=9.2 Hz, H-4), 3.69 (1H, dd, J_{1′b,2′}=3.8, J_{1′b,1′a}9.2 Hz, H-1′b), 3.60 (1H, dd, J_{1a,1b}=13.5, J_{1b,2}=3.8 Hz, H-1b), 3.51 (1H, dd, J_{5a,5b}=13.6, J_{4,5a}=9.7 Hz, H-5a), 3.49 (1H, dd, J_{4,5b}=9.7 Hz, H-5b); ¹³C NMR (CDCl₃): δ 137.97, 136.77, 136.71, 136.05 and 135.77 (5×Cᵢₚₛₒ Ph), 128.81-127.66 (25C, Ph), 83.14 (C-3), 81.65 (C-2), 74.59 (C-3′), 73.81, 73.53, 3.39, 72.12, 71.84 (5×CH₂Ph), 73.10 (C-2′), 68.79 (C-4′), 66.62 (C-5), 65.53 (C-4), 50.89 (C-1′), 48.07 (C-1). MALDI-TOF MS: m/e 785.41 (M⁺+H), 808.32 (M⁺+Na). Anal. Calcd for C₄₄H₄₈O₉S₂: C, 67.32; H, 6.16. Found: C, 67.36; H, 6.10.

### 2,3,5-Tri-O-benzyl-1,4-dideoxy-1,4-[[(2S,3S)-2,4-O-benzylidene-3 (sulfooxy)butyl]-episulfoniumylidene]-D-arabinitol (35)

A mixture of the thioether 33²⁵ (260 mg, 0.62 mmol) and 2,4-Di-O-benzylidene-1,3-cyclic sulfate (34)²⁵ (200 mg, 0.74 mmol) in either acetone or HFIP (0.5 ml) containing K₂CO₃ (13 mg, 0.09 mmol) was treated as described above to yield the title compound 35²⁵ as an amorphous solid (252 mg, 59% in acetone) and (406 mg, 94% in HFIP).

### 1,4-Anhydro-2,3,5-tri-O-(p-methoxybenzyl)-4-thio-D-arabinitol (43)

To an ice cold mixture of 1,4-anhydro-4-thio-D-arabinitol 38²⁵ (0.98 g, 6.52 mmol) and 60% NaH (1.56 g, 39.15 mmol, 6 equiv.) in THF (15 mL), a solution of p-methoxybenzyl chloride (4.59 g, 29.34 mmol, 4.5 equiv.) in THF (10 mL) was added over 30 min. The reaction mixture was allowed to attain room temperature and further stirred for 1 h before heating to 55° C. for 12 h. The reaction mixture was cooled and poured in to ice-water (150 mL) and extracted with Et₂O (150 mL). The organic solution was dried (Na₂SO₄) and concentrated. The product was purified by column chromatography [hexanes:EtOAc, 7:3] to give a colorless syrup (2.96 g, 87%). [α]_{D}+6° (c 1, CHCl₃); ¹H NMR (CDCl₃): δ 7.20-6.80 (12H, m, Ar), 4.55 (2H, s, CH₂Ph), 4.48 and 4.45 (2H, 2d, J_{A,B}=11.7 Hz, CH₂Ph), 4.42 and 4.39 (2H, 2d, J_{A,B}=12.0 Hz, CH₂Ph), 4.13 (1H, dd, J_{1,2}=4.6, J_{2,3}=9.1 Hz, H-2), 4.05 (1H, dd, J_{2,3}=J_{3,4}=3.7 Hz, H-3), 3.81 (3H, s, OCH₃), 3.79 (3H, s, OCH₃), 3.76 (3H, s, OCH₃), 3.64 (1H, dd, J_{5a,5b}=8.9, J_{4,5a}=7.5 Hz, H-5a), 3.50 (1H, ddd, J_{4,5b}=6.3 Hz, H-4), 3.45 (1H, dd, H-5b), 3.04 (1H, dd, J_{1a,1b}=11.4, J_{1a,2}=5.2 Hz, H-1a), 2.85 (1H, dd, H-1b). ¹³C NMR (CDCl₃): δ 159.24, 159.16 (3 Cₚₐᵣₐ), 130.31, 130.19, 130.01 (3Cᵢₚₛₒ), 129.48, 129.28, 129.22 (6 Cₒᵣₜₕₒ), 113.80, 113.74 (6 Cₘₑₜₐ), 84.77 (C-3), 84.70 (C-2), 72.66, 71.49, 71.20 (3×CH₂Ph), 72.15 (C-5), 55.24 (3×OCH₃), 48.96 (C-4), 33.07 (C-1). Anal. Calcd for C₂₉H₃₄O₆S: C, 68.21; H, 6.71. Found: C, 67.99; H, 6.69.

### 2,3,5-Tri-O-p-Methoxybenzyl-1,4-dideoxy-1,4-[[(2S,3S)-2,4-benzylidenedioxy-3-(sulfooxy)butyl]-episulfoniumylidene]-D-arabinitol Inner Salt (44)

A mixture of the thioether 43 (1.50 g, 2.94 mmol), and the cyclic sulfate 34 (0.96 g, 1.2 equiv) in HFIP (2.5 mL) containing anhydrous K₂CO₃ (30 mg) was stirred in a sealed tube in an oil-bath (55° C.) overnight. TLC analysis (CH₂Cl₂:MeOH, 10:1) showed that the thioether 43 was completely consumed. The solvent was removed under reduced pressure and the product was purified by column chromatography (gradient of CH₂Cl₂ to CH₂Cl₂:MeOH, 10:1) to give compound 13 (2.3 g, 100%) as a colorless foam. [α]_{D}−10.5° (c 1.1, CH₂Cl₂); ¹H NMR (CD₂Cl₂): δ 7.51-6.81 (17H, m, Ph), 5.53 (1H, s, C₆H₅CH), 4.57 (1H, ddd, J_{2′,3′}=J_{3′,4′ax}=10.0, J_{3′,4′eq}=5.5 Hz, H-3′), 4.49 (1H, dd, J_{4′ax,4′eq}=10.8 Hz, H-4′eq), 4.44 (2H, s, CH₂Ph), 4.42-4.39 (1H, m, H-2), 4.39 and 4.29 (2H, 2d, J_{A,B}=11.4 Hz, CH₂Ph), 4.33 (1H, dd, J_{1′a,1′b}=13.4, J_{1′a,2′}=2.6 Hz, H-1′a), 4.29-4.26 (1H, m, H-3), 4.26 (1H, ddd, H-2′), 4.19 and 4.09 (2H, 2d, J_{A,B}=11.5 Hz, CH₂Ph), 4.03 (1H, br d, J_{1a,2}<1 Hz, H-1a), 3.96-3.89 (2H, m, H-4, H-1′b), 3.80 (3H, s, OCH₃), 3.79 (3H, s, OCH₃), 3.78 (3H, s, OCH₃), 3.77 (1H, dd, H-4′ax), 3.63 (1H, dd, J_{1a,1b}=13.3, J_{1b,2}=3.8 Hz, H-1b), 3.58 (1H, dd, J_{5a,5b}=9.9, J_{4,5a}=8.5 Hz, H-5a), 3.49 (1H, dd, J_{4,5b}=7.3 Hz, H-5b); ¹³C NMR (CD₂Cl₂): δ 160.30, 160.23, 159.97, 137.20 and 130.27-126.61 (21×C, Ph), 114.45, 114.36 and 114.18 (3×Cᵢₚₛₒ, OMBn), 101.96 (PHCH), 83.29 (C-3), 82.37 (C-2), 76.76 (C-2′), 73.36, 72.43, and 72.14 (3×CH₂Ph), 69.50 (C-4′), 66.71 (C-5), 66.55 (C-4), 66.45 (C-3′), 55.61 (3C, 3×OCH₃), 49.55 (C-1′), 48.48 (C-1). Anal. Calcd for C₄₀H₄₆O₁₂S₂: C, 61.36; H, 5.92. Found: C, 61.13; H, 6.00.

1,4-Dideoxy-1,4-[[(2S,3S)-2,4-dihydroxy-3-(sulfooxy)butyl]-episulfoniumylidene]-D-arabinitol Inner Salt (1). Compound 13 (2.30 g, 2.94 mmol) was dissolved in trifluoroacetic acid (24 mL) and while stirring, water (2.4 mL) was added. The mixture was stirred at room temperature for 0.5 h. The solvent was removed under reduced pressure and the gummy residue was washed with CH₂Cl₂ (3×20 mL). Water (15 mL) was added to dissolve the crude product, and then evaporated under reduced pressure to remove the traces of acid left. Salacinol 1 (0.67 g, 68%) was crystallized from MeOH. The mother liquor was concentrated and purified by column chromatography (EtOAc:MeOH:H₂O, 7:3:1) to give more Salacinol 1 as a white solid (0.18 g, 18%).

### 5.2.5 Example 5

### General Procedure for the Deprotection of the Protected Sulfonium Sulfates (Schemes 7-10) and Ammonium Sulfates (Schemes 13-14)

The protected compound was dissolved in AcOH:H₂O, 4:1 (3 mL) and stirred with Pd—C (80 mg) under H₂ (52 psi). After 60 h the reaction mixture was filtered through a pad of Celite, which was consequently washed with MeOH. The combined filtrates were concentrated and the residue was purified by column chromatography.

### 1-((1′,4′-Anhydro-4′-thio-D-arabinitol)-4′-S-yl)-1-deoxy-L-erythritol-3-sulfate (1)

Column chromatography [CHCl₃:MeOH:H₂O, 7:3:1] of the crude product gave an amorphous solid (67%). [α]_{D}+2.1° (c 0.48, MeOH); ¹H NMR (pyridine-d5): δ 5.25 (1H, ddd, J=7.4, 3.8, 3.6 Hz, H-3), 5.14-5.09 (2H, m, H-3′, H-2′), 5.00 (1H, m, H-2), 4.78 (1H, dd, J=13.0, 4.9 Hz H-1a), 4.70 (1H, m, H-4′), 4.63 (1H, dd, J=13.0, 4.0 Hz H-1b), 4.61 (1H, dd, J=11.8, 3.7 Hz H-4a)4.53 (2H, m, H-5′a, H-5′b), 4.38 (1H, dd, J=11.8, 3.8 Hz H-4b), 4.32 (2H, br-s, H-1′a, H-1′b); ¹³C NMR (100.6 MHz, pyridine-d5): δ 79.14 (C-3), 79.06 (C-3′), 78.18 (C-2′), 72.30 (C-4′), 67.44 (C-2), 62.05 (C-4), 59.98 (C—5′), 52.46 (C-1), 50.35 (C-1′). HRMS. Calcd for C₉H₁₈O₉S₂ (M+H): 335.0471. Found: 335.0481.

### 1-((1′,4′-Anhydro-4′-thio-D-arabinitol)-4′-S-yl)-1-deoxy-D-crythritol-3-sulfate (23)

Column chromatography [CHCl₃:MeOH:H₂O, 7:3:1] of the crude product gave an amorphous solid (59%). [α]_{D}−35.6° (c 0.86, MeOH); ¹H NMR (pyridine-d5): δ 5.19 (1H, ddd, J=8.0, 4.1, 3.6 Hz, H-3), 5.17-5.12 (2H, m, H-2′, H-3′), 5.00 (1H, ddd, J=8.0, 5.3, 4.1 Hz, H-2), 4.83 (1H, dd, J=13.0, 5.1 Hz H-1a), 4.78 (1H, m, H-4′), 4.65 (1H, dd, J=11.9, 3.8 Hz H-4a), 4.64-4.57 (2H, m, H-5′a, H-5′b), 4.53 (1H, dd, J=13.0, 4.1 Hz H-1b), 4.40 (1H, dd, J=11.9, 3.8 Hz H-4b), 4.29 (1H, dd, J=12.7, 3.9 Hz H-1′a), 4.17 (1H, dd, J=12.7, 2.6 Hz H-1′b); ¹³C NMR (100.6 MHz, pyridine-d5): δ 79.46 (C-3), 79.38 (C-3′), 78.94 (C-2′), 71.94 (C-4′), 67.52 (C-2), 62.02 (C-4), 60.26 (C-5′), 52.64 (C-1), 51.01 (C-1′). HRMS. Calcd for C₉H₁₈O₉S₂ (M+H): 335.0471. Found: 335.0486.

### 1-((1′,4′-Anhydro-4′-thio-L-arabinitol)-4′-S-yl)-1-deoxy-D-erythritol-3-sulfate 25)

Column chromatography [CHCl₃:MeOH:H₂O, 7:3:1] of the crude product gave an amorphous solid (80%). [α]_{D}+1.1° (c 1.5, MeOH); ¹H NMR (pyridine-d5): δ 5.23 (1H, ddd, J=7.4, 3.8, 3.7 Hz, H-3), 5.11(1H, m, H-3′), 5.10 (1H, m, H-2′), 4.98 (1H,m, H-2), 4.76 (1H, dd, J=11.7, 3.7 Hz H-1a), 4.70 (1H, m, H-4′), 4.63 (1H, dd, J=11.7, 3.8 Hz H-1b), 4.60 (1H, dd, J=11.8, 3.7 Hz H-4a)4.51 (2H, m, H-5′a, H-5′b), 4.35 (1H, dd, J=11.8, 4.0 Hz H-4b), 4.31 (2H, m, H-1′a, H-1′b); ¹³C NMR (100.6 MHz, pyridine-d5): δ 79.38 (C-3, C-2′), 78.41 (C-3′), 72.51 (C-4′), 67.63 (C-2), 62.23 (C-4), 60.21 (C-5′), 52.60 (C-1), 50.57 (C-1′). HRMS. Calcd for C₉H₁₈O₉S₂ (M+H): 335.0471. Found: 335.0466.

### 1-((1′,4′-Dideoxy-1′,4′-imino-D-arabinitol)-4′-N-yl)-1-deoxy-L-erythritol-3-sulfate (2)

Column chromatography [CHCl₃:MeOH:H₂O, 7:3:1] of the crude product gave an amorphous solid (64%). ¹H NMR (CD₃OD): δ 4.26-4.20 (2H, m H-2, H-3), 4.15 (1H, m, H-2′), 3.98 (1H,br-s, H-3′), 3.94-3.87 (3H,m, H-5′a, H-5b′, H-4a), 3.81 (1H, dd, J=12.0, 3.5 Hz H-4b), 3.74-3.62 (2H, m, H-1a, H-1′a), 3.49-3.42 (1H, m, H-1′b), 3.40-3.35 (1H, m, H-4′), 3.15 (1H, m, H-1b); ¹³C NMR (100.6 MHz, CD₃OD): δ 81.17 (C-3), 78.27 (C-3′), 77.86 (C-4′), 76.19 (C-2′), 68.07 (C-2), 62.57 (C-1′), 61.67(C-4), 60.72 (C-1, C-5′). HRMS. Calcd for C₉H₁₈O₉SN (M+H): 318.0859. Found: 318.0863.

### 1-((1′,4′-Dideoxy-1′,4′-imino-L-arabinitol)-4′-N-yl)-1-deoxy-D-erythritol-3-sulfate (32)

Column chromatography [CHCl₃:MeOH:H₂O, 7:3:1] of the crude product gave an amorphous solid (77%). ¹H NMR (CD₃OD): δ 4.25 (1H, m H-2), 4.23(1H, m, H-3), 4.16 (1H, br-s, H-2′), 3.99 (1H,br-s, H-3′), 3.94-3.87 (3H,m, H-5′a, H-5b′, H-4a), 3.81 (1H, dd, J=12.1, 3.6 Hz H-4b), 3.77-3.64 (2H, m, H-1a, H-1′a), 3.55-3.39 (2H, m, H-1′b, H-4′), 3.22 (1H, m, H-1b); ¹³C NMR (100.6 MHz, CD₃OD): δ 81.18 (C-3), 78.23 (C-3′, C-4′), 76.10 (C-2′), 68.05 (C-2), 62.66 (C-1′), 61.88(C-4), 60.49 (C-1, C-5′). HRMS. Calcd for C₉H₁₈O₉SN (M+H): 318.0859. Found: 318.0856.

### 5.2.6 Example 6

### General Procedure for the Alternative Synthesis of Blintol (3) (Schemes 12a-12f)

### 1,2,3,5-Tetra-O-acetyl-L-xylofuranose (49)

L-Xylose (5.00 g, 33.3 mmol), boric acid (4.50 g, 73.2 mmol), and glacial acetic acid (100 mL) were added into a 250 mL round bottom flask. The mixture was stirred at 80° C. until L-xylose and boric acid were dissolved in acetic acid. Acetic anhydride (50 mL) was added and the reaction mixture was stirred at 75° C. for 4 h. Analysis by TLC (EtOAc: MeOH: H₂O, 10:3:1) showed that the L-xylose had been completely consumed. MeOH was then added to the reaction mixture, and the reaction mixture was concentrated to give a dark, orange-brown syrup. To this syrup, acetic anhydride (50 mL) and pyridine (50 mL) were added and the reaction mixture was stirred at room temperature for 4 h. The orange-brown mixture was pour into crushed ice and was extracted with Et₂O (100 mL). The organic layer was washed with saturated aqueous NaHCO₃ (50 mL), aqueous HCl, water, and saturated NaCl, dried over MgSO₄ and concentrated to a yellow syrup. Purification by column chromatography on silica gel (Hexane:EtOAc, 2:1) yielded the tetra-O-acetylxylofuranose 49 (9.01 g, 85%) as a colorless syrup (α:β ratio 1:23). Data for the β (major) isomer.

¹H NMR (CDCl₃): δ 6.08 (1H, s, H-1), 5.35 (1H, dd, J_{2,3}=1.7, J_{3,4}=5.6 Hz, H-3), 5.18 (1H, d, J_{1,2}<1 Hz, H-2), 4.62 (1H, dd, J_{4,5a}<1, J_{4,5b}=12.1 Hz, H-4), 4.22 (2H, m, H-5a, H-5b), 2.10, 2.09, 2.08, and 2.04 (12H, 4 s, COCH₃). ¹³C NMR (CDCl₃): d 170.71, 169.69, 169.52, 169.43 (4×C═O, OAc), 99.01 (C-1), 80.03 (C-2), 79.58 (C-3), 74.43 (C-4), 62.54 (C-5), 21,33, 20.97, 20.82, 20.68 (4×CH₃, OAc). Anal. Calcd for C₁₃H₁₈O₉: C, 49.06; H, 5.70. Found: C, 48.93; H, 5.84.

### 4-Pentenyl-2,3,5-tri-O-acetyl-L-xylofuranoside (50)

Tetra-O-acetylxylofuranose 49 (5.00 g, 17.7 mmol), CH₂Cl₂ (100 mL), 4-penten-1-ol (9.1 mL, 88 mmol), and crushed molecular sieves (4 Å, 2 g) were added to a 250 mL round bottom flask and cooled to 0° C. Boron trifluoride (11 mL, 88 mmol) was added to the reaction mixture and the mixture was stirred at 0° C. for 2 h. The temperature was raised to room temperature and the mixture was stirred for 1 h. Analysis by TLC (Hexane: EtOAc, 2:1) showed that the majority of the starting material had been consumed. The reaction mixture was poured into ice/NaHCO₃ mixture, extracted with Et₂O (100 mL), and dried over MgSO₄. The reaction mixture was concentrated to a dark, orange-brown syrup. Purification by column chromatography on silica gel (Hexane:EtOAc, 2:1) yielded the pentenyl glycosides 50 (3.28 g, 60%) as a colorless syrup (α:β ratio 1:23).

Data for the β (major) isomer. ¹H NMR (CDCl₃): δ 5.78 (1H, dddd, J_{4′,5b′}=23.6, J_{4′,5a′}=17.1, J_{3a′,4′}=3.6, J_{3b′,4′}=13.3 Hz, H-4′), 5.30 (1H, dd, J_{2,3}=1.5, J_{3,4}=6.0 Hz, H-3), 5.07 (1H, s, J_{1,2}<1 Hz, H-2), 4.99 (1H, 2 ddd, J_{3′a,5′a}=1.7, J_{3′b,5′a}=1.7, J_{5′b,5′a}=3.5 Hz, H-5a′), 4.94 (1H, s, H-1), 4.93 (1H, m, H-5′b), 4.55 (1H, ddd, J_{4,5a}=5.3, J_{4,5b}=7.3 Hz, H-4), 4.24 (1H, dd, J_{5a,5b}=11.5, H-5a), 4.18 (1H, dd, H-5b), 3.69 (1H, ddd, J_{1′a,2′a}=6.7, J_{1′a,2′b}=6.7, J_{1′a,1′b}=13.3 Hz, H-1′a), 3.40 (1H, ddd, J_{1′b,2′a}=6.4, J_{1′b,2′b}=6.4 Hz, H-1′b), 2.07 (6H, s, 2×COCH₃), 2.04 (3H, s, COCH₃), 2.04 (2H, m, H-3′a, H-3′b), 1.65 (2H, m, H-2′a, H-2′b). ¹³C NMR (CDCl₃): δ 170.72, 170.11, and 169.74 (3×C═O, OAc), 138.22 (C-4′), 115.13 (C-5′), 106.08 (C-1), 80.92 (C-2), 78.17 (C-4), 75.11 (C-3), 67.77 (C-1′), 63.42 (C-5), 30.34 (C-3′), 28.78 (C-2′), 20.99, 20.93, and 20.81(3×CH₃, OAc). Anal. Calcd for C₁₆H₂₄O₈: C, 55.81; H, 7.02. Found: C, 55.99; H, 7.19.

### 4-Pentenyl-L-xylofuranoside (51)

The pentenyl glycoside 50 (3.28 g, 9.52 mmol) was dissolved into MeOH (50 mL) in a 250 mL round bottom flask. NaOMe in MeOH (0.02 M) was added to the reaction mixture and the mixture was stirred at room temperature for 1 h. Analysis by TLC (CH₂Cl₂: MeOH, 10:1) showed the starting material had been consumed. Rexyn® 101 (H) resin was added to the reaction mixture to adjust the PH to 7. The reaction mixture was then filtered and the filtrate was concentrated to give a light brown syrup. Purification by column chromatography on silica gel (CH₂Cl₂: MeOH, 10:1) yielded the pentenyl glycosides 51 (1.97 g, 95%) as a colorless syrup (α:β ratio 1:23).

Data for the β (major) isomer. ¹H NMR (CD₃OD): δ 5.75 (1H, m, H-4′), 5.03 (1H, m, H-5′a), 4.96 (1H, m, H-5′b), 4.86 (1H, s, J_{1,2}<1 Hz, H-1), 4.24 (1H, ddd, J_{4,5a}=5.0, J_{4,5b}=6.6, J_{3,4}=5.1 Hz, H-4), 4.08 (1H, dd, J_{2,3}=2.0, H-3), 4.03 (1H, br.s, H-2), 3.83 (1H, dd, J_{5a,5b}=11.6, H-5a), 3.79 (1H, m, H-1′a), 3.74 (1H, m, H-5b), 3.43 (1H, m, H-1′b), 2.17 (2H, m, H-3′a, H-3′b), 1.68 (2H, m, H-2′a, H-2′b). ¹³C NMR (CD₃OD): δ 138.23 (C-4′), 114.17 (C-5′), 109.62 (C-1), 82.71 (C-4), 81.01 (C-2), 76.31 (C-3), 67.42 (C-1′), 61.49 (C-5), 32.20 (C-3′), 28.78 (C-2′). Anal. Calcd for C₁₀H₁₈O₅: C, 55.03; H, 8.31. Found: C, 55.30; H, 8.44.

### 4-Pentenyl-2,3,5-tri-O-p-methoxybenzyl-L-xylofuranoside (52)

In a 250 mL flask NaH (4.38 g, 0.11 mol) and DMF (80 mL) were added and cooled to 0° C. The pentenyl glycoside 51 (3.00 g, 13.7 mmol) was dissolved in DMF (10 mL) and the solution was added dropwise to the NaH/DMF mixture. After the addition, the reaction mixture was stirred at 0° C. for 2 h. The temperature was then raised to room temperature and the mixture was stirred for 1 h. p-Methoxybenzyl chloride (15 mL, 0.11 mol) dissolved in DMF (10 mL) was then added dropwise to the reaction mixture. The mixture was stirred at room temperature for 2 h after the addition. The reaction mixture was quenched with ice water, extracted with Et₂O (100 mL), washed with H₂O (8×20 mL portions), and dried over MgSO₄. The mixture was concentrated to give a orange-brown syrup. Purification by column chromatography on silica gel (Hexane:EtOAc, 4:1) yielded the pentenyl glycosides 52 (7.30 g, 92%) as a colorless syrup (α:β ratio 1:23).

Data for the β (major) isomer. ¹H NMR (CDCl₃): δ 7.25-6.85 (12H, m, Ar), 5.83 (1H, dddd, J_{4′,5b′}=6.6, J_{4′,5a′}=16.9, J_{3′a,4′}=6.8, J_{3′b,4′}=10.4 Hz, H-4′), 5.03 (1H, dddd, J_{3′a,5′a}=1.7, J_{3′b,5′a}=5.5, J_{5′b,5′a}=3.5 Hz, H-5a′), 4.98 (1H, br.s, J_{1,2}=1.8 Hz, H-1), 4.97 (1H, m, H-5′b), 4.49 (6H, m, 3×CH₂Ph), 4.41 (1H, m, H-4), 4.02 (1H, dd, J_{2,3}=2.3, J_{3,4}=5.8 Hz, H-3), 3.97 (1H, br.t, H-2), 3.81 (6H, s, 2×OCH₃), 3.80 (3H, s, OCH₃), 3.76 (1H, m, H-1′a), 3.72 (1H, dd, J_{4,5a}=4.7, J_{5a,5b}=10.3 Hz, H-5a), 3.67 (1H, dd, J_{4,5b}=7.3 Hz, H-5b), 3.42 (1H, m, H-1′b), 2.12 (2H, m, H-3′a, H-3′b), 1.68 (2H, m, H-2′a, H-2′b). ¹³C NMR (CDCl₃): δ 159.60-113.91 (12 C_{Ar}), 138.51 (C-4′), 114.03 (C-5′), 107.38 (C-1), 87.02 (C-2), 81.83 (C-3), 80.04 (C-4), 73.32, 71.93, 71.81 (3×CH₂Ph), 69.78 (C-5), 67.94 (C-1′), 55.52 (OCH₃), 30.61 (C-3′), 28.98 (C-2′). Anal. Calcd for C₃₄H₄₂O₈: C, 70.57; H, 7.32. Found: C, 70.44; H, 7.48.

### 2,3,5-Tri-O-p-methoxybenzyl-L-xylofuranose (53)

In a 500 mL round bottom flask pentenyl glycosides 52 (7.00 g, 12.1 mmol) were dissolved in CH₃CN (180 mL). H₂O (20 mL) was added and the mixture was cooled to 0° C. N-Bromosuccinimide (5.38 g, 30.2 mmol) was added to the reaction mixture and the reaction mixture was stirred at 0° C. for 1 h. Analysis by TLC (Hexane: EtOAc, 2:1) showed that the starting material had been completely consumed. Na₂S₂O₃ 5H₂O (15 g, 60 mmol) dissolved in H₂O (60 mL) was then added and the mixture was stirred for 20 min. The mixture was then concentrated to give a dark orange syrup. The syrup was dissolved in EtOAc (150 mL), washed with H₂O, saturated NaCl, and dried over MgSO₄. The mixture was then concentrated to give a dark brown syrup. Purification by column chromatography on silica gel (Hexane:EtOAc, 1:1) yielded the p-methoxybenzyl xylofuranoses 53 (5.52 g, 90%) as a colorless syrup (α:β ratio 1:2).

Data for the β (major) isomer. ¹H NMR (CDCl₃): δ 7.25-6.80 (12H, m, Ar), 5.20 (1H, br.s, J_{1,2}=1.8 Hz, H-1), 4.55-4.40 (6H, m, 3×CH₂Ph), 4.34(1H, ddd, J_{4,5b}=5.0, J_{4,5a}=4.1, J_{3,4}=5.4 Hz, H-4), 4.05 (1H, dd, J_{2,3}=2.8 Hz, H-3), 3.95 (1H, br.d, J_{1,2}<1 Hz H-2), 3.82, 3.81, 3.80(9H, 3×s, 3×OCH₃), 3.68 (2H, m, H-5a, H-5b). ¹³C NMR (CDCl₃): δ 159.60-113.50 (12 C_{Ar}), 101.68 (C-1), 86.24 (C-2), 80.91 (C-3), 79.83 (C-4), 73.32, 72.33, 71.48 (3×CH₂Ph), 68.31 (C-5), 55.22 (OCH₃). Anal. Calcd for C₂₉H₃₄O₈: C, 68.22; H, 6.71. Found: C, 68.17; H, 6.65.

### 2,3,5-Tri-O-p-methoxybenzyl-L-xylitol (54)

The p-methoxybenzyl xylofuranoses 53 (5.50 g, 10.8 mmol) were dissolved in THF (10 mL) and MeOH (50 mL) was then added. NaBH₄ was added portionwise to the reaction mixture at room temperature until the TLC analysis (Hexane:EtOAc, 1:1) showed that the starting material had been consumed. The mixture was concentrated to give a light yellow solid. This solid was dissolved in EtOAc (150 mL), washed with water, saturated aqueous NaCl, dried over MgSO₄, and concentrated to give a light yellow syrup. Purification by column chromatography on silica gel (Hexane:EtOAc, 1:1) yielded the p-methoxybenzyl xylitol 54 as a colorless syrup (4.62 g, 84%).

[α]_{D}+7.25 (c 2.8, CHCl₃). ¹H NMR (CDCl₃): δ 7.20-6.80 (12H, m, Ar), 4.58, 4.43 (2H, 2d, J_{A,B}11.2 Hz, CH₂Ph), 4.54 (2H, 2d, J_{A,B}11.2 Hz, CH₂Ph), 4.44, 4.39 (2H, 2d, J_{A,B}=11.7 Hz, CH₂Ph), 4.02 (1H, ddd, J_{2,3} 1.9, J_{1a,2}=6.4, J_{1b,2}=6.2 Hz, H-2), 3.80 (9H, s, 3×OCH₃), 3.75 (2H, m, H-4, H-5a), 3.66 (1H, dd, J_{3,4}=6.4 Hz, H-3), 3.63 (1H, m, H-5b), 3.46 (1H, dd, J_{1a,1b}=9.4 Hz, H-1a), 3.37 (1H, dd, H-1b). ¹³C NMR (CDCl₃): δ 159.60-113.50 (12 C_{Ar}), 78.32 (C-3), 77.01 (C—5), 73.88, 73.08, 72.10 (3×CH₂Ph), 71.23 (C-1), 68.79 (C-2), 60.81 (C-4), 55.53 (OCH₃). Anal. Calcd for C₂₉H₃₆O₈: C, 67.95; H, 7.08. Found: C, 67.85; H, 7.12.

### 2,3,5-Tri-O-p-methoxybenzyl-1,4-di-O-methanesulfonyl-L-xylitol (55)

In a 250 mL round bottom flask, methanesulfonyl chloride (5.3 mL, 68 mmol), pyridine (6 mL, 68 mmol), and CH₂Cl₂ (50 mL) were cooled to 0° C. The p-methoxybenzyl xylitol (54, 3.50 g, 6.84 mmol) in CH₂Cl₂ (50 mL) was then added dropwise to the methanesulfonyl chloride/pyridine mixture. After the addition was completed, the temperature was raised to room temperature and the mixture was stirred for 3 h. The reaction mixture was then poured onto crushed ice, extracted with EtOAc (150 mL), washed with water, saturated aqueous NaCl, dried over MgSO₄, and was concentrated to give a light yellow syrup. Purification by column chromatography on silica gel (Hexane:EtOAc, 1:1) yielded the methanesulfonyl xylitol 55 as a colorless syrup (3.28 g, 72%).

[α]_{D}−16.2 (c 5.6, CHCl₃). ¹H NMR (CDCl₃): δ 7.20-6.80 (12H, m, Ar), 4.92 (1H, ddd, J_{2,3}=9.2, J_{1a,2}=3.6, J_{1b,2}=6.1 Hz, H-2), 4.60, 4.43 (2H, 2d, J_{A,B}=11.3 Hz, CH₂Ph), 4.57 (2H, 2d, J_{A,B}=11.3 Hz, CH₂Ph), 4.41, 4.33 (2H, 2d, J_{A,B}=11.1 Hz, CH₂Ph), 4.36 (1H, dd, J_{4,5a}=5.6, J_{5a,5b}=11.0 Hz, H-5a), 4.31 (1H, dd, J_{4,5b}=4.2 Hz, H-5b), 3.83 (1H, m, H-4), 3.80, 3.79, 3.78 (9H, 3s, 3×OCH₃), 3.78 (1H, m, H-3), 3.56 (1H, dd, J_{1a,1b}=11.2 Hz, H-1a), 3.54 (1H, dd, H-1b), 2.99, 2.92 (6H, 2 s, 2×OSO₂CH₃). ¹³C NMR (CDCl₃): δ 159.60-113.50 (12 C_{Ar}), 80.32 (C-2), 75.63 (C-3), 75.24 (C-4), 74.11, 72.83, 72.69 (3×CH₂Ph), 68.43 (C-5), 68.41 (C-1), 55.12 (OCH₃), 38.5, 37.1 (2×OSO₂CH₃). Anal. Calcd for C₃₁H₄₀O₁₂S₂: C, 55.67; H, 6.03. Found: C, 55.45; H, 6.13.

### 1,4-Anhydro-2,3,5-tri-O-p-methoxybenzyl-4-seleno-D-arabinitol (56)

In a 250 mL round bottom flask, selenium metal (0.61 g, 7.7 mmol) and 95% EtOH (50 mL) were added. NaBH₄ was then added portionwise at room temperature until the color of the reaction mixture changed from black to white. The dimesylate 55 (3.28 g, 4.91 mmol) dissolved into THF (10 mL) was then added to the reaction mixture and the mixture was heated and stirred at 60° C. for 12 h. The mixture was then concentrated to give a dark orange-red syrup. This solid was dissolved into Et₂O (100 mL), washed with water, saturated aqueous NaCl, dried over MgSO₄, and was concentrated to give a light yellow syrup. Purification by column chromatography on silica gel (Hexane:EtOAc, 4:1) yielded the selenoarabinitol 56 as a colorless syrup (2.27 g, 83%).

[α]_{D}+17.83 (c 1.5, CHCl₃). ¹H NMR (CDCl₃): δ 7.20-6.80 (12H, m, Ar), 4.58, 4.52 (2H, 2d, J_{A,B}=11.4 Hz, CH₂Ph), 4.48, 4.44 (2H, 2d, J_{A,B}=11.6 Hz, CH₂Ph), 4.45, 4.42 (2H, 2d, J_{A,B}=11.7 Hz, CH₂Ph), 4.16 (1H, ddd, J_{2,3}=5.2, J_{1a,2}=5.1, J_{1b,2}=5.4 Hz, H-2), 4.00 (1H, dd, J_{3,4}=4.8 Hz, H-3), 3.81 (1H, m, H-5a), 3.81 (6H, s, 2×OCH₃), 3.80 (3H, s, OCH₃), 3.72 (1H, m, H-4), 3.48 (1H, dd, J_{4,5b}=7.2, J_{5a,5b}=9.3 Hz, H-5b), 3.06 (1H, dd, H-1a), 2.92 (1H, dd, H-1b). ¹³C NMR (CDCl₃): δ 159.20-113.50 (12 C_{Ar}), 85.73 (C-2), 85.33 (C-3), 72.89 (C-5), 72.83, 72.01, 71.42 (3×CH₂Ph), 55.22 (OCH₃), 42.38 (C-4), 23.91 (C-1). Anal. Calcd for C₂₉H₃₄O₆SC: C, 62.47; H, 6.15. Found: C, 62.39; H, 6.25.

### 2,4-O-Benzylidene-L-erythritol-1,3-cyclic Sulfate (57)

The cyclic sulfate 62, prepared according to literature procedures,²⁵ (13.5 g, 37.0 mmol) was dissolved in EtOAc (120 mL) in a 500 mL round bottom flask. Pd on activated carbon (200 mg, 10% palladium) was added to the solution and H₂ was bubbled through the solution with stirring at room temperature for 48 h. Periodic analysis by TLC (Hexane: EtOAc, 1:1) showed that the reaction proceeded smoothly until the cyclic sulfate 62 had been consumed. The Pd was removed by filtration and the solvent was evaporated to yield the deprotected cyclic sulfate 63 as a white solid (6.82 g, quantitative yield). The cyclic sulfate 63 was used directly without further purification. The cyclic sulfate 63 and pyridinium p-toluenesulfonate (500 mg) were dissolved in CH₂Cl₂ (20 mL) in a 250 mL round bottom flask and PhCH(OMe)₂ (37 mL, 0.26 mol) was added. The solution was heated to 60° C. on a rotary evaporator under vacuum for 1 h. Analysis by TLC (Hexane: EtOAc, 1:1) showed that the cyclic sulfate 57 had been consumed. The mixture was dissolved in EtOAc (100 mL), washed with saturated aqueous NaCl (20 mL), dried over MgSO₄, and was concentrated to give a colorless syrup. Purification by column chromatography on silica gel (Hexane:EtOAc, 1:1) yielded the cyclic sulfate 57 as a white solid (7.14 g, 71%). This material was identical in all respects to that obtained previously²⁵ using L-glucose.

### 1,3-Di-O-benzyl-D-erythritol (60)—Alternative Procedure

In a 250 mL flask, 2,4-O-Benzylidene-1,3-di-O-benzyl-D-erythritol (59, 36.6 g, 93.7 mmol) and 50% aqueous TFA solution (100 mL) were added. The reaction mixture was stirred at room temperature for 0.5 h. Analysis by TLC (Hexane: EtOAc, 2:1) showed the starting material had been consumed. The reaction mixture was cooled to 0° C. and 50% aqueous KOH solution (50 mL) was added. The reaction mixture was stirred at 0° C. for additional 0.5 h, extracted with EtOAc (200 mL), and dried over Na₂SO₄. The mixture was concentrated to give a brown syrup. Purification by column chromatography on silica gel (Hexane: EtOAc, 1:1) yielded the erythritol 60 (17.6 g, 60%) as a colorless syrup. This material was identical in all respects to that obtained previously²⁶ using aqueous acetic acid.

### 2,3,5-Tri-O-p-Methoxybenzyl-1,4-dideoxy-1,4-[[(2S,3S)-2,4-benzylidenedioxy-3-(sulfooxy)butyl]-episelenoniumylidene]-D-arabinitol Inner Salt (64)

The seleno-D-arabinitol 56 (3.11 g, 5.59 mmol), the cyclic sulfate 57 (1.33 g, 4.88 mmol) and K₂CO₃ (160 mg, 1.16 mmol) were added to 1,1,1,3,3,3-hexafluoro-2-propanol (8.0 mL) and the mixture was stirred in a sealed tube with heating at 60-65° C. for 7 h. Periodic analysis by TLC (EtOAc: MeOH, 10:1) showed that the reaction proceeded smoothly until the selenoether had been consumed leaving some cyclic sulfate unreacted. The mixture was cooled and filtered through Celite with the aid of CH₂Cl₂. The solvents were removed and the residue was purified by column chromatography (gradient of EtOAc to EtOAc: MeOH, 10:1). The selenonium salt 64 (3.85 g, 95% based on selenoether 14) was obtained as a colorless foam. Analysis of the ¹H and ¹³C NMR spectra indicated that compound 64 was produced as a 7:1 mixture of isomers at the stereogenic selenium center. The major isomer was assigned to be the isomer with a trans relationship between C-5 and C-1′ by analogy to the results obtained previously for the corresponding benzyl-protected selenonium salt. For trans 64: ¹H NMR (600 MHz, CD₂Cl₂) δ 7.45-6.80 (17H, m, Ar), 5.58 (1H, s, C₆H₅CH), 4.51 (1H, dd, J_{2′,3′}=J_{3′,4′ax}=9.7, J_{3′,4′eq}=5.3 Hz, H-3′), 4.48 (1H, br s, H-2), 4.46 (1H, dd, J_{4′ax,4′eq}=10.5 Hz, H-4′eq), 4.41, 4.33 (2H, 2d, J_{A,B}=11.1 Hz, CH₂Ph), 4.57 (2H, 2d, J_{A,B}=11.3 Hz, CH₂Ph), 4.43 and 4.40 (2H, 2d, J_{A,B}=12.0 Hz, CH₂Ph), 4.39 and 4.26 (2H, 2d, J_{A,B}=11.4 Hz, CH₂Ph), 4.32 (1H, dd, J_{1′a,2′}=2.2 Hz, H-1′a), 4.27 (1H, br d, J_{2,3}=2.0 Hz, H-3), 4.25 and 4.19 (2H, 2d, J_{A,B}=10.8 Hz, CH₂Ph), 4.21 (1H, ddd, H-2′), 4.04 (1H, br d, J_{1,2}<1 Hz, H-1a), 4.03 (1H, br dd, J_{3,4}<1 Hz, H-4), 3.90 (1H, dd, J_{1′a,1′b}=12.2, J_{1′b,2′}=3.6 Hz, H-4), 3.78 (3H, s, OCH₃), 3.77 (1H, dd, H-4′ax), 3.77 (3H, s, OCH₃), 3.76 (3H, s, OCH₃), 3.55 (1H, dd, J_{1a,1b}=12.8, J_{1b,2}=2.9 Hz, H-1b), 3.54(1H, dd, J_{5a,5b}=9.7, J_{4,5a}=6.7 Hz, H-5a), 3.48 (1H, dd, J_{4,5b}=9.4 Hz, H-5b); ¹³C NMR (150 MHz, CDCl₂): δ

160.34, 160.09, 136.58 and 130.14-126.51 (21 C_{Ar}), 114.56, 114.47 and 114.70 (3×Cᵢₚₛₒ, OMBn), 102.17 (PHCH), 84.31 (C-3), 83.00 (C-2), 77.30 (C-2′), 73.37, 72.49, and 72.10 (3×CH₂Ph), 69.67 (C-4′), 67.75 (C-3′), 66.80 (2×C, C-4, C-5), 55.67 (3×C, 3×OCH₃), 48.73 (C-1′), 46.69 (C-1). Anal. Calcd for C₄₀H₄₆O₁₂SSe: C, 57.90; H, 5.59. Found: C, 57.87; H, 5.57.

### 1,4-Dideoxy-1,4-[[(2S,3S)-2,4-dihydroxy-3-(sulfooxy)butyl]episelenoniumylidene]-D-arabinitol Inner Salt (Blintol, 3)

The selenonium salts 64 (3.80 g, 4.58 mmol) were dissolved in cold trifluoroacetic acid (40 mL) to give a purple solution. Water (4.0 mL) was added and the reaction mixture was kept at room temperature for 0.5 h. The solvents were removed on a rotary evaporator and the residue was triturated with CH₂Cl₂ (4×50 mL), with each portion of solvent being decanted from the insoluble gummy product. The crude product was dissolved in water (50 mL) and filtered to remove a small amount of insoluble material. The aqueous filtrate was concentrated to a syrupy residue (1.84 g). Analysis by NMR spectroscopy indicated that the product was an isomeric mixture (7:1) of 3 with its stereoisomer at the selenium center. Recrystallization from MeOH gave pure 3 (1.09 g, 62%) in two crops. This material was identical in all respects to that obtained previously²⁶ using hydrogenolysis to remove the benzyl protecting groups. Purification of the mother liquor fractions by column chromatography (EtOAc: MeOH: H₂O, 6:3:1) gave a 3:2 mixture of 3 with its isomer (0.25 g, 14%) as a syrup.

### 5.2.7 Example 7

### Synthesis of Six-Membered Ring Analogues (Schemes 15 to 21)

General.

Optical rotations were measured at 23° C. Analytical thin-layer chromatography (TLC) was performed on aluminum plates precoated with Merck silica gel 60F-254 as the adsorbent. The developed plates were air-dried, exposed to UV light and/or sprayed with a solution containing 1% Ce(SO₄)₂ and 1.5% molybdic acid in 10% aq H₂SO₄ and heated. Compounds were purified by flash chromatography on Kieselgel 60 (230-400 mesh). Rexyn 101 was obtained from Fischer. ¹H and ¹³C NMR spectra were recorded on: Bruker AMX-400 NMR spectrometer at 400.13 MHz, Bruker AMX-600 NMR spectrometer at 600.13 MHz and Varian INOVA 500 NMR spectrometer at 499.97 MHz for ¹H. Chemical shifts are given in ppm downfield from TMS for those measured in CDCl₃, CD₃OD and CD₂Cl₂ and from 2,2-dimethyl-2-silapentane-5-sulfonate (DSS) for those spectra measured in D₂O. Chemical shifts and coupling constants were obtained from a first-order analysis of the spectra. Assignments were fully supported by two-dimensional ¹H,¹H(COSY), ¹H¹H (NOESY) and ¹H,¹³C (HMQC) experiments using standard Bruker or Varian pulse programs. Processing of the spectra was performed with standard UXNMR and WINNMR software (Bruker) or MestReC software (Varian).

The 1D-transient NOE experiments were performed by inverting the signal of interest with a 80 ms Gaussian selective pulse which was constructed from 1024 steps. Spectra were collected in difference mode by alternating the phase of the receiver gain during on- and off-resonance. The digitized signal was stored in a 32 K data set using a sweep width of 10 ppm, an acquisition time of 2.72 s, 128 scans, and 8 dummy scans. Processing of the spectra was accomplished by zero filling to 64 K followed by an exponential multiplication using a line width of 1 Hz. NOESY spectra were obtained with a mixing time of 500 or 800 ms.

MALDI mass spectra were obtained on a PerSeptive Biosystems, Voyager Del. time-of-flight spectrometer for samples dispersed in a 2,5-dihydroxybenzoic acid matrix. High resolution mass spectra were liquid secondary ion mass spectrometry (LSIMS), run on a Kratos Concept double focussing mass spectrometer at 10 000 RP, using a glycerin matrix or, in the case of compound 88a, with meta-NO₂-benzyl alcohol as the matrix. Solvents were distilled before use and were dried, as necessary. Solvents were evaporated under reduced pressure and below 50° C.

### 1,5-Anhydro-2,3,4-tri-O-benzyl-5-thioxylitol (74)

(a) Acetate Methanolysis: A mixture of 1,5-anhydro-2,3,4-tri-O-acetyl-5-thioxylitol 77 (0.125 g, 0.453 mmol) and 1M NaOMe in MeOH (0.6 mL, 0.6 mmol) in dry MeOH (10 mL) was stirred under N₂ overnight. The mixture was neutralized with excess Rexyn 101. The resin was removed by filtration and the organic phase was concentrated to give 1,5-anhydro-5-thioxylitol as a solid (59.6 mg, 88%). Mp 137-140° C.; ¹H NMR (D₂O): δ 3.65 (2H, m, J_{1eq,2}=J_{4,5eq}=4.5 Hz, J_{1ax,2}=J_{4,5ax}=10.9 Hz, H-2 and H-4), 3.15 (1H, t, J_{2,3}=J_{3,4}=9.1 Hz, H-3), 2.66 (2H, m, H-5eq and H-1 eq), 2.56 (2H, dd, J_{5ax,5eq}=J_{1ax,1eq}=13.6 Hz, H-5ax and H-1ax); ¹³C NMR (D₂O): δ 81.20 (C-3), 75.75 (2C, C-2 and C-4), 34.86 (2C, C-1 and C-5). Anal. Calcd for C₅H₁₀O₃S: C, 39.99; H, 6.71. Found: C, 39.68; H, 6.91.

(b) Benzylation: A mixture of 1,5-anhydro-5-thioxylitol (0.520 g, 3.47 mmol) and 60% NaH (0.744 g, 5 equiv) in DMF (50 mL) was stirred in an ice-bath for 1 h. A solution of BnBr (1.4 mL, 4 equiv) was added and the solution was stirred at RT overnight. The mixture was quenched with MeOH (8 mL), H₂O (100 mL) was added, and the solution was extracted with Et₂O (3×150 mL). The organic solution was dried over Na₂SO₄, concentrated, and the residue was purified by flash chromatography [hexanes:EtOAc, 20:1] to give 74 as a white solid (0.928 g, 64%). Mp 46-49° C.; ¹H NMR (CDCl₃): δ 7.36-7.24 (15H, m, Ar), 4.83 (2H, s, CH₂Ph), 4.69 (2H, d, J_{A,B}=11.4 Hz, CH₂Ph), 4.65 (2H, d, J_{A,B}=11.6 Hz, CH₂Ph), 3.63 (2H, m, J_{1eq,2}=J_{4,5eq}=4.2 Hz, J_{1ax,2}=J_{4,5ax}=11.0 Hz, H-4 and H-2), 3.31 (1H, t, J_{2,3}=J_{3,4}=8.9 Hz, H-3), 2.72 (2H, m, H-5eq and H-1 eq), 2.47 (2H, dd, J_{5ax,5eq}=J_{1ax,1eq}=13.4 Hz, H-5ax and H-1ax); ¹³C NMR (CDCl₃): δ 138.9, 138.37 (3Cᵢₚₛₒ), 128.42-127.51 (15C, Ar), 86.76 (C-3), 82.26 (2C, C-2 and C-4), 76.33 (CH₂Ph), 73.02 (2 CH₂Ph), 31.49 (2C, C-1 and C-5). Anal. Calcd for C₂₆H₂₈O₃S: C, 74.25; H, 6.71. Found: C, 74.16; H, 6.91.

### 1,5-Anhydro-2,3,4,6-tetra-O-benzyl-5-thio-D-glucitol (75)

(a) Acetate Methanolysis: To a solution of 1,5-anhydro-2,3,4,6-tetra-O-acetyl-5-thio-D-glucitol 78 (0.310 g, 0.89 mmol) in dry MeOH (20 mL) was added 1M NaOMe/MeOH (4 mL, 4 equiv), and the mixture was stirred under N₂ overnight. The mixture was neutralized with excess Rexyn 101 ion-exchange resin, the resin was removed by filtration, and the organic phase was concentrated. The residue was purified by flash chromatography [CHCl₃:MeOH, 5:2] to give 1,5-anhydro-5-thio-D-glucitol as a white solid (0.125 g, 78%). Mp 110-115° C.; [α]_{D}=+27.4 (c 1.2, MeOH); ¹H NMR (D₂O): δ 3.90 (1H, dd, J_{5,6a}=3.2 Hz, J_{6b,6a}=11.9 Hz, H-6a), 3.75 (1H, dd, J_{5,6b}=6.4 Hz, H-6b), 3.64 (1H, m, H-2), 3.48 (1H, dd, J_{4,5}=10.2 Hz, H-4), 3.19 (1H, t, J_{2,3}=J_{3,4}=9.1 Hz, H-3), 2.88 (1H, m, H-5), 2.71 (1H, dd, J_{1eq,2}=4.6 Hz, J_{1eq,1ax}=13.3 Hz, H-1 eq), 2.62 (1H, dd, J_{1ax,2}=11.0 Hz, H-1ax).

(b) Benzylation: To a stirred solution of 1,5-anhydro-5-thio-D-glucitol (0.194 g, 1.08 mmol) in dry DMF (60 mL) was added NaH (0.5 g, 12.5 mmol) and then BnBr (0.7 mL, 5.9 mmol), and the mixture was stirred overnight. Excess NaH was destroyed by the addition of MeOH. The organic phase was concentrated under reduced pressure. To the residue was added H₂O (200 mL) and this was extracted with CH₂Cl₂ (5×100 mL). The organic phase was dried over Na₂SO₄ and concentrated. The product was purified by flash chromatography [hexanes:EtOAc, 20:1] to give a syrup that was recrystallized from EtOAc/hexanes to give compound 75 as a white solid (0.276 g, 58%). Mp 56-59° C.; [α]_{D}=+15.1 (c 1.1, CHCl₃). The ¹H NMR spectrum was consistent with the literature data.⁷⁹

### 1,5-Anhydro-2,3,4-tri-O-acetyl-5-selenoxylitol (81)

To a stirred suspension of selenium (1.48 g, 18.7 mmol) in anhydrous EtOH (40 mL) at 0° C. was added NaBH₄ (0.9 g, 23.8 mmol). An almost colorless solution resulted. The ice bath was removed and 2,3,5-tri-O-acetyl-1,5-dibromo-1,5-dideoxy-xylitol 80 (4.87 g, 12.0 mmol) was added, and the mixture was stirred at RT overnight. H₂O (200 mL) was added and the mixture was extracted with Et₂O (5×100 mL). The solids were removed by filtration, the solution was concentrated, and the residue was purified by flash chromatography [hexanes:EtOAc, 1:1] to give 81 as yellow crystals (2.22 g, 57%). Mp 106-111° C.; ¹H NMR (CDCl₃): δ 5.11 (2H, ddd, J_{1eq,2}=J_{4,5eq}=4.5 Hz, J_{1ax,2}=J_{4,5ax}=10.8 Hz, H-2, H-4), 4.96 (1H, t, J_{2,3}=J_{3,4}=9.7 Hz, H-3), 2.74 (2H, dd, H-1 eq, H-5eq), 2.67 (2H, t, J_{5ax,5eq}=J_{1ax,1eq}=12.0 Hz, H-1ax, H-5ax), 2.00 (3H, s, OAc), 1.99 (6H, s, OAc); ¹³C NMR (CDCl₃): δ 169.79 and 169.65 (3C═O), 73.98 (C-3), 73.78 (2C, C-2 and C-4), 21.02 (2 OAc), 20.80 (2C, C-1 and C-5), 20.56 (OAc). Anal. Calcd for C₁₁H₁₆O₆Se: C, 40.88; H, 4.99. Found: C, 40.76; H, 5.02.

### 1,5-Anhydro-2,3,4-tri-O-benzyl-5-selenoxylitol (76)

(a) Acetate Methanolysis: A mixture of 1,5-anhydro-2,3,4-tri-O-acetyl-5-selenoxylitol 81 (2.22 g, 6.87 mmol) and 1M NaOMe in MeOH (10 mL, 10 mmol) in dry MeOH (60 mL) was stirred under a N₂ atmosphere overnight. The mixture was netralized with excess Rexyn 101, the resin was removed by filtration, and the organic phase was concentrated to give 1,5-anhydro-selenoxylitol as tan crystals (1.19 g, 88%). Mp 98-105° C.; ¹H NMR (D₂O): δ 3.75 (2H, m, J_{1eq,2}=J_{4,5eq}=4.6 Hz, J_{1ax,2}=J_{4,5ax}=10.8 Hz, H-2, H-4), 3.11 (1H, t, J_{2,3}=J_{3,4}=9.2 Hz, H-3), 2.66 (2H, t, J_{5ax,5eq}=J_{1ax,1eq}=11.8 Hz, H-5ax, H-1ax), 2.60 (2H, dd, H-1 eq, H-5eq); ¹³C NMR (D₂O): δ 81.40 (C-3), 76.62 (2C, C-2 and C-4), 25.65 (2C, C-1 and C-5). Anal. Calcd for C₅H₁₀O₃Se: C, 30.47; H, 5.11. Found: C, 30.29; H, 5.21.

(b) Benzylation: To 1,5-anhydro-5-selenoxylitol 81 (0.289 g, 1.47 mmol) in dry DMF (20 mL) was added 60% NaH (0.516 g, 6 equiv) while stirring in an ice bath. The ice bath was removed and BnBr (0.9 mL, 4 equiv) was added. The mixture was stirred under N₂ overnight. The reaction was then quenched with MeOH(S mL), H₂O (100 mL) was added, and the mixture was extracted with Et₂O (3×50 mL). The organic solution was dried over Na₂SO₄ and concentrated. The product was purified by flash chromatography [hexanes:EtOAc, 20: 1] to give the title compound 76 as a white solid (0.505 g, 74%). Mp 56-60° C.; ¹H NMR (CDCl₃): δ 7.32-7.24 (15H, m, ArH), 4.81 (2H, s, CH₂Ph), 4.70 (2H, d, J_{A,B}=11.6 Hz, CH₂Ph), 4.66 (2H, d, J_{A,B}=11.5 Hz, CH₂Ph), 3.73 (2H, m, J_{1eq,2}=J_{4,5eq}=4.2 Hz, J_{1ax,2}=J_{4,5ax}=11.2 Hz, H-2, H-4), 3.27 (1H, t, J_{2,3}=J_{3,4}=8.9 Hz, H-3), 2.69 (2H, dd, J_{5ax,5eq}=J_{1ax,1eq}=12.0 Hz, H-5eq, H-1eq), 2.58 (2H, t, H-5ax, H-1ax); ³C NMR (CDCl₃): 138.89 (Cᵢₚₛₒ), 138.44 (2Cᵢₚₛₒ), 128.39-127.46 (15C, Ar), 86.98 (C-3), 83.17 (2C, C-2 and C-4), 76.34 (CH₂Ph), 72.97 (2 CH₂Ph), 22.11 (2C, C-1 and C-5). Anal. Calcd for C₂₆H₂₈O₃Se: C, 66.80; H, 6.04. Found: C, 66.88; H, 6.22.

### 1,5-Dideoxy-1,5-[[N-(2R,3R)-2,4-O-benzylidene-2,4-dihydroxy-3-(sulfooxy)-butyl]iminoonium]-xylitol (84)

1,5-Dideoxy-1,5-iminoxylitol 72 (0.161 g, 1.21 mmol) and 2,4-O-benzylidene-D-erythritol-1,3-cyclic sulfate 71b (0.360 g, 1.32 mmol) were dissolved in reagent grade MeOH (2 mL). Anhydrous K₂CO₃ (0.015 g, 0.11 mmol) was added and the mixture was stirred in a sealed tube at 65° C. for 3.5 h, at which point TLC showed that the cyclic sulfate had been consumed. The solvent was removed and the residue was purified by column chromatography (EtOAc:MeOH:H₂O, 8:2:1) to give the product 84 as a yellow oil (0.209 g, 43%): [α]_{D}−50 (c 0.48, H₂O); NMR data in Tables 1 and 3.

### 1,5-Dideoxy-1,5-[[N-(2R,3R)-2,4-dihydroxy-3-(sulfooxy)-butyl]iminoonium]-xylitol (66b)

Aqueous 60% HOAc (25 mL) was added to compound 84 (0.209 g, 0.515 mmol) and the mixture was stirred while warming in an open flask for 20 h at 70° C. The mixture was cooled and concentrated and the crude product was purified by column chromatography (EtOAc:MeOH:H₂O, 6:4:1) to give compound 66b (0.118 g, 72%) as a colorless, hard foam: [α]_{D}−9 (c 0.57, H₂O); NMR data in Tables 2 and 4; MALDI MS m/e 339.99 (M⁺+Na), 238.12 (M⁺+H−SO₃).

### 1,5-Dideoxy-1,5-[[N-(2S,3S)-2,4-O-benzylidene-2,4-dihydroxy-3-(sulfooxy)-butyl]iminoonium]-xylitol (82)

1,5-Didexy-1,5-iminoxylitol 72 (0.158 g, 1.19 mmol) and 2,4-O-benzylidene-L-erythritol-1,3-cyclic sulfate 71a (0.347 g, 1.27 mmol) were dissolved in reagent grade MeOH (2 mL). Anhydrous K₂CO₃ (0.018 g, 0.15 mmol) was added and the mixture was stirred in a sealed tube at 65° C. for 4 h. The solvent was removed and the residue as purified by column chromatography (EtOAc:MeOH:H₂O, 8:2:1) to give the product 82 as a yellow oil (0.273 g, 56%). [α]_{D}+55 (c 0.65, H₂O); ¹H and ¹³C NMR data were virtually identical with those of the enantiomer 84 (see Tables 1 and 3); MALDI MS m/e 428.09 (M⁺+Na), 406.11 (M⁺+H), 326.15 (M⁺+H−SO₃).

### 1,5-Dideoxy-1,5-[[N-(2S,3S)-2,4-dihydroxy-3-(sulfooxy)-butyl]iminoonium]-xylitol (66a)

Aqueous 60% HOAc (25 mL) was added to compound 82 (0.273 g, 0.673 mmol) and the mixture was stirred while warming in an open flask for 14 h at 75° C. The mixture was cooled and concentrated and the crude product was purified by column chromatography (EtOAc:MeOH:H₂O, 6:4:1) to give compound 66a (0.156 g, 73%) as a colorless, hard foam. [α]_{D}+11 (c 0.56, H₂O);¹H and ¹³C NMR data were virtually identical to those of the enantiomer 66b (see Tables 2 and 4); MALDI MS m/e 399.99 (M⁺+Na), 318.28 (M⁺+H), 238.12 (M⁺+H−SO₃).

### 2,3,4,6-Tetra-O-benzyl-1,5-dideoxy-1,5-[[N-(2R,3R)-2,4-O-benzylidene-2,4-dihydroxy-3-(sulfooxy)-butyl]iminoonium]-D-glucitol (86)

Tri-O-benzyldeoxynojirimycin 73 (0.241 g, 0.460 mmol) and 2,4-O-benzylidene-D-erythritol-1,3-cyclic sulfate 71b (0.143 g, 0.525 mmol) were dissolved in reagent grade acetone (2 mL). Anhydrous K₂CO₃ (0.020 g, 0.15 mmol) was added and the mixture was stirred in a sealed tube at 70° C. for 20 h. The solvent was removed and the residue was purified by column chromatography (CHCl₃: MeOH, 5:1) to give the product 86 as a colorless gum (0.240 g, 65%). [α]_{D}−5.4 (c 0.9, CHCl₃); NMR data in Tables 1 and 3.

### 1,5-Dideoxy-1,5-[[N-(2R,RS)-2,4-dihydroxy-3-(sulfooxy)-butyl]iminoonium]-D-glucitol (67b)

Compound 86 (0.209 g, 0.263 mmol) was dissolved in 80% aqueous acetic acid (20 mL) and the solution was stirred with 10% Pd/C catalyst (0.42 g) under 1 atm of H₂ for 20 h. The catalyst was removed by filtration through a small plug of silica gel, and washed with water (50 mL). The filtrate was evaporated and the gummy residue was freed of acetic acid by dissolving in water and re-concentrating (2×50 mL). The crude product was purified by column chromatography (EtOAc: MeOH: H₂O, 6:3:1) to give compound 67b (0.096 g, containing 0.56 equiv. or 13% by weight of KOAc by ¹H NMR, 91% after correcting for acetate content). NMR data in Tables 2 and 4.

2,3,4,6-Tetra-O-benzyl-1,5-dideoxy-1,5-[[N-(2S,3S)-2,4-O-benzylidene-2,4-dihydroxy-3-(sulfooxy)-butyl]iminoonium]-D-glucitol (85).

Tri-O-benzyldeoxynojirimycin 73 (0.223 g, 0.426 mmol) and 2,4-O-benzylidene-L-erythritol-1,3-cyclic sulfate 71a (0.123 g, 0.4535 mmol) were dissolved in reagent grade acetone (2 mL). Anhydrous K₂CO₃ (0.020 g, 0.15 mmol) was added and the mixture was stirred in a sealed tube at 70° C. for 20 h. The solvent was removed and the residue was purified by column chromatography (CHCl₃: MeOH, 5:1) to give the product 85 as a colorless amorphous solid (0.271 g, 80%): [α]_{D}+36 (c 0.8, CHCl₃); NMR data in Tables 1 and 3.

### 1,5-Dideoxy-1,5-[[N-(2R,3R)-2,4-dihydroxy-3-(sulfooxy)-butyl]iminoonium]-D-glucitol (67a)

Compound 85 (0.205 g, 0.263 mmol) was dissolved in 80% aqueous acetic acid (20 mL) and the solution was stirred with 10% Pd/C catalyst (0.41 g) under 1 atm of H₂ for 20 h. The catalyst was removed by filtration through a small plug of silica gel, and washed with water (50 mL). The filtrate was evaporated and the gummy residue was freed of acetic acid by dissolving in water and re-concentrating (2×50 mL). The crude product was purified by column chromatography (EtOAc: MeOH: H₂O, 6:3:1) to give compound 67a (0.094 g, containing 0.77 equiv. or 18% by weight of KOAc by ¹H NMR, 89% after correcting for acetate content). See Tables 2 and 4 for ¹H and ¹³C NMR data.

### 2,3,4-Tri-O-benzyl-1,5-dideoxy-1,5-[[(2R,3R)-2,4-O-benzylidene-2,4-dihydroxy-3-(sulfooxy)butyl]-(S)-episulfoniumylidene]-xylitol inner salt (88b) and 2,3,4-tri-O-benzyl-1,5-dideoxy-1,5-[[(2R,3R)-2,4-O-benzylidene-2,4-dihydroxy-3-(sulfooxy) butyl]-(R)-episulfoniumylidene]-xylitol Inner Salt (89b)

To 1,1,1,3,3,3-hexafluoro-2-propanol (0.5 mL) were added 2,4-O-benzylidene-D-erythritol-1,3-cyclic-sulfate 71b (0.565 g, 2.08 mmol), 1,5-anhydro-2,3,4-tri-O-benzyl-5-thioxylitol 7 (0.677 g, 1.61 mmol) and anhydrous K₂CO₃ (70 mg). The mixture was stirred in a sealed tube in a 70° C. oil bath overnight, after which an extra 40 mg of anhydrous K₂CO₃ was added. The solvents were removed and the residue was chromatographed [CHCl₃:MeOH, 10:1] to give 88b and 89b in a 2:1 ratio (0.975 g, 87%).

Major isomer 88b: mp 186-189° C.; [α]_{D}+2.1 (c 1.2, CH₂Cl₂); NMR data in Tables 1 and 3; HRMS Calcd for C₃₇H₄₀O₉S₂ (M+H): 693.2192. Found: 693.2209. Anal. Calcd for C₃₇H₄₀O₉S₂: C, 64.14; H, 5.82. Found: C, 64.39; H, 5.94.

Minor isomer 89b: mp 169-172° C.; [α]_{D}−49.1 (c 0.8, CH₂Cl₂); NMR data in Tables 1 and 3; Anal. Calcd for C₃₇H₄₀O₉S₂: C, 64.14; H, 5.82. Found: C, 63.84: H, 5.96.

### 1,5-Dideoxy-1,5-[[(2R,3R)-2,4-dihydroxy-3-(sulfooxy)butyl]-(S)-episulfoniumylidene]-xylitol Inner Salt (S-68b)

To compound 88b (0.33 g, 0.48 mmol) dissolved in 80% AcOH (12 mL) was added Pd(OH)₂ (0.2 g). The mixture was stirred under H₂ (110 psi) for 48 h and then filtered through Celite with MeOH. The solvent was evaporated and the residue was purified by column chromatography [EtOAc:MeOH:H₂O, 7:3:1]. Compound S-68b was obtained as a syrup (0.13 g, 81%); [α]_{D}−21.8 (c 1.1, H₂O); NMR data in Tables 2 and 4; HRMS Calcd for C₉H₁₉O₉S₂ (M+H): 335.0470. Found: 335.0454. Anal. Calcd for C₉H₁₈O₉S₂: C, 32.33; H, 5.43. Found: C, 32.03; H, 5.59.

### 1,5-Dideoxy-1,5-[[(2R,3R)-2,4-dihydroxy-3-(sulfooxy)butyl]-(R)-episulfoniumylidene]-xylitol Inner Salt (R-68b)

Compound 89b (0.249 g, 0.36 mmol) was deprotected by hydrogenolysis using the procedure described above for S-68b to give the title compound as a syrup (0.13 g, 95%); [α]_{D}−16.2 (c 0.9, H₂O); NMR data in Tables 2 and 4; HRMS Calcd for C₉H₁₉O₉S₂ (M+H): 335.0470. Found: 335.0478. Anal. Calcd for C₉H₁₈O₉S₂: C, 32.33; H, 5.43. Found: C, 31.88; H, 5.21.

### 2,3,4-Tri-O-benzyl-1,S-dideoxy-1,5-[[(2S,3S)-2,4-O-benzylidene-2,4-dihydroxy-3-(sulfooxy) butyl]-(R)-episulfoniumylidene]-xylitol Inner Salt (88a) and 2,3,4-tri-O-benzyl-1,5-dideoxy-1,5-[[(2S,3S)-2,4-O-benzylidene-2,4-dihydroxy-3-(sulfooxy) butyl]-(S)-episulfoniumylidene]-xylitol Inner Salt (89a)

To 1,1,1,3,3,3-hexafluoro-2-propanol (0.5 mL) were added 2,4-O-benzylidene-L-erythritol-1,3-cyclic-sulfate 71a (0.265 g, 0.97 mmol), 1,5-anhydro-2,3,4-tri-O-benzyl-5-thioxylitol 74 (0.328 g, 0.78 mmol) and anhydrous K₂CO₃ (24 mg). The mixture was stirred in a sealed tube in a 70° C. oil bath for 5 days. The solvent was evaporated and the residue was purified by column chromatography [CHCl₃:MeOH, 10:1] to give 88a and 89a in a 5:2 ratio as a white solid (0.465 g, 86%). Pure samples were obtained by rechromatography.

Major isomer 88a: Mp 175-180° C.; [α]_{D}−3.7 (c 0.9, CH₂Cl₂); ¹H and ¹³C NMR data were virtually identical to those of the enantiomer 88b. Anal. Calcd for C₃₇H₄₀O₉S₂: C, 64.14; H, 5.82; Found: C, 63.81; H, 5.68.

Minor isomer 89a: Mp 163-170° C.; [α]_{D}+41.8 (c 1.1, CH₂Cl₂); ¹H and ¹³C NMR data were virtually identical to those of the enantiomer 89b. Anal. Calcd for C₃₇H₄₀O₉S₂: C, 64.14; H, 5.82. Found: C, 64.42; H, 5.75.

### 1,5-Dideoxy-1,5-[[(2S,3S)-2,4-dihydroxy-3-(sulfooxy)butyl]-(R)-episulfoniumylidene]-xylitol Inner Salt (R-68a)

To compound 88a (0.304 g, 0.44 mmol) dissolved in 80% AcOH (10 mL) was added Pd/C (0.5 g). The mixture was stirred under 120 psi H₂ for 96 h. The mixture was filtered through Celite with MeOH, and the solvent removed. The residue was then redissolved in 80% AcOH (10 ml). To the solution was added Pd(OH)₂ (0.2 g) and the solution was stirred under 120 psi H₂ for 48 h. The mixture was filtered through Celite with MeOH, the solvent evaporated, and the residue was purified by column chromatography [EtOAc:MeOH:H₂O, 7:3:1] to give the title compound as a syrup (0.08 g, 55%); [α]_{D}+21.7 (c 0.8, H₂O). ¹H and ¹³C NMR data were virtually identical to those of the enantiomer S-68b (see Tables 1 and 3). HRMS Calcd for C₉H₁₈O₉S₂Na (M+Na): 357.0290. Found: 357.0284.

### 1,5-Dideoxy-1,5-[[(2S,3S)-2,4-dihydroxy-3-(sulfooxy)butyl]-(S)-episulfoniumylidene]-xylitol Inner Salt (S-68a)

Compound 89a (0.240 g, 0.35 mmol) was deprotected by hydrogenolysis using the procedure described above for S-68b to give the title compound as a syrup (0.08 g, 67%); [α]_{D}+19.5 (c 0.7, H₂O). ¹H and ¹³C NMR data were virtually identical to the enantiomer R-68b (see Tables 2 and 4) HRMS Calcd for C₉H₁₉O₉S₂ (M+H): 335.0470. Found: 335.0477.

2,3,4,6-Tetra-O-benzyl-1,5-dideoxy-1,5-[[(2R,3R)-2,4-O-benzylidene-2,4-dihydroxy-3-(sulfooxy) butyl]-(S/R)-episulfoniumylidene]-D-glucitol inner salts (90b) and (91b).

To 1,1,1,3,3,3-hexafluoro-2-propanol (0.5 mL) were added 2,4-O-benzylidene-D-erythritol-1,3-cyclic-sulfate 71b (0.115 g, 0.42 mmol), 1,5-anhydro-2,3,4,6-tetra-O-benzyl-5-thio-D-glucitol 75 (0.174 g, 0.32 mmol) and anhydrous K₂CO₃ (30 mg). The mixture was stirred in a sealed tube in a 70° C. oil bath for 5 days. The solvent was removed and the residue was purified by column chromatography [CHCl₃:MeOH, 10:1] to give an inseparable mixture of 90b and 91b in a 2:1 ratio as a white solid (0.182 g, 70%); [α]_{D}=+2.1 (c 1.3, CH₂Cl₂). Major isomer 90b: See Tables 1 and 2 for ¹H and ¹³C NMR data. Anal. Calcd for C₄₅H₄₈O₁₀S₂: C, 66.48; H, 5.96. Found: C, 66.36; H, 6.08.

### 1,5-Dideoxy-1,5-[[(2R,3R)-2,4-dihydroxy-3-(sulfooxy)butyl]-(S)-episulfoniumylidene]-D-glucitol Inner Salt (69b)

To a mixture of compounds 90b and 91b (0.1639 g, 0.20 mmol) dissolved in 80% AcOH (10 mL) was added Pd(OH)₂ (0.17 g). The mixture was stirred under 120 psi H₂ for 48 h. The mixture was filtered through Celite with MeOH, the solvent was removed, and the residue was purified by column chromatography [EtOAc:MeOH:H₂O, 7:3:1]. Compound 69b was obtained as a syrup (0.06 g, 81%); [α]_{D}=−20.4 (c 0.8, H₂O). See Tables 2 and 4 for ¹H and ¹³C NMR data. HRMS. Calcd for C₁₀H₂₁O₁₀S₂ (M+H): 365.0576 Found: 365.0574.

### 2,3,4,6-Tetra-O-benzyl-1,5-dideoxy-1,5-[[(2S,3S)-2,4-O-benzylidene-2,4-dihydroxy-3-(sulfooxy) butyl]-(R/S)-episulfoniumylidene]-D-glucitol Inner Salts (90a) and (91a)

To 1,1,1,3,3,3-hexafluoro-2-propanol (0.5 mL) were added 2,4-O-benzylidene-L-erythritol-1,3-cyclic-sufate 71a (0.148 g, 0.54 mmol), 1,5-anhydro-2,3,4,6-tetra-0-benzyl-5-thio-D-glucitol 75 (0.240 g, 0.44 mmol) and anhydrous K₂CO₃ (33 mg). The mixture was stirred in a sealed tube in a 69-70° C. oil bath for 84 h. The solvent was evaporated and the residue was purified by column chromatography [CHCl₃:MeOH, 10:1] to give an inseparable 3:1 mixture of 90a and 91a as a white solid (0.25 g, 68%); [α]_{D}=+48.8 (c 1.6, CH₂Cl₂). Major isomer 90a: See Tables 1 and 2 for ¹H and ¹³C NMR data. Anal. Calcd for C₄₅H₄₈O₁₀S₂: C, 66.48; H, 5.95. Found: C, 66.19; H, 6.07.

### 1,5-Dideoxy-1,5-[[(2S,3S)-2,4-dihydroxy-3-(sulfooxy)butyl]-(R)-episulfoniumylidene]-D-glucitol Inner Salt (69a)

To a mixture of compounds 90a and 91a (0.180 g, 0.22 mmol) dissolved in 80% AcOH (10 mL) was added Pd(OH)₂ (0.20 g), and the mixture was stirred under 120 psi H₂ for 6 days. The mixture was filtered through Celite with MeOH, the solvent was removed and the residue was purified by column chromatography [EtOAc:MeOH:H₂O, 7:3:1]. Compound 69a was obtained as a syrup (0.05 g, 67%); [α]_{D}=+10.3 (c 0.6, H₂O). See Tables 2 and 4 for ¹H and ¹³C NMR data. HRMS Calcd for C₁₀H₂₁O₁₀S₂ (M+H): 365.0576. Found: 365.0577.

### 2,3,4-Tri-O-benzyl-1,5-dideoxy-1,5-[[(2R,3R)-2,4-O-benzylidene-2,4-dihydroxy-3-(sulfooxy)butyl]-(S/R)-episelenoniumylidene]-xylitol Inner Salt (92b and 93b)

To 1,1,1,3,3,3-hexafluoro-2-propanol (0.5 mL) were added 2,4-O-benzylidene-D-erythritol-1,3-cyclic-sufate 71b (0.272 g, 1.00 mmol), 1,5-anhydro-2,3,4-tri-O-benzyl-5-selenoxylitol 76 (0.362 g, 0.78 mmol) and anhydrous K₂CO₃ (50 mg). The mixture was stirred in a sealed tube in a 70° C. oil bath for 48 h. The solvent was concentrated and the residue was purified by column chromatography [CHCl₃:MeOH, 10:1] to give an inseparable mixture of 92b and 93b in a 1:4 ratio (0.20 g, 96%). [α]_{D}−45.7 (c 1.1, CH₂Cl₂). For the major isomer 36b: See Tables 1 and 2 for ¹H and ¹³C NMR data. Anal. Calcd for C₃₇H₄₀O₉SSe: C, 59.99; H, 5.45. Found: C, 59.73; H, 5.36.

### 1,5-Dideoxy-1,5-[[(2R,3R)-2,4-dihydroxy-3-(sulfooxy)butyl]-(R)-episelenoniumylidene]-xylitol Inner Salt (70b)

To the mixture of compounds 92b and 93b (0.295 g, 0.40 mmol) dissolved in 80% AcOH (10 mL) was added Pd(OH)₂ (0.29 g), and the mixture was stirred under 120 psi H₂ for 5 days. TLC revealed one major product and two minor products. The mixture was filtered through Celite, concentrated, and the residue was purified by column chromatography [EtOAc:MeOH:H₂O, 7:3:1] to give the major product, compound 70b as a syrup (0.06 g, 39%); [α]_{D}−16.6 (c 0.9, H₂O). See Tables 2 and 4 for ¹H and ¹³C NMR data. HRMS Calcd for C₉H₁₉O₉SSe (M+H): 382.9915. Found: 382.9916. Anal. Calcd for C₉H₁₈O₉SSe: C, 28.35; H, 4.76. Found: C, 28.44; H, 4.71.

### 2,3,4-Tri-O-benzyl-1,5-dideoxy-1,5-[[(2S,3S)-2,4-O-benzylidene-2,4-dihydroxy-3-(sulfooxy)butyl]-(R/S)-episelenoniumylidene]-xylitol Inner Salts (92a and 93a)

To 1,1,1,3,3,3-hexafluoro-2-propanol (0.5 mL) were added 2,4-O-benzylidene-L-erythritol-1,3-cyclic-sufate 71a (0.226 g, 0.83 mmol), 1,5-anhydro-2,3,4-tri-O-benzyl-5-selenoxylitol 76 (0.308 g, 0.66 mmol) and anhydrous K₂CO₃ (20 mg). The mixture was stirred in a sealed tube in a 70° C. oil bath for 72 h. The solvent was removed and the residue was purified by column chromatography [CHCl₃:MeOH, 10:1] to give an inseparable 1:3 mixture of 92a and 93a as a white solid (0.42 g, 85%). [α]_{D}−44.0 (c 0.9, CH₂Cl₂). For the major isomer 93a, the ¹H and ¹³C NMR data were virtually identical to those of the enantiomer (compound 93b, see Tables 1 and 2) except for small chemical shift differences due to concentation effects. Anal. Calcd for C₃₇H₄₀O₉SSe: C, 59.99; H, 5.45. Found: C, 59.85; H, 5.58.

### 1,5-Dideoxy-1,5-[[(2S,3S)-2,4-dihydroxy-3-(sulfooxy)butyl]-(S)-episelenoniumylidene]-xylitol Inner Salt (70a)

To a mixture of compounds 92a and 93a (0.406 g, 0.55 mmol) dissolved in 80% AcOH (10 mL) was added Pd(OH)₂ (0.50 g), and the mixture was stirred under 120 psi H₂ for 8 days. TLC revealed one major product and two minor products. The mixture was filtered through Celite with MeOH, the solvent was removed, and the residue was purified by column chromatography [EtOAc/MeOH/H₂O, 7:3:1]. Compound 70a was obtained as a syrup (0.05 g, 25%); [α]_{D}+14.1 (c 0.4, H₂O). For compound 70a, the ¹H and ¹³C NMR data were virtually identical to the enantiomer (compound 70b, see Tables 1 and 2) except for small chemical shift differences due to concentration effects. HRMS Calcd for C₉H₁₈O₉SSeNa (M+Na): 404.9734. Found: 404.9735. Anal. Calcd for C₉H₁₈O₉SSe: C, 28.35; H, 4.76. Found: C, 28.56; H. 4.54.

TABLE 1
¹H NMR Data for Compounds 84, 85, 86, 88b, 89b, 90a, 90b and 93b.

| Compound | 84^{a}* | 85^{b} | 86^{c} | 88b^{d}* | 89b^{e}* | 90a^{f} | 90b^{g} | 93b^{h}* |
|---|---|---|---|---|---|---|---|---|
| H-1eq | 3.18-3.13 | 3.47(dd) | 3.33(dd) | 3.59(ddd) | 3.38(d) | 3.82(dd) | 3.40(dd) | 3.36(dd) |
| (J_{1eq,2,} J_{1eq,5eq}) | (m) | (4.9, ˜0) | (4.8, ˜0) | (4.4, 2.4) | (2.9, ˜0) | (4.5, ˜01) | (4.5, ˜0) | (3.2, ˜0) |
| | (nd, nd) | | | | | | | |
| H-1ax | 2.32(dd) | 2.34(dd) | 2.58(dd) | 3.38(dd) | 3.38(d) | 3.57(dd) | 3.31(dd) | 3.18(dd) |
| (J_{1ax,1eq,} J_{1ax,2}) | (9.1, 9.1) | (11.5, 11.0) | (11.1, 10.6) | (12.5, 2.1) | (nd, 2.9) | (14.9, 2.7) | (14.7, 3.2) | (13.3, 4.0) |
| H-2(J_{2,3,} J_{2,4}) | 3.61-3.53 | 3.62(ddd) | 3.58(ddd) | 3.89(dddd) | 3.84(ddd) | 4.20(ddd) | 3.92(ddd) | 3.86(ddd) |
| | (m) | (9.0, ˜0) | (9.0, ˜0) | (3.6, 1.8) | (˜2, ˜0) | (4.3, ˜0) | (4.5, ˜0) | (4.2, ˜0) |
| | (10.9, nd) | | | | | | | |
| H-3(J_{3,4}) | 3.22(dd) | 3.38 dd) | 3.39 dd) | 3.79(dd) | 3.92(dd) | 3.87(dd) | 3.85(dd) | 3.91(dd) |
| | (10.9) | (9.0) | (9.0) | (3.6) | (<1) | (4.3) | (4.5) | (4.2) |
| H-4(J_{4,5eq,} J_{4,5ax}) | 3.61-3.53 | 3.55(ddd) | 3.54(dd) | 4.05(dddd) | 4.02(ddd) | 3.79(dd) | 4.02(dd) | 3.98(ddd) |
| | (m) | (na, 9.5) | (na, 9.5) | (4.1, 1.9) | (3.5, 2.6) | (4.3, na) | (5.1, na) | (4.5, 2.9) |
| | (nd, 9.1) | | | | | | | |
| H-5eq | 3.18-3.13 | na | na | 3.93(ddd) | 3.76(dd) | 3.90(ddd) | 4.09(ddd) | 3.56(ddd) |
| | (m) | | | | | | | |
| H-5ax(J_{5eq,5ax}) | 2.32(dd) | 2.40(ddd) | 2.55(ddd) | 3.54(dd) | 3.64(dd) | na | na | 3.66(dd) |
| | (9.1) | (na) | (na) | (12.6) | (15.1) | | | (13.4) |
| H-6a(J_{6a,6b}, J_{5,6a}) | na | 3.90(dd) | 3.85(dd) | na | na | 3.72(dd) | 4.03(dd) | na |
| | | (10.7, 2.2) | (10.2, 2.4) | | | (11.1, 7.3) | (10.5, 5.7) | |
| H-6b(J_{5,6b}) | na | 3.68(dd) | 3.78(dd) | na | na | 3.63(dd) | 3.95(dd) | na |
| | | (1.6) | (2.0) | | | (5.1) | (5.3) | |
| H-1′a(J_{1′a,1′b}, J_{1′a,2′}) | 3.12(d) | 3.56(d) | 3.67(d) | 4.28(dd) | 4.99(dd) | 4.67(dd) | 4.70(dd) | 4.82(dd) |
| | (14.2, <1) | (15.1, <1) | (15.6, <1) | (13.5, 3.1) | (14.2, 3.7) | (13.9, 3.7) | (13.9, 4.1) | (12.8, 4.0) |
| H-1′b(J_{1′b,2′}) | 2.83(dd) | 2.78(dd) | 2.93(dd) | 3.67(dd) | 4.27(dd) | 4.32(dd) | 4.32(dd) | 4.30(dd) |
| | (8.3) | (8.0) | (7.9) | (3.9) | (1.5) | (1.8) | (1.9) | (1.7) |
| H-2′(J_{2′,3′}) | 4.14(dd) | 4.05(dd) | 4.07(dd) | 4.32(ddd) | 4.21(ddd) | 4.21(ddd) | 4.22(ddd) | 4.18(ddd) |
| | (9.7) | (10.0) | (9.7) | (10.0) | (10.1) | (9.7) | (9.6) | (9.6) |
| H-3′(J_{3′,4′eq}) | 4.21(ddd) | 4.11(ddd) | 4.16(ddd) | 4.62(ddd) | 4.66(ddd) | 4.66(ddd) | 4.62(ddd) | 4.54(ddd) |
| | (5.2) | (5.4) | (5.3) | (5.4) | (5.5) | (5.5) | (5.5) | (5.3) |
| H-4′eq(J_{4′eq,4′ax}) | 4.50(dd) | 4.55(dd) | 4.60(dd) | 4.49(dd) | 4.47(dd) | 4.50(dd) | 4.51(dd) | 4.45(dd) |
| | (11.0) | (10.6) | (11.0) | (10.9) | (10.8) | (10.5) | (10.7) | (10.6) |
| H-4′ax(J_{3′,4′ax}) | 3.89(dd) | 3.77(dd) | 3.82(dd) | 3.77(dd) | 3.74(dd) | 3.74(dd) | 3.74(dd) | 3.76(dd) |
| | (9.8) | (9.8) | (9.7) | (10.3) | (10.1) | (10.9) | (10.2) | (10.1) |

Footnotes for Table 1
^{a}500MHz, pH=8, D₂O. Others: 7.51-7.43(5H, m, Ar), 5.72(1H, s, benzylidene CH).
^{b}500MHz, pH=10, CD₃OD. Others: 7.44-7.03(25H, m, Ar), 5.55(1H, s, benzylidene CH), 4.89 and 4.73(2H, 2d, J_{A,B}=11.2Hz, CH₂Ar), 4.75 and 4.41(2H, 2d, J_{A,B}=11.0Hz, CH₂Ar), 4.62 and 4.55(2H, 2d, J_{A,B}=11.5Hz, CH₂Ar), 4.57 and 4.43(2H, 2d, J_{A,B}=12.2Hz, CH₂Ar).
^{c}500MHz, pH=10, CD₃OD. Others: 7.50-7.00(25H, m, Ar), 5.61(1H, s, benzylidene CH), 4.88 and 4.74(2H, 2d, J_{A,B}=11.3Hz, CH₂Ar), 4.74 and 4.38(2H, 2d, J_{A,B}=10.8Hz, CH₂Ar), 4.70 and 4.46(2H, 2d, J_{A,B}=11.7Hz, CH₂Ar), 4.62 and 4.57(2H, 2d, J_{A,B}=11.9Hz, CH₂Ar).
^{d}600MHz, CD₂Cl₂. Others: 7.45-7.10(20H, m, Ar), 5.55(1H, s, benzylidene CH), 4.69 and 4.49(2H, 2d, J_{A,B}=11.5Hz, CH₂Ar), 4.49 and 4.43(2H, 2d, J_{A,B}=11.8Hz, CH₂Ar), 4.46 and 4.44(2H, 2d, J_{A,B}=11.6Hz, CH₂Ar).
^{e}600MHz, CD₂Cl₂. Others: 7.45-7.05(20H, m, Ar), 5.52(1H, s, benzylidene CH), 4.64 and 4.58(2H, 2d, J_{A,B}=11.4Hz, CH₂Ar), 4.49 and 4.46(2H, 2d, J_{A,B}=11.9Hz, CH₂Ar), 4.40(2H, s, CH₂Ar).
^{f}500MHz, CD₂Cl₂. Others: 7.46-7.01(25H, m, Ar), 5.52(1H, s, benzylidene CH), 4.65 and 4.54(2H, 2d, J_{A,B}=11.5Hz, CH_{2.}Ar), 446 and 4.41(2H, 2d, J_{A,B}=11.7Hz, CH₂Ar), 4.46 and 4.43(2H, 2d, J_{A,B}=11.4Hz, CH₂Ar), 4.32 and 4.29(2H, 2d, J_{A,B}=11.9Hz, CH₂Ar).
^{g}400MHz, CD₂Cl₂. Others: 7.44-7.06(25H, m, Ar), 5.52(1H, s, benzylidene CH), 4.66 and 4.50(2H, 2d, J_{A,B}=11.6Hz, CH_{2.}Ar), 4.61 and 4.55(2H, 2d, J_{A,B}=11.4Hz, CH₂Ar), 4.48 and 4.44(2H, 2d, J_{A,B}=11.7Hz, CH₂Ar), 4.40(2H, s, CH₂Ar).
^{h}600MHz, CD₂Cl₂. Others: 7.40-7.10(20H, m, Ar), 5.55(1H, s, benzylidene CH), 4.63 and 4.57(2H, 2d, J_{A,B}=11.4Hz, CH₂Ar), 4.50 and 4.47(2H, 2d, J_{A,B}=11.8Hz, CH₂Ar), 4.47 and 4.42(2H, 2d, J_{A,B}=11.8Hz, CH₂Ar).
*Assignments for diastereotopic H-1/H-5 and H-2/H-4 pairs may be reversed.
na = not applicable,
n.d. = not determined

TABLE 2
¹³C NMR Data for Compounds 84, 85, 86, 88b, 89b, 90a, 90b and 93b.

| Compound | 84^{a}* | 85^{b} | 86^{c} | 88b^{d}* | 89b^{e}* | 90a^{f} | 90b^{g} | 93b^{h}* |
|---|---|---|---|---|---|---|---|---|
| C-1 | 57.89 | 56.62 | 56.96 | 42.08 | 32.90 | 32.49 | 34.69 | 30.17 |
| C-2 | 69.92 | 79.27 | 79.48 | 71.61 | 70.99 | 72.52 | 73.49 | 72.16 |
| C-3 | 78.72 | 88.10 | 88.17 | 70.94 | 70.31 | 73.84 | 76.87 | 72.48 |
| C-4 | 69.92 | 79.71 | 79.70 | 71.72 | 70.72 | 73.16 | 73.66 | 72.08 |
| C-5 | 58.07 | 66.55 | 64.35 | 39.24 | 33.41 | 52.36 | 53.46 | 30.73 |
| C-6 | na | 66.16 | 66.05 | na | na | 65.89 | 65.33 | na |
| C-1′ | 57.77 | 54.19 | 53.95 | 46.00 | 43.31 | 44.94 | 45.38 | 43.37 |
| C-2′ | 77.78 | 79.81 | 77.28 | 76.75 | 77.44 | 77.10 | 77.23 | 77.47 |
| C-3′ | 69.18 | 69.53 | 69.34 | 66.88 | 65.73 | 65.80 | 66.63 | 67.41 |
| C-4′ | 69.06 | 70.56 | 70.61 | 69.51 | 69.51 | 69.50 | 69.59 | 69.51 |

Footnotes for Table 2
^{a}125MHz, D₂O. Others: 136.85(Cᵢₚₛₒ, Ar), 130.34, 129.28, 126.66(5C, Ar), 101.44(benzylidene CH)
^{b}125MHz, CD₃OD. Others: 140.32, 139.82, 139.76and 139.36(2C)(5 × Cᵢₚₛₒ, Ar), 129.93-127.27(25C, Ar), 101.97(benzylidene CH), 76.19(2C), 74.34 and 73.23(4 × CH₂Ar).
^{c}125MHz, CD₃OD. Others: 140.35, 139.88, 139.71, 139.506 and 139.41(5 × Cᵢₚₛₒ, Ar), 130.12-127.27(25C, Ar), 101.69(benzylidene CH), 76.23(2C), 74.25 and 73.34(4 × CH₂Ar).
^{d}100MHz, CD₂Cl₂. Others: 137.35, 136.96, 136.92 and 136.85(4 × Cᵢₚₛₒ, Ar), 128.84-126.54(20C, Ar), 102.07(benzylidene CH), 73.68, 72.17 and 72.00(3 × CH₂Ar).
^{e}100MHz, CD₂Cl₂. Others: 137.38, 137.11, 137.00 and 136.80(4 × Cᵢₚₛₒ, Ar), 129.80-126.48(20C, Ar), 102.19(benzylidene CH), 73.59, 72.64 and 72.10(3 × CH₂Ar).
^{f}100MHz, CD₂Cl₂. Others: 137.18, 137.07, 137.00, 136.85 and 136.75(5 × Cᵢₚₛₒ, Ar), 129.71-126.65(25C, Ar), 102.11(benzylidene CH), 73.70, 73.51, 73.40 and 71.85(4 × CH₂Ar).
^{g}100MHz, CD₂Cl₂. Others: 137.54, 137.44, 137.35, 137.17 and 136.85(5 × Cᵢₚₛₒ, Ar), 129.80-126.54(25C, Ar), 101.95(benzylidene CH), 74.48, 74.13, 73.99 and 72.35(4 × CH₂Ar).
^{h}100MHz, CD₂Cl₂. Others: 137.39, 137.29, 137.13 and 137.09(4 × Cᵢₚₛₒ, Ar), 129.74-126.49(25C, Ar), 102.04(benzylidene CH), 73.37, 72.83 and 72.255(3 × CH₂Ar).
*Assignments for diastereotopic C-1/C-5 and C-2/C-4 pairs may be reversed.

TABLE 3
¹H NMR Data Compounds 66b, 67a, 67b, S-68b, R-68b, 69a, 69b and 70b.

| Compound | 66b^{a}* | 67a^{b} | 67b^{c} | S-68b^{d}* | R-68b^{e}* | 69a^{f} | 69b^{g} | 70b^{h}* |
|---|---|---|---|---|---|---|---|---|
| H-1eq | 3.08 dd) | 3.57(br d) | 3.16(dd) | 3.70(dd) | 3.38-3.72(m) | 3.85(dd) | 3.89(dd) | 3.36(dd) |
| (J_{1eq,2}) | (4.8) | (4.8) | (4.9) | (3.5) | (3.8) | (3.6) | (3.9) | (3.2, <1) |
| H-1ax | 2.18(dd) | 2.93(br dd) | 2.49(dd) | 3.54(dd) | 3.36(d) | 3.52(dd) | 3.45(dd) | 3.18(dd) |
| (J_{1ax,1eq,} | (11.0, 11.0) | (11.8, 10.8) | (11.7, 11.1) | (13.9, 7.2) | (11.8, 11.8) | (11.5, 11.5) | (11.5, 11.5) | (13.3, 4.0) |
| J_{1ax,2}) | | | | | | | | |
| H-2 | 3.58(ddd) | 3.80(ddd) | 3.55(ddd) | 4.29(ddd) | 4.01-3.93(m) | 3.91(ddd) | 3.97(ddd) | 3.86(ddd) |
| (J_{2,3}) | (9.2) | (9.4) | (9.3) | (7.2) | (9.0) | (8.5) | (9.1) | (4.2, <1) |
| H-3 | 3.20(dd) | 3.47 dd) | 3.30 dd) | 3.75(dd) | 3.51(dd) | 3.51(dd) | 3.54(dd) | 3.91(dd) |
| (J_{3,4}) | (9.2) | (9.4) | (9.5) | (7.2) | (9.0) | (8.5) | (9.1) | (4.2) |
| H-4 | 3.55(ddd) | 3.62(dd) | 3.44(dd) | 4.28(ddd) | 4.01-3.93(m) | 3.80(dd) | 3.86(dd) | 3.98(ddd) |
| (J_{4,5eq,} J_{4,5ax}) | (4.7, 11.0) | (na, 9.8) | (na, 9.5) | (3.5, 7.2) | (3.5, 11.8) | (na, 10.5) | (na, 10.8) | (2.9, 4.5) |
| H-5eq | 3.073(dd) | na | na | 3.71(ddd) | 3.38-3.72(m) | na | na | 3.66(ddd) |
| (J_{5eq,6a}) | na | | | (na) | (na) | | | (na) |
| H-5ax | 2.13(dd) | 3.07(br m) | 2.37(ddd) | 3.55(dd) | 3.36(dd) | 3.75(ddd) | 3.76(ddd) | 3.56(dd) |
| (J_{5eq,5ax}) | (11.0) | (na,) | (na,) | (13.8) | (11.8) | (na) | (na) | (13.4) |
| H-6a | na | 4.08(dd) | 3.93(dd) | na | na | 4.21(dd) | 4.21(dd) | na |
| (J_{6a,6b,} J_{5,6a})) | | (12.7, 2.9) | (12.6, 2.4) | | | (12.8, 3.7) | (13.2, 3.8 | |
| H-6b | na | 4.03(dd) | 3.86(dd) | na | na | 4.12(dd) | 4.11(dd) | na |
| (J_{5,6b}) | | (2.8) | (2.4) | | | (2.8) | (2.6) | |
| H-1′a | 2.78(dd) | 3.57(br d) | 2.92(d) | 3.86(dd) | 3.93(dd) | 4.15(dd) | 3.98(dd) | 4.82(dd) |
| (J_{1′a,1′b,} J_{1′a,2′}) | (13.7, 2.8) | (14.1, 2.2) | (13.5, 2.6) | (14.4, 3.6) | (12.8, 3.7) | (13.9, 3.4) | (13.3, 6.3) | (12.8, 4.0) |
| H-1′b | 2.57(dd) | 3.15(br dd) | 2.88(dd) | 3.74(dd) | 3.83(dd) | 3.70(dd) | 3.85(dd) | 4.30(dd) |
| (J_{1′b,2′}) | (8.8) | (9.3) | (8.7) | (7.6) | (7.5) | 8.5) | (3.2) | (1.7) |
| H-2′ | 4.12(ddd) | 4.35(ddd) | 4.19(ddd) | 4.45(ddd) | 4.41(ddd) | 4.40(ddd) | 4.39(ddd) | 4.18(ddd) |
| (J_{2′,3′}) | (5.7) | (6.8) | (6.0) | (7.5) | (7.5) | (7.1) | (6.3) | (9.6) |
| H-3′ | 4.23(ddd) | 4.29(ddd) | 4.24(ddd) | 4.39(ddd) | 4.32(ddd) | 4.27(ddd) | 4.35(ddd) | 4.54(ddd) |
| (J_{3′,4′a}) | (3.5) | 3.4 | (3.4) | (3.6) | (3.4) | (3.6) | (2.7) | (5.3) |
| H-4′a | 3.87(dd) | 3.97(dd) | 3.91(dd) | 3.99(dd) | 4.94(dd) | 3.96(dd) | 3.97(dd) | 4.45(dd) |
| (J_{4′a,4′b}) | (12.6) | (12.7) | (12.2) | (12.9) | (12.6) | (12.5) | (12.6) | (10.6) |
| H-4′b | 3.80(dd) | 3.88(dd) | 3.85(dd) | 3.88(dd) | 3.84(dd) | 3.84(dd) | 3.84(dd) | 3.76(dd) |
| (J_{3′,4′b}) | (4.6) | (3.8) | (4.3) | (3.3) | (3.4) | (3.5) | (3.4) | (10.1) |

Footnotes for Table 3
^{a}500MHz, pH=10, D₂O.
^{b}500MHz, pH=8, Temp.=25° C., D₂O.
^{c}500MHz, pH=8, Temp.=40° C., D₂O.
^{d}600MHz, D₂O.
^{e}600MHz, D₂O.
^{f}500MHz, D₂O.
^{g}400MHz, D₂O.
^{h}600MHz, D₂O.
*Assignments for diastereotopic H-1/H-5 and H-2/H-4 pairs may be reversed.
na = not applicable,
n.d. = not determined

TABLE 4
¹³C NMR Data Compounds 66b, 67a, 67b, S-68b, R-68b, 69a,
69b and 70b.

| Com- | | | | | | | | |
|---|---|---|---|---|---|---|---|---|
| pound | 66b^{a}* | 67a^{b} | 67b^{b} | S-68b^{c}* | R-68b^{c}* | 69a^{c} | 69b^{c} | 70b^{c}* |
| C-1 | 58.19 | 55.85 | 56.48 | 41.25 | 43.24 | 43.62 | 41.76 | 39.29 |
| C-2 | 69.96 | 67.67 | 78.62 | 69.09 | 69.85 | 69.69 | 69.64 | 70.64 |
| C-3 | 78.77 | 77.60 | 78.14 | 74.05 | 78.10 | 78.57 | 78.58 | 78.42 |
| C-4 | 70.00 | 79.04 | 69.68 | 69.05 | 69.81 | 70.95 | 70.91 | 70.64 |
| C-5 | 57.64 | 66.90 | 66.29 | 41.01 | 43.13 | 61.19 | 60.88 | 39.10 |
| C-6 | na | 56.57 | 56.91 | na | na | 58.69 | 58.63 | na |
| C-1′ | 59.38 | 54.65 | 54.98 | 45.44 | 50.39 | 49.41 | 49.01 | 50.20 |
| C-2′ | 67.82 | 66.90 | 65.99 | 67.97 | 69.09 | 68.54 | 67.04 | 68.02 |
| C-3′ | 81.92 | 81.15 | 81.55 | 82.29 | 62.33 | 83.15 | 83.03 | 83.03 |
| C-4′ | 60.20 | 60.03 | 60.16 | 62.01 | 62.04 | 62.10 | 62.11 | 62.30 |

Footnotes for Table 4
^{a}125MHz, pH=10, D₂O.
^{b}125MHz, pH=8, D₂O.
^{c}100MHz D₂O.
*Assignments for diastereotopic C-1/C-5 and C-2/C-4 pairs may be reversed.
na = not applicable

### 5.2.8 Example 8

### Synthesis of Chain-Extended Homologues of Salacinol (Schemes 22 to 25)

General Procedure

A mixture of sulfide 117 and cyclic sulfate 105 in HFIP (1.0-3.0 mL/mmol of sulfide 117) was placed in a sealed reaction vessel and warmed with stirring for the indicated time at the temperature given below. The progress of the reaction was followed by TLC analysis of aliquots (developing solvent CHCl₃:MeOH, 10:1). When the limiting starting compound had been essentially consumed, the mixture was cooled, then diluted with CH₂Cl₂ and evaporated to a syrupy residue. Purification by column chromatography (CHCl₃ to CHCl₃:MeOH, 10:1) gave the purified sulfonium salt 119.

### Benzyl 2,3-Di-O-benzyl-4-O-sulfoxy-6-deoxy-6-[2,3,5-tri-O-benzyl-1,4-dideoxy-1,4-episufoniumylidene-D-arabinitol]-β-D-galactopyranose Inner Salt (119)

Reaction of sulfide 117 (431 mg, 1.02 mmol) with cyclic sulfate 105 (588 mg, 1.15 mmol) in HFIP (3.0 mL) for 42 h at 70° C. gave compound 119 as a colorless gummy solid (571 mg, 60%). [α]_{D}−7.6° (c 1.1, CHCl₃). See Tables 5 and 6 for NMR data. MALDI MS m/e 955.39 (M⁺+Na), 853.42 (M⁺+H−SO₃). Anal. Calcd for C₅₃H₅₆O₁₁S₂: C, 68.22; H, 6.05. Found: C, 68.48; H, 6.09.

### 1,4-Dideoxy-1,4-[[2R,3R,4R,5S-2,4,5,6-tetrahydroxy-3-(sulfooxy)hexyl]episufonium-ylidene]-D-arabinitol (95)

The protected sulfonium salt 119 (460 mg, 0.493 mmol) was dissolved in MeOH (50 mL) and stirred at rt with 10% Pd/C catalyst (580 mg) under 1 atm. of H₂ for 24 h. Analysis by TLC (EtOAc:MeOH:H₂O, 6:3:1) showed formation of a single product (rf 0.10). The catalyst was removed by filtration, using additional MeOH, through Celite and the filtrate was evaporated to give the crude hemiacetal compound 4-O-sulfoxy-6-deoxy-6-[1,4-dideoxy-1,4-episufoniumylidene-D-arabinitol]-α/β-D-galactopyranose inner salt (99, α:β=1:1) as a colorless foam (184 mg, 95%). See Tables 7 and 8 for NMR data of 99. MALDI MS m/e 414.89 (M⁺+Na), 392.93 (M⁺+H), 312.93 (M⁺+H−SO₃).

Reduction of hemiacetal 99 (430 mg, 1.10 mmol) with NaBH₄ as described above for compound 98 gave sulfonium sulfate 95 (232 mg, 54%) as a colorless glass. [α]_{D}+18° (c 0.72, MeOH). See Tables 9 and 10 for NMR data of 95. MALDI MS m/e 416.94 (M⁺+Na), 315.03 (M⁺+H−SO₃); HRMS. Calcd for C₁₁H₂₂O₁₁S₂Na (M−Na): 417.0501. Found: 417.0498.

TABLE 5
¹H NMR Data for Compound 119

| Compound | 119^{a} |
|---|---|
| H-1a | 4.07(br d) |
| (J_{1a,1b,} J_{1a,2}) | (13.3, <1) |
| H-1b | 3.59(dd) |
| (J_{1b,2}) | (3.7) |
| H-2 | 4.27(br d) |
| (J_{2,3}) | (˜2) |
| H-3 | 4.32(br s) |
| (J_{3,4}) | (<1) |
| H-4 | 3.91(dd) |
| (J_{4,5a}) | (6.3) |
| H-5a | 3.80(dd) |
| (J_{5a,5b}) | (9.6) |
| H-5b | 3.63(t) |
| (J_{4,5b}) | (9.8) |
| H-1′ | 4.45(d) |
| (J_{1′,2′}) | (7.6) |
| H-2′ | 3.80(dd) |
| (J_{2′,3′}) | (9.6) |
| H-3′ | 3.55(dd) |
| (J_{3′,4′}) | (3.4) |
| H-4′ | 5.00(dd) |
| (J_{4′,5′a}) | (1.2) |
| H-5′a | — |
| (J_{5′a,5′b}) | |
| H-5′b | — |
| (J_{4′,5′b}) | |
| H-5′ | 3.99(br m) |
| (J_{5′,6′a}) | (2.3) |
| H-6′a | 4.35(dd) |
| (J_{6′a,6′b}) | (13.0) |
| H-5′b | 3.94(dd) |
| (J_{5′,6′b}) | (5.6) |

Footnote for Table 5
^{a}Others: 7.45-7.02(30H, m, Ar), 5.03 and 4.56(2H, 2d, J_{A,B}=12.0Hz, CH₂Ar), 4.78 and 4.55(2H, 2d, J_{A,B}=12.2Hz, CH₂Ar), 4.76 and 4.71(2H, 2d, J_{A,B}=11.3Hz, CH₂Ar), 4.54 and 4.52(2H, 2d, J_{A,B}=12.2Hz, CH₂Ar), 4.33 and 4.20(2H, 2d, J_{A,B}=11.8Hz, CH₂Ar), 4.25 and 4.19(2H, 2d, J_{A,B}=12.0Hz, CH₂Ar).

TABLE 6
¹³C NMR Data for Compounds 119

| Compound | 119^{a} |
|---|---|
| C-1 | 47.65 |
| C-2 | 81.94 |
| C-3 | 83.16 |
| C-4 | 65.81 |
| C-5 | 66.72 |
| C-1′ | 103.20 |
| C-2′ | 77.52 |
| C-3′ | 78.28 |
| C-4′ | 72.98 |
| C-5′ | 69.21 |
| C-6′ | 50.09 |

Footnote for Table 6
^{a}Others: 138.61, 138.55, 137.25, 136.86, 136.22 and 135.83(6 × Cᵢₚₛₒ, Ar), 128.87-127.15(30C, Ar), 75.37, 73.51, 72.34, 71.68, 71.59 and 71.36(6 × CH₂Ar).

TABLE 7
¹H NMR Data for Compounds 99α and 99β

| Compound | 99α^{a} | 99β^{a} |
|---|---|---|
| H-1a | 3.87(m) | 3.87(m) |
| (J_{1a,1b}, J_{1a,2}) | (n.d., 3.9) | (n.d., 3.9) |
| H-1b | 3.87(m) | 3.87(m) |
| (J_{1b,2}) | (3.9) | (3.9) |
| H-2 | 4.73(ddd) | 4.73(ddd) |
| (J_{2,3}) | (3.9) | (3.7) |
| H-3 | 4.44(dd) | 4.44(dd) |
| (J_{3,4}) | (3.7) | (3.7) |
| H-4 | 4.06(dd) | 4.08(dd) |
| (J_{4,5a}) | (5.0) | (5.0) |
| H-5a | 4.11(dd) | 4.13(dd) |
| (J_{5a,5b}) | (11.8) | (12.2) |
| H-5b | 3.95(dd) | 3.96(dd) |
| (J_{4,5b}) | (n.d.) | (n.d.) |
| H-1′ | 5.32(d) | 4.65(d) |
| (J_{1′,2′}) | (3.9) | (7.9) |
| H-2′ | 3.83(dd) | 3.52(dd) |
| (J_{2′,3′}) | (10.4) | (10.1) |
| H-3′ | 4.02(dd) | 3.81(dd) |
| (J_{3′,4′}) | (3.3) | (3.3) |
| H-4′ | 4.74(d) | 4.68(dd) |
| (J_{4′,5′a}) | (<1) | (0.9) |
| H-5′a | — | — |
| (J_{5′a,5′b}) | | |
| H-5′b | — | — |
| (J_{4′,5′b}) | | |
| H-5′ | 4.67(m) | 4.30(ddd) |
| (J_{5′6′a}) | (n.d.) | (7.6.) |
| H-6′a | 3.95(dd) | 3.97(dd) |
| (J_{6′a,6′b}) | (n.d.) | (12.2.) |
| H-5′b | 3.92(dd) | 3.94(dd) |
| (J_{5′,6′b}) | (n.d.) | (4.0.) |

Footnotes for Table 7
n.d. not determined,
^{a}Temperature 313 K, D₂O,

TABLE 8
¹³C NMR Data for Compounds 99α and 99β

| Compound | 99α^{b} | 99β^{b} |
|---|---|---|
| C-1 | 49.27 | 49.27 |
| C-2 | 78.35ⁱ | 78.29ⁱ |
| C-3 | 79.26 | 79.06 |
| C-4 | 71.43 | 71.26 |
| C-5 | 60.62 | 60.62 |
| C-1′ | 94.07 | 98.17 |
| C-2′ | 69.35 | 72.85 |
| C-3′ | 69.17 | 72.85 |
| C-4′ | 79.63 | 78.65 |
| C-5′ | 66.88 | 60.91 |
| C-6′ | 48.73 | 48.30 |

Footnotes for Table 8
^{a}Temperature 308 K, D₂O,
^{b}Temperature 313 K, D2O,
^{e f g h i j k l m n} Assignments may be interchanged for resonances with the same superscript letter.

TABLE 9
¹H NMR Data for Compound 95

| Compound | 95^{a} |
|---|---|
| H-1a | 3.92(d) |
| (J_{1a,1b,} J_{1a,2}) | (n.d., 3.6) |
| H-1b | 3.92(d) |
| (J_{1b,2}) | (3.6) |
| H-2 | 4.78(dt) |
| (J_{2,3}) | (3.6) |
| H-3 | 4.47(dd) |
| (J_{3,4}) | (2.9) |
| H-4 | 4.11(ddd) |
| (J_{4,5a}) | (4.8) |
| H-5a | 4.16(dd) |
| (J_{5a,5b}) | (11.2) |
| H-5b | 3.91(dd) |
| (J_{4,5b}) | (8.1) |
| H-1a^{′} | 3.99(dd) |
| (J_{1′a,1′b,} J_{1′a′,2′}) | (13.3, 9.8) |
| H-1′b | 3.92(dd) |
| (J_{1′b,2′}) | (3.6) |
| H-2′ | 4.63(ddd) |
| (J_{2′,3′}) | (1.3) |
| H-3′ | 4.45(dd) |
| (J_{3′,4′a}) | (9.2) |
| H-4′ | 3.98(dd) |
| (J_{4′,5a′}) | 1.0 |
| H-4′b | — |
| (J_{4′a,4′b,} J_{3′,4′b}) | |
| H-5′a | — |
| (J_{5′a,5′b}) | |
| H-5′b | — |
| (J_{4′,5′b}) | |
| H-5′ | 4.03(t) |
| (J_{5′,6′a}) | (6.6) |
| H-6′a | 3.71(d) |
| (J_{6′a,6′b}) | (n.d.) |
| H-6′b | 3.71(d) |
| (J_{5′,6′b}) | (6.6) |

Footnotes for Table 9
n.d. not determined
^{a}Temperature 318 K, D2O

TABLE 10
¹³C NMR Data for Compound 95

| Compound | 95^{a} |
|---|---|
| C-1 | 48.49 |
| C-2 | 77.58 |
| C-3 | 78.32 |
| C-4 | 70.40 |
| C-5 | 59.85 |
| C-1′ | 51.15 |
| C-2′ | 66.58 |
| C-3′ | 78.89 |
| C-4′ | 769.36 |
| C-5′ | 70.12 |
| C-6′ | 63.40 |

Footnotes for Table 10
^{a}Temperature 318 K, D2O

### 5.3 Example 9

### Enzyme Inhibition Assays

5.3.1 In Vitro Inhibition Assays of Non-Human Glycosidase Enzymes

Various isomers of Salacinol, Blintol, Ghavamiol, and Acarbose were tested for their inhibition of three glycosidase enzymes, namely glucoamylase G2,^{19,20} porcine pancreatic α-amylase (PPA), and barley α-amylase (AMY1).²³ The results are summarized in Table 11. Glucoamylase G2 was weakly inhibited by Salacinol (1) (Ki=1.7 mM) whereas a stereoisomer of Blintol was a better inhibitor of this enzyme, with a Ki value of 0.72 mM. Salacinol (1) inhibited AMY1 and PPA, with Ki values of 15±1 and 10±2 μM, respectively. Other compounds did not significantly inhibit either AMY1 or PPA. It would appear then that Salacinol (1) and analogues of Salacinol (1) show discrimination for certain glycosidase enzymes, and are promising candidates for selective inhibition of a wider panel of enzymes that includes human small intestinal maltase-glucoamylase¹⁷ and human pancreatic α-amylase.¹⁸

The glucoamylase G2 form from Aspergillus niger was purified from a commercial enzyme (Novo Nordisk, Bagsvaerd, Denmark) as described.^{19, 20} The initial rates of glucoamylase G2-catalyzed hydrolysis of maltose was tested with 1 mM maltose as substrate in 0.1 M sodium acetate pH 4.5 at 45° C. using an enzyme concentration of 7.0×10⁻⁸ M and five inhibitor concentrations in the range 1 μm-5 mM. The effect of the inhibition on rates of substrate hydrolysis were compared for the different compounds. The glucose released was analyzed in aliquots removed at appropriate time intervals using a glucose oxidase assay adapted to microtiter plate reading and using a total reaction volume for the enzyme reaction mixtures of 150 or 300 μL.²¹ The Kᵢ values were calculated assuming competitive inhibition from 1/v=(1/Vmax)+[(Kₘ)/(Vmax[S]Kᵢ)][I], where v is the rate measured in the presence or absence of inhibitor, [1] and [S] the concentrations of inhibitor and substrate, Kₘ 1.6 mM and kcat 11.3 s⁻¹, using ENZFITTER.²²

Porcine pancreatic α-amylase (PPA) and bovine serum albumin (BSA) were purchased from Sigma. Amylose EX-1 (DP17; average degree of polymerization 17) was purchased from Hayashibara Chemical Laboratories (Okayama, Japan). Recombinant barley α-amylase isozyme 1 (AMY1) was produced and purified as described.²³ An aliquot of the porcine pancreatic α-amylase (PPA) crystalline suspension (in ammonium sulfate) was dialyzed extensively against the assay buffer without BSA. The enzyme concentration was determined by aid of amino acid analysis as determined using an LKB model Alpha Plus amino acid analyzer. The inhibition of AMY1 (3×10⁻⁹ M) and PPA (9×10⁻⁹ M) activity towards DP17 amylose was measured at 37° C. in 20 mM sodium acetate, pH 5.5, 5 mM CaCl₂, 0.005% BSA (for AMY1) and 20 mM sodium phosphate, pH 6.9, 10 mM NaCl, 0.1 mM CaCl₂, 0.005% BSA (for PPA). Six different final inhibitor concentrations were used in the range 1 μM-5 mM. The inhibitor was pre-incubated with enzyme for 5 min at 37° C. before addition of substrate. Initial rates were determined by measuring reducing sugar by the copper-bicinchoninate method as described.^{23,24} The Kᵢ values were calculated assuming competitive inhibition, as described above for the case of glucoamylase, and a Kₘ of 0.57 mg/ml and kcat of 165 s⁻¹ for AMY1 and 1 mg/ml and 1200 s⁻¹ for PPA, as determined in the substrate concentration range 0.03-10 mg/ml using ENZFITTER.²² For the Kᵢ determinations, [S]=0.7 mg/mL amylose DP 17 for the AMY1 binding and [S]=2.5 mg/mL amylose DP 17 for the PPA binding.

5.3.2 In Vitro Inhibition Assays of Human Glycosidase Enzymes

The in vitro inhibitory activities of Salacinol, Blintol, Ghavamiol, and Acarbose were tested against human glycosidase enzymes as described below.

5.3.2.1 Enzyme Assays with Maltase Glucoamylase (MGA)

Since recombinant MGA enzyme has not been expressed successfully, the assay for MGA activity measured effects on cell extracts. In the assays, COS cells transfected with MGA5′ (maltase subunit clone 10) construct were used. Activity measurements were performed with cell extracts containing MGA. Maltose hydrolysis was monitored by measurement of the glucose released by a glucose oxidase colorimetric assay. Inhibition of this hydrolysis was measured as a reduction in OD reading. Since the assay deals with cell extracts, a standard inhibitor, e.g. Salacinol, is always included in each new assay of a putative inhibitor.

In practice, an OD reading in the absence of the inhibitor was recorded, followed by a reading in the presence of the inhibitor. The percent reduction in OD reading upon administering a candidate inhibitor (see Table 11) was then correlated with a percent inhibition for a given concentration. For example: 1) at 200 nM (0.2 μM), Blintol inhibits 50% of MGA activity whereas Salacinol inhibits-50% of MGA activity at 2500 nM (2.5 μM); 2) Whereas Salacinol at 5 μM concentration inhibits 60% of the breakdown of maltose, Acarbose only inhibits 4% of the activity.

5.3.2.2 Enzyme Assays with Human Pancreatic α-Amylase (HPA)

These assays were performed with purified enzyme. The ability of candidate inhibitors to inhibit the hydrolysis of 2,4-Dinitrophenyl maltotrioside was monitored by UV-visible spectroscopy of the released 2,4-dinitrophenol.

5.3.2.3 Summary of In Vitro Biological Activity

1) It appears that Acarbose acts principally by inhibiting human pancreatic α-amylase (HPA) and the breakdown of starch. Salacinol inhibits both HPA and MGA and Blintol appears to only MGA.

2) The selenium analogue of Salacinol, Blintol, shows inhibition of MGA at lower concentrations than Salacinol. More significantly, Blintol does not appear to inhibit HPA. Salacinol, on the other hand, inhibits both HPA and MGA, and Acarbose inhibits only HPA in these experimental assays.

3) Using similar monitoring of OD readings as in the MGA assay, the maltase activity in biopsies with live intestinal cells was monitored. At 5 μM concentration, Blintol inhibits 50% of maltase activity whereas Salacinol inhibits only 13% of the activity.

TABLE 11
Summary of Activity of Salacinol Derivatives Against Human
Glycosidases

| | Ki (mM) | | | | |
|---|---|---|---|---|---|
| Compound | AMY1^{a} | PPA^{b} | HPA^{c} | GA^{d} | MGA^{e} |
| 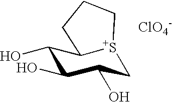 | >5 | >5 | — | 1.32 | — |
| 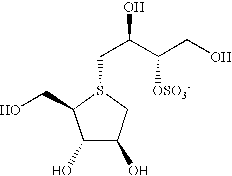 AG-1 | 0.015 | 0.01 | 0.075 | 1.71 | μM |
| 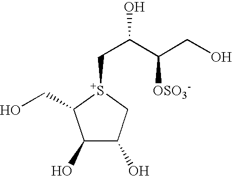 AG-2 | >5 | >5 | n.a. | 2.17 | n.a. |
| 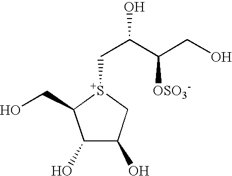 AG-3 | >5 | >5 | n.a. | 1.06 | mM |
| 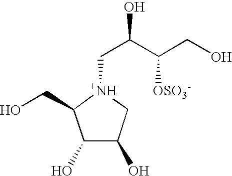 AG-4 | >5 | >5 | n.a. | >2.5 | mM |
| 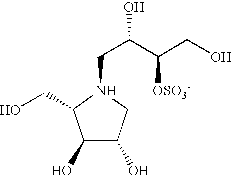 AG-5 | >5 | >5 | 0.4 | >8 | n.a. |
| 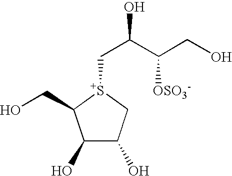 AG-6 | 0.109 | 0.052 | — | >5 | — |
| 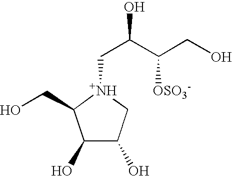 AG-7 | >5 | >5 | — | >30 | — |
| 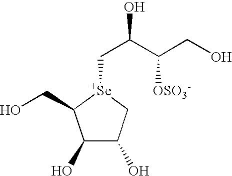 BJ-24-77-3, BJ-24- 78-1, BJ-24-79-1 | >5 | >5 | >5 | 0.72 | nM |
| 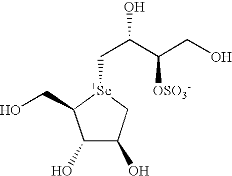 BJ-24-92-1 | >5 | >5 | n.a. | >9 | n.a. |
| 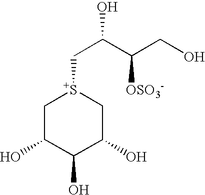 MS-02-159 | — | — | >5 | — | n.a. |
| 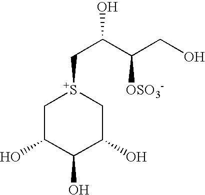 MS-02-153 | — | — | >5 | — | mM |
| 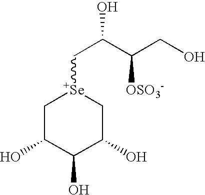 MS-02-145 | — | — | >5 | — | n.a. |
| 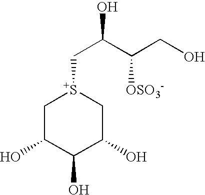 MS-03-119 | — | — | — | — | n.a. |
| 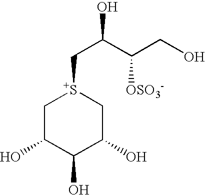 MS-03-125 | — | — | — | — | n.a. |
| 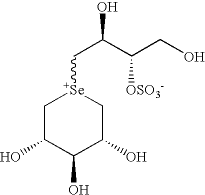 MS-03-163B | — | — | — | — | n.a. |
| 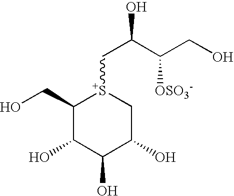 MS-03-175 | — | — | — | — | n.a. |
| 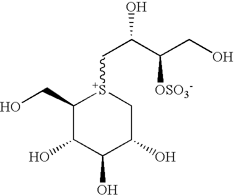 MS-03-171 | — | — | — | — | n.a. |

^{a}AMY1 = Barley α-amylase
^{b}PPA = Porcine pancreatic α-amylase
^{c}HPA = Human pancreatic α-amylase
^{d}GA = Glucoamylase
^{e}MGA = Human intestinal maltase glucoamylase
n.a. = not active
— = not tested

5.3.3 In Vivo Inhibition Studies of Blintol and Salacinol

In this Example the efficacy of Blintol in inhibiting glucose absorption and lowering post-prandial glucose levels in vivo was compared to Salacinol and Acarbose.

Five week old Sprague-Dawley rats were housed singly under a 12:12-h light-dark photoperiod and given free access to water and rat chow (Purina rodent chow). After one week of acclimation, chronic indwelling catheters were implanted. Animals were anesthetized with a combination of Ketamine-Xylazine-Butorphanol (0.1 ml/100 g im). Analgesic (Butorphenol, 1 mg/kg sc) was administered following recovery from anesthesia and the following morning. Antibiotic was administered by one dose sc and in the drinking water for 4 days post-operative (Baytril, 5 mg/kg sc, Bayer, Toronto, Canada; Baytril, 50 mg/ml: 0.36 ml solution in 250 ml drinking water). A sterile catheter (Intramedic PE-50 with ˜3 cm beveled Silastic tip) was placed in the left carotid artery and the distal end of the catheter was tunneled subcutaneously, exteriorized, and anchored at the nape of the neck. The catheters were protected from chewing by a stainless steel tether connected to a swivel system which allowed free movement of the animal and easy access to the catheter by the investigator.

The animals were allowed to recover for 1 week. Experiments were performed on conscious, unrestrained animals that had been fasted overnight by removal of chow from the cage hoppers at 2100. At 0800 the following morning animals were weighed and Atropine (0.05 mg/kg sc) was administered as a muscle relaxant. At baseline, animals were administered a bolus of maltose by oral gavage (1000 mg/kg body weight) with or without drug (25 mg/kg body weight for all agents). Blood samples (0.1 mL) were taken via the implanted carotid line at −15 and −5 min for the baseline and at 7, 15, 30, 60, 90, 120, 210, 300 min.

Blood samples were kept on ice in microcentrifuge tubes and then were centrifuged. The plasma was stored at −20° C. until it was assayed. Plasma volume was triple replaced with heparinized saline (10u/mL), but red blood cells were not reinfused. Plasma glucose was assayed with the glucose oxidase method (Trinder RAICHEM Division of Hemagen Diagnostics, Inc. San Diego, Calif.). Plasma insulin concentrations were measured by rat insulin ELISA (Crystal Chem INC, Downers Grove, Ill.). Six experiments were performed for each treatment (control, Blintol, Acarbose, Salacinol).

The AUC (Area Under the Curve) was calculated for glucose, glucose absorption, and insulin by applying the trapezoidal method over the 0 to 90 minute time points. For glucose the AUC was calculated for the excursion from each sample above the basal value (average of the −5 and −15 minute samples), and for insulin and glucose absorption for the excursion above 0.

All data are presented as means±SE. The significance of changes in plasma glucose and insulin were tested by two-way repeated-measures analysis of variance and were performed with the Statistical Analysis System for Windows (version 6.3, SAS Institute, Cary, N.C.). AUCs were compared using unpaired t-tests.

5.3.3.1 Plasma Glucose Concentrations

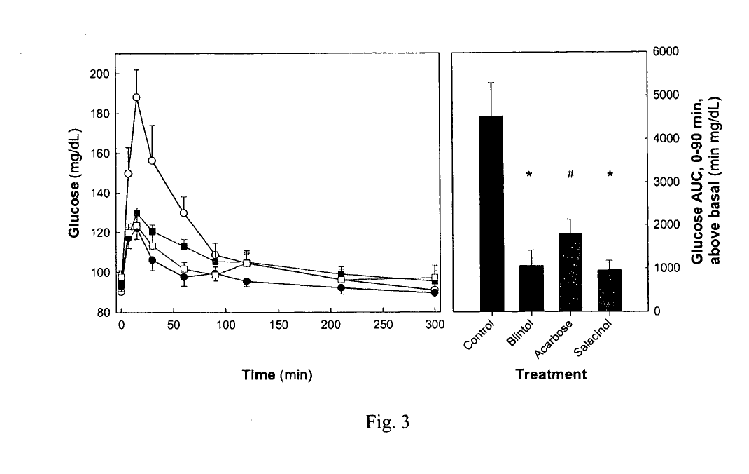

The plasma glucose profiles for all treatments were significantly lower than Control (P<0.0001; all treatments versus Control), and the Blintol group had a lower profile than Acarbose (P<0.01) but there was no difference between other treatments (see FIG. 3). Plasma glucose concentrations for all groups increased immediately following gavage (P<0.01), reaching a peak at 15 minutes. For the Control group, the 15 minute glucose excursion from basal was 98.0±12.4 mg/dL and this excursion was decreased with all treatments (Blintol: 29.3±6.5, Acarbose: 34.2±3.5, Salacinol: 26.0±5.1; P<0.005). All groups exhibited an exponential glucose decay following the 15 minute peak. For the control group glucose values did not return to basal values until 210 minutes (P=0.46). Blintol (P=0.40) and Salacinol (P=0.43) groups returned to basal at 60 minutes, and Acarbose at 90 minutes (P=0.19).

The Area Under the Curve was significantly decreased with all treatments. Blintol and Salacinol yielded slightly lower 90 minute AUC's than Acarbose, though the difference was not significant (Blintol: P=0.16, Salacinol: P=0.6; versus Acarbose).

5.3.3.2 Plasma Insulin Concentrations

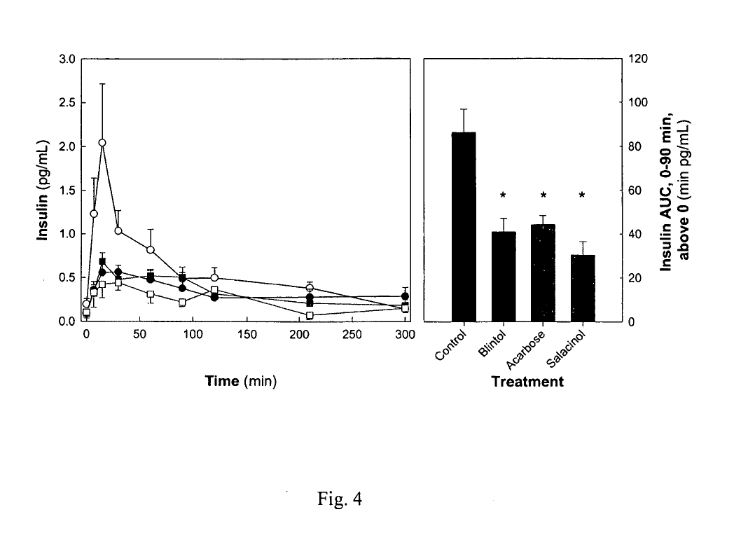

Plasma insulin profiles were decreased with all treatments versus Control (P<0.0001) (FIG. 4). Consistent with the peak glucose at 15 minutes, the insulin for all groups was also peaked between 7 and 15 minutes; however the insulin profile was more rounded and did not show an exponential decay for any group. While the Control and Acarbose insulin values were different from basal for the 15 to 90 minute range, the Blintol and Salacinol insulin values were only different from basal at 15 minutes (P<0.05). The 90 minute insulin AUC was decreased with Blintol, Acarbose, and Salacinol treatments by 53%, 49%, and 65% respectively. There was no statistical difference between treatment groups.

The results of this experiment show that Blintol, Acarbose, and Salacinol, at a 25 mg/kg body weight dosage significantly lower post-prandial plasma glucose and insulin concentrations in normal, catheterized rats. Importantly, at this dosage, Blintol had a decreased glucose profile compared to Acarbose. The improvement in the glucose profile with all treatments seems to be directly attributable to an inhibited glucose absorption, consistent with the agents' expected mechanisms of action.

The inhibition of the post-prandial glucose peak observed with all treatments may contribute to a reduction in diabetic complications when these agents are used chronically. The reduced glucose levels decreased the demand on the insulin secreting β-cells and chronically may contribute to a preservation of β-cell mass and function. Moreover, the better controlled glucose levels may decrease a glucose-toxic effect which can kill or impair the function of the insulin-secreting β-cells. Chronic administration of drug studies will help elucidate if these factors are able to slow or prevent the onset of diabetes in a diabetes-prone animal model.

As will be apparent to those skilled in the art in the light of the foregoing disclosure, many alterations and modifications are possible in the practice of this invention without departing from the spirit or scope thereof.

### REFERENCES

1. Yoshikawa, M. et al. Tetrahedron Lett. 1997, 38(48), 8367-8370.
2. Yoshikawa, M. et al. Chem. Pharm. Bull. 1998, 46(8), 1339-1340.
3. Shimoda, H. et al. Journal of the Food Hygienic Society of Japan. 1999, 40(3), 198-205.
4. Goss, P. E. et al. Clinical Cancer Res. 1997, 3, 1077-1086.
5. Mohla, S. et al. Anticancer Res. 1990, 10, 1515-1522.
6. Goss, P. E. et al. Cancer Res. 1994, 54, 1450-1457.
7. Eames, J. et al. Tetrahedron Lett. 1998, 39(10), 1247-1250.
8. Calvo-Flores, F. G. et al. J. Org. Chem. 1997, 62, 3944-3961.
9. Foster, A. B. et al. J. Chem. Soc. 1961, 5005-5011.
10. MacDonald, D. L. et al. J. Am. Chem. Soc. 1956, 78, 3720-3722.
11. Yoshimura, Y. et al. J. Org. Chem. 1997, 62, 3140-3152.
12. Satoh, H. et al. Bioorg. Med. Chem. Lett. 1998, 8(9), 989-992.
13. Fleet, G. et al. Tetrahedron. 1986, 42, 5685-5692.
14. Reichardt, C. Solvents and Solvent Effects in Organic Chemistry, 2nd Ed.; VCH: Weinheim, 1996; pp 137-147, 359-384.
15. Yuasa, H.; Takada, J., Hashimoto, H. Tetrahedron Lett. 2000, 41, 6615-6618.
16. Ghavami, A.; Johnston, B. D.; Jensen, M. T.; Svensson, B.; Pinto, B. M. J. Am. Chem. Soc. 2001, 123, 6268-6271.
17. Nichols, B. L.; Eldering, J.; Avery, S.; Hahn, D.; Quaroni, A.; Sterchi, E. J. Biol. Chem. 1998, 273, 3076-3081.
18. Braun, C.; Brayer, G. D.; Withers, S. G. J. Biol. Chem. 1995, 270, 26778-26781.
19. Svensson, B.; Pedersen, T.; Svendsen, I.; Sakai, T.; Ottesen, M. Carlsberg Res. Commun. 1982, 47, 55-69.
20. Stoffer, B.; Frandsen, T. P.; Busk, P. K.; Schneider, P.; Svendsen, I.; Svensson, B. Biochem J. 1993, 292,197-202.
21. Frandsen, T. P.; Dupont, C.; Lehmbeck, J.; Stoffer, B.; Sierks, M. R.; Honzatko, R. B.; Svensson, B. Biochemistry 1994, 33, 13808-13816.
22. Leatherbarrow, R. J. Enzfitter, a nonlinear regression data analysis program for IBM PC; Elsevier Science Publishers BV: Amsterdam, The Netherlands, 1987.
23. Juge, N.; Andersen, J. S.; Tull, D.; Roepstorff, P.; Svensson, B. Protein Expression Purif. 1996, 8, 204-214.
24. Fox, J. D.; Robyt, J. F. Anal. Biochem. 1991, 195, 93-96.
25. Ghavami, A. Johnston, B. D.; Pinto, B. M. J. Org. Chem. 2001, 66,2312-2317.
26. Johnston, B. D.; Ghavami, A.; Jensen, M. T.; Svensson, B.; Pinto, B. M. J. Am. Chem. Soc. 2002, 124, 8245-8250.
27. Yoshikawa, M.; Morikawa, T.; Matsuda, H.; Tanabe, G.; Muraoka, O. Bioorg. Med. Chem., 2002, 10,1547-1554.
28. Svansson, L.; Johnston, B. D.; Gu, J.-H.; Patrick, B.; Pinto, B. M. J. Am. Chem. Soc., 2000, 122, 10769-10775.
29. Ghavami, A.; Johnston, B. D.; Maddess, M. D.; Chinapoo, S. M.; Jensen, M. T.; Svensson, B.; Pinto, B. M. Can. J. Chem., 2002, 80, 937-942.
30. Wakabayashi, T.; Mori, K.; Kobayashi, S. J. Am. Chem. Soc. 2001, 123, 1372-1375.
31. Crivello, J. V. Advances in Polymer Science 1984, 62, 1-48.
32. Trost, B. M.; Melvin, L. S., Jr. Organic Chemistry, Vol. 31: Sulfur Ylides, Emerging Synthetic Intermediates, 1975.
33. Fox, D. J.; House, D.; Warren, S. Angew. Chem., nt. Ed. 2002, 41, 2462-2482.
34. Stutz, A. E. Iminosugars as Glycosidase Inhibitors: Nojirimycin and Beyond, 1999.
35. Izquierdo, I.; Plaza, M. T.; Aragon, F. Tetrahedron: Asymmetry 1996, 7, 2567-2575.
36. Ulgar, V.; Fernandez-Bolanos, J. G.; Bols, M. J. Chem. Soc., Perkin Trans. 1 2002, 1242-1246.
37. Bazin, H. G.; Linhardt, R. J. Synthesis 1999, 621-624.
38. Calvo-Asin, J. A.; Calvo-Flores, F. G.; Exposito-Lopez, J. M.; Hernandez-Mateo, F.; Garcia-Lopez, J. J.; Isac-Garcia, J.; Santoyo-Gonzalez, F.; Vargas-Berenguel, A. J. Chem. Soc., Perkin Trans. 1 1997, 1079-1081.
39. Bozo, E.; Boros, S.; Kuszmann, J.; Gacs-Baitz, E.; Parkanyi, L. Carbohydr. Res. 1998, 308, 297-310.
40. Dagron, F.; Lubineau, A. J. Carbohydr. Chem. 2000, 19, 311-321.
41. Bazin, H. G.; Wolff, M. W.; Linhardt, R. J. J. Org. Chem. 1999, 64, 144-152.
42. Yin, H.; D'Souza, F. W.; Lowary, T. L. J. Org. Chem. 2002, 67, 892-903.
43. Hatanaka, K.; Kuzuhara, H. J. Carbohydr. Chem. 1985, 4, 333-345.
44. Ness, R. K.; Fletcher, H. G., Jr. J. Am. Chem. Soc. 1958, 80, 2007-2010.
45. Fukuyama, Y.; Ciancia, M.; Nonami, H.; Cerezo, A. S.; Erra-Balsells, R.; Matulewicz, M. C. Carbohydr. Res. 2002, 337, 1553-1562.
46. Harris, S. L, L. Craig, J. S. Mehroke, M. Rashed, M. B. Zwick, K. Kenar, E. J. Toone, N. Greenspan, F.-I. Auzanneau, J.-R. Marino-Albernas, B. M. Pinto, and J. K. Scott. Proc. Natl. Acad. Sci. USA, 94, 2434 (1997).
47. B. M. Pinto, J. K. Scott, S. L. Harris, M. A. Johnson, D. R. Bundle, M. C. Chervenak, M. N. Vyas, N. K. Vyas, F. A. Quiocho. XIX International Carbohydrate Symposium, San Diego, U.S.A., (August, 1998), Abstr. CP 132, CO 019; B. M. Pinto. Meeting on anti-idiotypes and mimotopes in vaccine development, Vibo Valentia, Italy, (May, 2000); B. M. Pinto. Glycobiology Symposium, Pacifichem 2000 Conference, Honolulu, Hi., U.S.A., (December, 2000) Abstr. 607.
48. (a) B. Belleau, U. Gulini, B. J. Gour-Salin, and F. R. Ahmed. Can. J. Chem. 63, 1268 (1985); (b) B. Belleau, U. Gulini, R. Camicioli, B. J. Gour-Salin, and G. Sauvé. Can. J. Chem. 64, 110 (1986); (c) S. Lemaire, B. Belleau, and F. Jolicocur. Adv. Biosci. 75, 105 (1989); (d) F. B. Jolicoeur, D. Ménard, B. Belleau, and S. Lemaire. Int. Narcotics Res. Conf. 89, 49 (1990).
49. H. Yuasa, J. Takada, and H. Hashimoto. Bioorg. Med. Chem. Lett. 11, 1137 (2001).
50. O. Muraoka, S. Ying, K. Yoshikai, Y. Matsuura, E. Yamada, T. Minematsu, G. Tanabe, H. Matsuda, and M. Yoshikawa. Chem. Pharm. Bull. 49, 1503 (2001).
51. P. A. M. van der Klein, A. E. J. de Nooy, G. A. van der Marel, and J. H. van Boom. Synthesis 347, (1991).
52. P. A. M. van der Klein, W. Filemon, H. J. G. Broxterman, G. A. van der Marel, and J. H. van Boom. Synth. Commun. 22, 1763, (1992).
53. Ghavami, A.; Sadalapure, K. S.; Johnston, B. D.; Lobera, M.; Snider, B. B.; Pinto, B. M. Synlett 2003, 1259-1262.
54. Mootoo, D. R.; Date, V.; Fraser-Reid, B. J. Chem. Soc., Chem. Commun. 1987, 1462-1463.
55. Mootoo, D. R.; Date, V.; Fraser-Reid, B. J. Am. Chem. Soc. 1988, 110, 2662-2663.
56. Furneaux, R. H.; Rendle, P. M.; Sims, I. M. J. Chem. Soc., Perkin, Trans. 1, 2000, 2011-2014.
57. Guijar, M. K.; Reddy, L. K.; Hotha, S. J. Org. Chem. 2001, 66, 4657-4660.
58. Varki, A.; Cummings, R.; Esko, J.; Freeze, H.; Hart, G.; Marth, J., Eds. Essentials of Glycobiology; Cold Spring Harbor Laboratory Press: Cold Spring Harbor, N.Y., 1999.
59. Taylor, M. E.; Drickamer, K. Introduction to Glycobiology; Oxford University Press: New York, N.Y., 2003.
60. Helenius, A.; Aebi, M. Science. 2001, 2364-2369.
61. (a) Dwek, R. A.; Butters, T. D. Platt, F. M.; Zitzmann, N. Nature Rev. Drug Discovery, 2002, 1, 65-75. (b) Dwek, R. A. Chem. Rev. 1996, 96, 683-720.
62. For leading references: (a) Elbein, A. D.; Molyneux, R. J. Ch. 7 in Comprehensive Natural Products Chemistry, Vol 3; Pinto, B. M., Ed.; Barton, D. H. R.; Nakanishi, K.; Meth-Cohn, O., Ser. Eds.; Elsevier: UK, 1999. (b) Asano, N,; Nash, R. J.; Molyneux, R. J.; Fleet, G. W. J. Tetrahedron: Asymmetry 2000, 11, 1645-1680. (c) McCarter, J. D.; Withers, S. G. Curr. Opin. Struct. Biol. 1994, 4, 885-892. (d) Ly, H. D.; Withers, S. G. Annu. Rev. Biochem. 1999, 68, 487-522.
63. Bock, K.; Sigurskjold, B. Studies Nat. Prod. Chem. 1990, 7, 29-86; Holman, R. R.; Cull, C. A.; Turner, R. C. Diabetes Care, 1999, 22, 960-964; Jacob, G. S. Curr. Opin. Struct. Biol. 1995, 5, 605-611. Sigurskjold, B. W.; Berland; C. R.; Svensson, B. Biochemistry 1994, 33, 10191-10199.
64. Kapit, W.; Macey, R. I.; Meisami, E. The Physiology Coloring Book; Harper Collins College Publishers: Menlo Park, Calif., 1987.
65. Cox, T.; Lachmann, R.; Hollak, C.; Aerts, J.; van Weely, S.; Hrebicek, M.; Platt, F.; Butters, T.; Dwek, R.; Moyses, C.; Gow, I.; Elstein, D.; Zimran, A. Lancet 2000, 355, 1481-1485.
66. (a) Koshland, D. E. Biol. Rev. 1953, 28, 416-436. (b) Zechel, D. L.; Withers, S. G. Curr. Opin. Chem. Biol. 2001, 5, 643-649.
67. Lillelund, V. H.; Jensen, H. H.; Liang, X; Bols, M. Chemical Reviews 2002, 102, 515-553.
68. Varrot, A.; Tarling, C. A.; MacDonald, J. M.; Stick, R. V.; Zechel, D. L.; Withers, S. G.; Davies, G. J. J. Am. Chem. Soc. 2003, 125, 7496-7497.
69. Varrot, A.; MacDonald, J.; Stick, R. V.; Pell, G.; Gilbert, H. J.; Davies, G. J. Chem. Commun. 2003, 946-947.
70. Zechel, D. L.; Boraston, A. B.; Gloster, T.; Boraston, C. M.; MacDonald, J. M.; Tilbrook, D. M. G.; Stick, R. V.; Davies, G. J. J. Am. Chem. Soc. 2003, 125, 14313-14323.
71. Johnson, M. A.; Jensen, M. T.; Svensson, B.; Pinto, B. M. J. Am. Chem. Soc. 2003, 125, 5663-5670.
72. Ghavami, A.; Chen, J. J-W.; Pinto, B. M. Carbohydr. Res. 2004, 339, 401-407.
73. van den Broek, L. A. G. M.; Vermaas, D. J.; Heskamp, B. M.; van Boeckel, C. A. A.; Tan, M. C. A. A.; Bolscher, J. G. M.; Ploegh, H. L.; van Kemenade, F. J.; de Goede, R. E. Y.; Miedema, F. Recl. Trav. Chim. Pays-Bas 1993, 112, 82-94.
74. Asano, N.; Kizu, H.; Oseki, K.; Tomioka, E.; Matsui, K.; Okamoto, M.; Baba, M. J. Med. Chem. 1995, 38, 2349-56.
75. Hausler, H.; Rupitz, K.; Stutz, A. E.; Withers, S. G. Monatshefte fur Chem. 2002, 133, 555-560.
76. Halila, S.; Benazza, M.; Demailly, G. Tetrahedron Lett. 2001, 42, 3307-3310.
77. Klayman, D. L.; Griffin, T. S. J. Am. Chem. Soc. 1973, 95, 197-199.
78. Lucas, M. A. Nguyen, O. T. K.; Schiesser, C. H; Zheng, S.-L. Tetrahedron 2000, 56, 3995-4000.
79. Hashimoto, H.; Masashi, K.; Yuasa, H. Carbohydr. Res. 1996, 282, 207-222.
80. Svensson, B.; Sierks, M. R. Carbohydr. Res. 1992, 227, 29-44.
81. Gao, Y.; Sharpless, K. B. J. Am. Chem. Soc. 1988, 110, 7538-7539.

1. A non-naturally occurring compound selected from the group consisting of compounds represented by the general formula (I) and stereoisomers and pharmaceutically acceptable salts thereof: 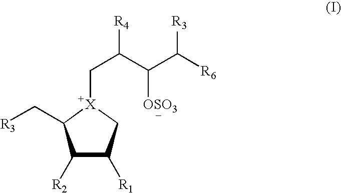
where X is selected from the group consisting of S, Se and NH; R₁, R₂, R₃, R₄ and R₅ are the same or different and are selected from the group consisting of H, OH, SH, NH₂, halogens and constituents of compounds selected from the group consisting of cyclopropanes, epoxides, aziridines and episulfides; and R₆ is selected from the group consisting of H and optionally substituted straight chain, branched, or cyclic, saturated or unsaturated hydrocarbon radicals.

2. The compound as defined in claim 1, wherein R₆ is an alditol side-chain.

3. The compound as defined in claim 1, wherein R₆ is a polyhydroxylated, acylic chain comprising between 5 and 10 carbons.

4. The compound as defined in claim 3, wherein said chain comprises 5 or 6 carbons.

5. The compound as defined in claim 3, wherein X═S and wherein said compound is a chain-extended homologue of Salacinol.

6. The compound as defined in claim 3, wherein R₁, R₂, R₃, R₄ and R₅ are OH.

7. The compound as defined in claim 3, wherein said compound is 1,4-Dideoxy-1,4-[[2R,3R,4R,5S-2,4,5,6-tetrahydroxy-3-(sulfooxy)hexyl]episufonium-ylidene]-D-arabinitol.

8. A compound selected from the group consisting of compounds represented by the general formula (II) and stereoisomers and pharmaceutically acceptable salts thereof: 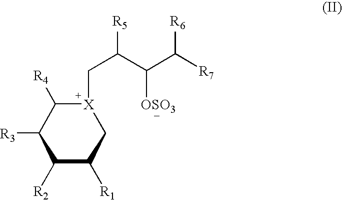
where X is selected from the group consisting of S, Se and NH; R₁, R₂, R₃, R₅ and R₆ are the same or different and are selected from the group consisting of H, OH, SH, NH₂, halogens and constituents of compounds selected from the group consisting of cyclopropanes, epoxides, aziridines and episulfides; R4 is selected from the group consisting of H and CH₂OH; and R₇ is selected from the group consisting of H and optionally substituted straight chain, branched, or cyclic, saturated or unsaturated hydrocarbon radicals.

9. The compound as defined in claim 8, wherein R₁, R₂, R₃, R₅ and R₆ are OH and R₄ and R₇ are H.

10. The compound as defined in claim 8, wherein R₁, R₂, R₃, R₅ and R₆ are OH, R₄ is CH₂OH and R₇ is H.

11. A process for synthesis of the compound (I) of claim 1 comprising:
(a) providing a cyclic sulfate having the general formula (III) 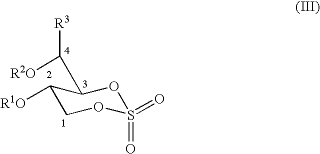
wherein R¹ and R² are H or comprise a protecting group and R³ is selected from the group consisting of H and optionally substituted straight chain, branched, or cyclic, saturated or unsaturated hydrocarbon radicals and their protected derivatives;
(b) providing a 5-membered heterocycle ring compound of the general formula (IV), 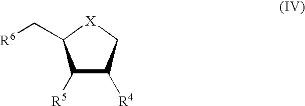
wherein X is selected from the group consisting of S, Se, and NH and R⁴, R⁵ and R⁶ are selected from the group consisting of OH and a protected hydroxyl group;
(c) reacting the cyclic sulfate with the 5-membered heterocycle ring compound to produce an intermediate compound having an internal salt structure comprising a positively charged heteroatom X and a negatively charged sulfate counterion; and
(d) removing any protecting groups from said intermediate compound.

12. The process as defined in claim 11, wherein said reacting of said cyclic sulfate and said heterocycle ring compound is performed in a polar solvent.

13. The process as defined in claim 12, wherein said polar solvent comprises hexafluoroisopropanol.

14. The process as defined in claim 1, wherein said cyclic sulfate is derived from D-glucose.

15. The process as defined in claim 11, wherein said cyclic sulfate is a benzylidene-protected cyclic sulfate.

16. The process as defined in claim 15, wherein one or more benzylidene protecting groups are installed on said benzylidene-protected cyclic sulfate in the presence of the catalyst pyridinium—p-toluenesulfonate (PPTS).

17. The process as defined in claim 11, wherein said protected hydroxyl group of said heterocycle ring compound is p-methoxybenzyl.

18. The process as defined in claim 11, wherein said heterocycle ring compound is p-methoxybenzyl-protected 1,4-anhydro-4-seleno-D-arabinitol.

19. The process as defined in claim 11, wherein the removal of the protecting groups is performed by hydrogenolysis of said intermediate compound.

20. The process as defined in claim 11, wherein the removal of the protecting groups is performed by acid hydrolysis.

21. The process as defined in claim 20, wherein said acid hydrolyis is performed with trifluoroacetic acid.

22. The process as defined in claim 11, wherein said heterocycle ring compound is derived from L-xylose.

23. The process as defined in claim 11, wherein said intermediate compound comprises a sulfonium-sulfate disaccharide analogue.

24. The process as defined in claim 11, wherein said intermediate compound is a sulphonium sulfate derivative of a monosaccharide selected from the group consisting of glucose, galactose, arabinose and xylose.

25. The process as defined in claim 1 further comprising reducing said intermediate compound with sodium borohydride to yield the target compound (I).

26. A process for synthesis of a compound (II) according to claim 8 comprising:
(a) providing a cyclic sulfate having the general formula (III) 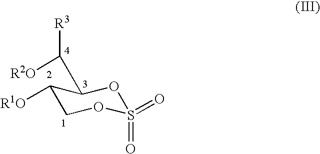
wherein R¹ and R² are H or comprise a protecting group and R³ is selected from the group consisting of H and optionally substituted straight chain, branched, or cyclic, saturated or unsaturated hydrocarbon radicals and their protected derivatives;
(b) providing a 6-membered heterocycle ring compound of the general formula (V), 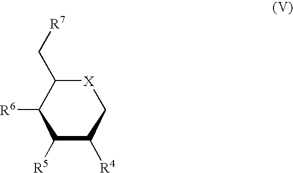
wherein X is selected from the group consisting of S, Se, and NH and R⁴, R⁵, R⁶ and R⁷ are selected from the group consisting of OH or a protected hydroxyl group;
(c) reacting the cyclic sulfate with the heterocycle ring compound to produce an intermediate compound having an internal salt structure comprising a positively charged heteroatom X and a negatively charged sulfate counterion; and
(d) removing any protecting groups from said intermediate compound.

27. The process as defined in claim 26, wherein said reacting of said cyclic sulfate and said heterocycle ring compound is performed in a polar solvent.

28. The process as defined in claim 27, wherein said polar solvent comprises hexafluorisopropanol.

29. The process as defined in claim 26, wherein said cyclic sulfate is derived from D-glucose.

30. The process as defined in claim 26, wherein said cyclic sulfate is a benzylidene-protected cyclic sulfate.

31. The process as defined in claim 30, wherein one or more benzylidene protecting groups are installed on said benzylidene-protected cyclic sulfate in the presence of the catalyst pyridinium—p-toluenesulfonate (PPTS).

32. The process as defined in claim 26, wherein said protected hydroxyl group of said heterocycle ring compound is p-methoxybenzyl.

33. The process as defined in claim 26, wherein said heterocycle ring compound is p-methoxybenzyl-protected 1,4-anhydro-4-seleno-D-arabinitol.

34. The process as defined in claim 26, wherein the removal of the protecting groups is performed by hydrogenolysis of said intermediate compound.

35. The process as defined in claim 26, wherein the removal of the protecting groups is performed by acid hydrolysis.

36. The process as defined in claim 35, wherein said acid hydrolyis is performed with trifluoroacetic acid.

37. The use of the compound (I) of claim 1 for inhibiting the activity of a glucosidase enzyme.

38. The use as defined in claim 37, wherein said glycosidase enzyme is selected from the group consisting of intestinal maltase-glucoamylase and pancreatic alpha amylase.

39. The use of Blintol for inhibiting the activity of intestinal maltase-glucoamylase.

40. A pharmaceutical composition comprising an effective amount of a compound according to claim 1 together with a pharmaceutically acceptable carrier.

42. A method of treating a carbohydrate metabolic disorder in an affected patient comprising the step of administering to said patient a therapeutically effective amount of a compound according to claim 1.

43. The method of claim 42, wherein said carbohydrate metabolic disorder is non-insulin dependent diabetes.

44. A pharmaceutical composition comprising an effective amount of a compound according to claim 8 together with a pharmaceutically acceptable carrier.

45. A method of treating a carbohydrate metabolic disorder in an affected patient comprising the step of administering to said patient a therapeutically effective amount of a compound according to claim 8.

46. The method of claim 45, wherein said carbohydrate metabolic disorder is non-insulin dependent diabetes.

47. Salacinol produced by a synthetic process defined in any one of schemes 7, 8, 9, 10, 10a, 10b and 10c.

48. Blintol produced by a synthetic process defined in any one of schemes 11, 12, 12a, 12b, 12c, 12d, 12e, and 12f.

49. A pharmaceutical comprising an effective amount of Salacinol and Blintol together with a pharmaceutically acceptable carrier.
